SEQUENCE LISTING (82 sequences)

SEQ ID NO: 1 - DNA, 950 bp, Camelina
  misc_feature (3)..(3): n is a, c, g, or t
tanagcggtggacttctagatctttctaaacctcttcagaccggagattcaccaccagcaccttcaaccgcagggtggaatcaatcttattgaccagcatcatcatcagcatcagcagcaacaacaacaacaacaacagcmaccgtcggatgattcaagagaatctgaacactcaaacaaggatcatcatcaacagggtcgaccccgattcagacccgaatacatcaagctcaacacccgggaaacgtccacgtggacgtccgccaggatctaagaacaaagcaaagccaccgatcatagtaacccgtgacagccccaacgcgcttagatctcacgtccttgaagtatctcccggagctgatatagttgagagtgtttccacttacgctaggcggagagggagaggcgtctccgttttaggagggaacggcaccgtttctaacgtcactctccgtcagccagtcactcccggaaacggtggtggtgtgtccggaggaggaggaggaggagttgtgactttacatggaagatttgagattctttcactaacggggactgttttgccacctcctgcgccgcctggtgcaggtggtttgtcaatatttctagccggtgggcaaggtcaggttgttggaggaagcgtggtggctccgcttattgcatcagctccagttatactaatggctgcttcgttctcaaatgcggttttcgagagactaccaatggaagaggaagaagaagaaggtgctggtgctggcggagggggaggaggaggaccaccgcagatgcagcaagctccctcagcatcgcctccgtcaggcgtgaccggtcagggacagttaggaggtaatgtgggtggttatgggttttctggtgatcctcatttgcttggatggggagctggaacaccttcaagaccactattttaatcgaanttaaantccngaatt

SEQ ID NO: 2 - DNA, 780 bp, Camelina
atgattcaagagaatctgaacactcaaacaaggatcatcatcaacagggtcgaccccgattcagacccgaatacatcaagctcaacacccgggaaacgtccacgtggacgtccgccaggatctaagaacaaagcaaagccaccgatcatagtaacccgtgacagccccaacgcgcttagatctcacgtccttgaagtatctcccggagctgatatagttgagagtgtttccacttacgctaggcggagagggagaggcgtctccgttttaggagggaacggcaccgtttctaacgtcactctccgtcagccagtcactcccggaaacggtggtggtgtgtccggaggaggaggaggaggagttgtgactttacatggaagatttgagattctttcactaacggggactgttttgccacctcctgcgccgcctggtgcaggtggtttgtcaatatttctagccggtgggcaaggtcaggttgttggaggaagcgtggtggctccgcttattgcatcagctccagttatactaatggctgcttcgttctcaaatgcggttttcgagagactaccaatggaagaggaagaagaagaaggtgctggtgctggcggagggggaggaggaggaccaccgcagatgcagcaagctccctcagcatcgcctccgtcaggcgtgaccggtcagggacagttaggaggtaatgtgggtggttatgggttttctggtgatcctcatttgcttggatggggagctggaacaccttcaagaccactattttaa

SEQ ID NO: 3 - protein, 259 aa, Camelina
MIQENLNTQTRIIINRVDPDSDPNTSSSTPGKRPRGRPPGSKNKAKPPIIVTRDSPNALRSHVLEVSPGADIVESVSTYARRRGRGVSVLGGNGTVSNVTLRQPVTPGNGGGVSGGGGGGVVTLHGRFEILSLTGTVLPPPAPPGAGGLSIFLAGGQGQVVGGSVVAPLIASAPVILMAASFSNAVFERLPMEEEEEEGAGAGGGGGGGPPQMQQAPSASPPSGVTGQGQLGGNVGGYGFSGDPHLLGWGAGTPSRPLF

SEQ ID NO: 4 - DNA, 873 bp, Camelina
  misc_feature (3)..(3): n is a, c, g, or t
cananngcggncangaangtggatactttcacaacctctttcagacctgaccttcatcgccaacttcaacytcagcctcatctccaccctctgcctcaacctcaacctcaacctgagcctcagcaacaacaatcagatgatgaatctgactccaacaaggatccgggttccgacccggttacctcgagttcaaactcctgggaagcgtccacgtgggcgtcctccgggatctaagaacaagccgaagccaccggtgatagtgacaagagatagccccaacgtgcttagatctcatgttcttgaaatctcatctggagccgacataattgagtgcgttaacacttacgctcgccggagagggaaaggtgtctccattctcagtggtaacggcacggtagctaacgtcagcatccgtcagccggcaacggctcatgcgactaatggtggagccggaggtgttgtttctttacatggaaggtttgaggtgctttccatcactggtacggtgttgccaccacctgcgcccccgggatccggtggtctttctgtctttcttgccggcacacaaggtcaggtggtcggaggactcgcggtgtctccgcttgtggcttcgggtccagtggtacttatggcttcatcgttctctaatgcaactttcgaacggcttccgcttgaggatgaaggaggagaaggcggaggaggagaagttggagagggaggtagtggagccggaggtggtggtccaccgcaggccacgtcggcatcttcaccaccgtctggagctggtcaaggacagttaagaggtaacatgagtggttatgatcagtttgccggtgatcctcatgtgcttggtgggagctcngccctccagcc

SEQ ID NO: 5 - DNA, 737 bp, Camelina
  misc_feature (726)..(726): n is a, c, g, or t
atgatgaatctgactccaacaaggatccgggttccgacccggttacctcgagttcaaactcctgggaagcgtccacgtgggcgtcctccgggatctaagaacaagccgaagccaccggtgatagtgacaagagatagccccaacgtgcttagatctcatgttcttgaaatctcatctggagccgacataattgagtgcgttaacacttacgctcgccggagagggaaaggtgtctccattctcagtggtaacggcacggtagctaacgtcagcatccgtcagccggcaacggctcatgcgactaatggtggagccggaggtgttgtttctttacatggaaggtttgaggtgctttccatcactggtacggtgttgccaccacctgcgcccccgggatccggtggtctttctgtctttcttgccggcacacaaggtcaggtggtcggaggactcgcggtgtctccgcttgtggcttcgggtccagtggtacttatggcttcatcgttctctaatgcaactttcgaacggcttccgcttgaggatgaaggaggagaaggcggaggaggagaagttggagagggaggtagtggagccggaggtggtggtccaccgcaggccacgtcggcatcttcaccaccgtctggagctggtcaaggacagttaagaggtaacatgagtggttatgatcagtttgccggtgatcctcatgtgcttggtgggagctcngccctccagcc

SEQ ID NO: 6 - protein, 245 aa, Camelina
MMNLTPTRIRVPTRLPRVQTPGKRPRGRPPGSKNKPKPPVIVTRDSPNVLRSHVLEISSGADIIECVNTYARRRGKGVSILSGNGTVANVSIRQPATAHATNGGAGGVVSLHGRFEVLSITGTVLPPPAPPGSGGLSVFLAGTQGQVVGGLAVSPLVASGPVVLMASSFSNATFERLPLEDEGGEGGGGEVGEGGSGAGGGGPPQATSASSPPSGAGQGQLRGNMSGYDQFAGDPHVLGGSSALQ

SEQ ID NO: 7 - DNA, 936 bp, Arabidopsis thaliana
atggaaggcggttacgagcaaggcggtggagcttctagatacttccataacctctttagaccggagattcaccaccaacagcttcaaccgcagggcgggatcaatcttatcgaccagcatcatcatcagcaccagcaacatcaacaacaacaacaaccgtcggatgattcaagagaatctgaccattcaaacaaagatcatcatcaacagggtcgacccgattcagacccgaatacatcaagctcagcaccgggaaaacgtccacgtggacgtccaccaggatctaagaacaaagccaagccaccgatcatagtaactcgtgatagccccaacgcgcttagatctcacgttcttgaagtatctcctggagctgacatagttgagagtgtttccacgtacgctaggaggagagggagaggcgtctccgttttaggaggaaacggcaccgtatctaacgtcactctccgtcagccagtcactcctggaaatggcggtggtgtgtccggaggaggaggagttgtgactttacatggaaggtttgagattctttcgctaacggggactgttttgccacctcctgcaccgcctggtgccggtggtttgtctatatttttagccggagggcaaggtcaggtggtcggaggaagcgttgtggctccccttattgcatcagctccggttatactaatggcggcttcgttctcaaatgcggttttcgagagactaccgattgaggaggaggaagaagaaggtggtggtggcggaggaggaggaggaggagggccaccgcagatgcaacaagctccatcagcatctccgccgtctggagtgaccggtcagggacagttaggaggtaatgtgggtggttatgggttttctggtgatcctcatttgcttggatggggagctggaacaccttcaagaccacctttttaa

SEQ ID NO: 8 - protein, 311 aa, Arabidopsis thaliana
MEGGYEQGGGASRYFHNLFRPEIHHQQLQPQGGINLIDQHHHQHQQHQQQQQPSDDSRESDHSNKDHHQQGRPDSDPNTSSSAPGKRPRGRPPGSKNKAKPPIIVTRDSPNALRSHVLEVSPGADIVESVSTYARRRGRGVSVLGGNGTVSNVTLRQPVTPGNGGGVSGGGGVVTLHGRFEILSLTGTVLPPPAPPGAGGLSIFLAGGQGQVVGGSVVAPLIASAPVILMAASFSNAVFERLPIEEEEEEGGGGGGGGGGGPPQMQQAPSASPPSGVTGQGQLGGNVGGYGFSGDPHLLGWGAGTPSRPPF

SEQ ID NO: 9 - DNA, 1036 bp, Arabidopsis thaliana
ctgccatggacggtggttacgatcaatccggaggagcttctagatactttcacaacctcttcaggcctgagcttcatcaccagcttcaacctcagcctcaacttcaccctttgcctcagcctcagcctcaacctcagcctcagcagcagaattcagatgatgaatctgactccaacaaggatccgggttccgacccagttacctctggttcaaccgggaaacgtccacgtggacgtcctccgggatccaagaacaagccgaagccaccggtgatagtgactagagatagccccaacgtgcttagatctcatgttcttgaagtctcatctggagccgacatagtcgagagcgttaccacttacgctcgcaggagaggaagaggagtctccattctcagtggtaacggcacggtggctaacgtcagtctccggcagccggcaacgacagcggctcatggggcaaatggtggaaccggaggtgttgtggctctacatggaaggtttgagatactttccctcacaggtacggtgttgccgccccctgcgccgccaggatccggtggtctttctatctttctttccggcgttcaaggtcaggtgattggaggaaacgtggtggctccgcttgtggcttcgggtccagtgatactaatggctgcatcgttctctaatgcaactttcgaaaggcttccccttgaagatgaaggaggagaaggtggagagggaggagaagttggagagggaggaggaggagaaggtggtccaccgccggccacgtcatcatcaccaccatctggagccggtcaaggacagttaagaggtaacatgagtggttatgatcagtttgccggtgatcctcatttgcttggttggggagccgcagccgcagccgcaccaccaagaccagccttttagaattgaaaattatgtccgtaacatagctgtaaccaaatttcatttctcaaaattaaaagaaaaaaaaaatcatcttcattgtttggggatcgtttggtttttaatttagttgatcatatatg

SEQ ID NO: 10 - protein, 302 aa, Arabidopsis thaliana
MDGGYDQSGGASRYFHNLFRPELHHQLQPQPQLHPLPQPQPQPQPQQQNSDDESDSNKDPGSDPVTSGSTGKRPRGRPPGSKNKPKPPVIVTRDSPNVLRSHVLEVSSGADIVESVTTYARRRGRGVSILSGNGTVANVSLRQPATTAAHGANGGTGGVVALHGRFEILSLTGTVLPPPAPPGSGGLSIFLSGVQGQVIGGNVVAPLVASGPVILMAASFSNATFERLPLEDEGGEGGEGGEVGEGGGGEGGPPPATSSSPPSGAGQGQLRGNMSGYDQFAGDPHLLGWGAAAAAAPPRPAF

SEQ ID NO: 11 - DNA, 1327 bp, Arabidopsis thaliana
tcaccgacccagctctttcacatatgtagacctctctctctctaattattcagtaaggttttgttctctcttgtcgtcttcttgttttgtccgtagcattattaataatcaaactcagtctctgaaatactctcagtgacaaaccacttctgtcgacttttgtctttgtctctcactagctataaccaaaaaaatccaaaaacccaaaccacagtttcttgtttttgttatcaatgtctagttatatgcaccctcttctagggcaagaactgcatctacagagacctgaagattccagaaccccacctgatcaaaataacatggaacttaacagatctgaagcagacgaagcaaaggccgagaccactcccaccggtggagccaccagctcagccacagcctctggctcttcctccggacgtcgtccacgtggtcgtcctgcaggttccaaaaacaaacccaaacctccgacgattataactagagatagtcctaacgtccttagatcacacgttcttgaagtcacctccggttcggacatatccgaggcagtctccacctacgccactcgtcgcggctgcggcgtttgcattataagcggcacgggtgcggtcactaacgtcacgatacggcaacctgcggctccggctggtggaggtgtgattaccctgcatggtcggtttgacattttgtctttgaccggtactgcgcttccaccgcctgcaccaccgggagcaggaggtttgacggtgtatctagccggaggtcaaggacaagttgtaggagggaatgtggctggttcgttaattgcttcgggaccggtagtgttgatggctgcttcttttgcaaacgcagtttatgataggttaccgattgaagaggaagaaaccccaccgccgagaaccaccggggtgcagcagcagcagccggaggcgtctcagtcgtcggaggttacggggagtggggcccaggcgtgtgagtcaaacctccaaggtggaaatggtggaggaggtgttgctttctacaatcttggaatgaatatgaacaattttcaattctccgggggagatatttacggtatgagcggcggtagcggaggaggtggtggcggtgcgactagacccgcgttttagagttttagcgttttggtgacaccttttgttgcgtttgcgtgtttgacctcaaactactaggctactagctatagcggttgcgaaatgcgaatattaggttttatttatttattttttgtttctcttaaaacttggtgtgtgaaaaaagaaaacgaacgcttaaatttcctttttctgtttgtttttatgaggcc

SEQ ID NO: 12 - protein, 299 aa, Arabidopsis thaliana
MSSYMHPLLGQELHLQRPEDSRTPPDQNNMELNRSEADEAKAETTPTGGATSSATASGSSSGRRPRGRPAGSKNKPKPPTIITRDSPNVLRSHVLEVTSGSDISEAVSTYATRRGCGVCIISGTGAVTNVTIRQPAAPAGGGVITLHGRFDILSLTGTALPPPAPPGAGGLTVYLAGGQGQVVGGNVAGSLIASGPVVLMAASFANAVYDRLPIEEEETPPPRTTGVQQQQPEASQSSEVTGSGAQACESNLQGGNGGGGVAFYNLGMNMNNFQFSGGDIYGMSGGSGGGGGGATRPAF

SEQ ID NO: 13 - DNA, 1566 bp, Arabidopsis thaliana
aacattttcttattttccttcctaatttccaacctctgttcttagcaatatattttttctccaaaaataattctcagtttgattttcttcttctagctcttaagtatatttctttgttgttatttatcttttaatcctttaatctcatctttgtttatctttaatcaaaacccaaaatttacatgggttcttgaaaatctagaagaaataaaggaaacataacaaaaatagaaagaaaaagaagctaatggtcttaaatatggagtctaccggagaagctgttagatcaaccaccggtaacgacggtggtattacggtggttagatccgacgcgccgtcagatttccacgtagctcaaagatcagaaagctcaaaccaatctcccacctctgtcactcctcctccaccacagccatcgtctcatcacacagctcctccgccgctgcaaatttcgacggtgacgactacgactacgacggccgcgatggaaggtatctccggtggactgatgaagaagaagcgtggacggccaaggaagtatggaccggacgggactgttgtagcgttatctcctaaaccgatttcatcagcgccggcgccgtcgcatcttccgccgccgagttcacacgtcatcgatttctccgcttctgagaaacgtagcaaagtgaaaccaacgaactcgtttaacagaacaaagtatcatcaccaagttgagaatttgggtgaatgggctccttgctccgtcggtggtaatttcacacctcatataatcacagtcaacaccggcgaggatgtaacaatgaagataatctcgttttcgcaacaaggacctcgctctatttgtgttctgtcagcaaacggtgttatttcaagcgttacacttcgtcagccagattcctctggcggcacattgacatacgaaggtcggtttgagatattatcattatccgggtcattcatgcctaatgattcaggcggaacacgaagtagaacgggaggaatgagtgtatcgttagcaagtcccgatggacgtgtagtaggcggtggcctcgccggtttactagtagccgcgagtccggttcaggtggttgtaggaagttttttagcgggcactgaccatcaagatcagaaaccgaaaaagaacaaacatgatttcatgttgtcgagtcctaccgctgcaattcctatctctagtgcagctgatcaccggacaatccattcggtctcgtctcttccggtcaataataatacatggcagacttctttagcttccgatccaagaaacaagcataccgatattaatgtcaatgtaacttgaaatccaatctttctctgtattttctgttaacaagtttgatttggttgtttatctacattaggattttactaaaatggtagtattatttatagggttttagggtctttattttggttccactgttgtcacttgtaggataatttctatctttttattaggattgaatcatcttgtttccttaattttttgctaacgagtcatggtcctggttttgcatgtcattgtagtttcatatccgtgtaaaagaa

SEQ ID NO: 14 - protein, 356 aa, Arabidopsis thaliana
MVLNMESTGEAVRSTTGNDGGITVVRSDAPSDFHVAQRSESSNQSPTSVTPPPPQPSSHHTAPPPLQISTVTTTTTTAAMEGISGGLMKKKRGRPRKYGPDGTVVALSPKPISSAPAPSHLPPPSSHVIDFSASEKRSKVKPTNSFNRTKYHHQVENLGEWAPCSVGGNFTPHIITVNTGEDVTMKIISFSQQGPRSICVLSANGVISSVTLRQPDSSGGTLTYEGRFEILSLSGSFMPNDSGGTRSRTGGMSVSLASPDGRVVGGGLAGLLVAASPVQVVVGSFLAGTDHQDQKPKKNKHDFMLSSPTAAIPISSAADHRTIHSVSSLPVNNNTWQTSLASDPRNKHTDINVNVT

SEQ ID NO: 15 - DNA, 1501 bp, Arabidopsis thaliana
cataaataaatctaattttcctcactttcctctcaaacatttcctaattccatcctaataattttcaaagctttaattctaagaaataatatctacaagaaaatattatctcatgtatttcttcttcctccttgatttttaatctgatttttaacctttggatttggaaaattatatcttcttctgtcccaaatctatttttaggtttgtcaaaacgattattttgtttaataatcgtaataggtatggagactaccggagaagttgttaaaacaaccaccgggagcgacggaggcgttacggtggtgagatccaacgcgccgtcagacttccacatggctccgaggtcagaaacttcaaacacacctcccaactccgtcgctcctcctcctcctccaccgccgcaaaactcctttactccgtcggcggctatggatggtttctcaagcggaccgataaagaagagacgtgggcgccctaggaagtacggacacgacggagcagcggtgacgctatctccgaatccgatatcatcagccgcaccaacgacttctcacgtcatcgatttctcgacgacatcggagaaacgtggcaaaatgaaaccagcaactccaactccaagctcattcatcaggccaaagtaccaggtcgagaatttaggtgaatggtctccttcctctgccgccgctaatttcacgccgcatattattacggtgaatgcaggcgaggacgttacgaagaggataatatcattttctcaacaagggtctctagctatttgcgttttatgcgcaaacggtgtcgtttcgagcgttacacttcgtcagcctgattcatctggtggtacattgacctatgagggtcggtttgagatattgtcactatctggaacattcatgcctagtgactcagacgggacacgaagcagaacaggcgggatgagcgtgtcgcttgctagccctgatggacgtgtagtaggtggtggtgttgctggcttgctggttgcagccactcctattcaagtggttgtaggaactttcttaggtggaacaaaccagcaagaacagacaccgaagccgcataaccacaacttcatgtcttctccattaatgccaacttcttcgaatgtagctgatcatcgaaccatccgtcccatgacatctagtctcccgatcagtacatggacaccgtcttttccttctgattcacgacacaagcattctcatgactttaatatcactttgacgtgatttcttccttgaagaactcgtagatcctctgtattttggtttccagtttagggctctacatgttagactctcaaagtctaggtgttatgttggtctgtcacttaggattgtcacttaggattgttagaccatctccatcaatggtttctcattgagaaactgttcaatataaaaataaaatataatcaaaaagtatgagagagagtataattagtctctcaagtgagaaactctaagacacctacaaaaac

SEQ ID NO: 16 - protein, 334 aa, Arabidopsis thaliana
METTGEVVKTTTGSDGGVTVVRSNAPSDFHMAPRSETSNTPPNSVAPPPPPPPQNSFTPSAAMDGFSSGPIKKRRGRPRKYGHDGAAVTLSPNPISSAAPTTSHVIDFSTTSEKRGKMKPATPTPSSFIRPKYQVENLGEWSPSSAAANFTPHIITVNAGEDVTKRIISFSQQGSLAICVLCANGVVSSVTLRQPDSSGGTLTYEGRFEILSLSGTFMPSDSDGTRSRTGGMSVSLASPDGRVVGGGVAGLLVAATPIQVVVGTFLGGTNQQEQTPKPHNHNFMSSPLMPTSSNVADHRTIRPMTSSLPISTWTPSFPSDSRHKHSHDFNITLT

SEQ ID NO: 17 - DNA, 1335 bp, Arabidopsis thaliana
atggaggagagagaaggaaccaacatcaacaacaacatcactagcagtttcggcttgaagcagcaacatgaagctgctgcttctgatggtggttactcaatggacccaccaccaagacccgaaaaccctaacccgtttttagtcccacccactactgtccccgcggccgccaccgtagcagcagctgttactgagaatgcggctactccgtttagcttaacaatgccgacggagaacacttcagctgagcagctgaaaaagaagagaggtaggccgagaaagtataatcccgatgggactcttgtcgtgactttatcgccgatgccaatctcgtcctctgttccgttgacgtcggagtttcctccaaggaaacgaggaagaggacgtggcaagtctaatcgatggctcaagaagtctcaaatgttccaattcgatagaagtcctgttgataccaatttggcaggtgtaggaactgctgattttgttggtgccaactttacacctcatgtactgatcgtcaacgccggagaggatgtgacgatgaagataatgacattctctcaacaaggatctcgtgctatctgcatcctttcagctaatggtcccatctccaatgttacgcttcgtcaatctatgacatccggtggtactctaacttatgagggtcgttttgagattctctctttgacgggttcgtttatgcaaaatgactctggaggaactcgaagtagagctggtggtatgagtgtttgccttgcaggaccagatggtcgtgtctttggtggaggactcgctggtctctttcttgctgctggtcctgtccaggtaatggtagggacttttatagctggtcaagagcagtcacagctggagctagcaaaagaaagacggctaagatttggggctcaaccatcttctatctcctttaacatatccgcagaagaacggaaggcgagattcgagaggcttaacaagtctgttgctattcctgcaccaaccacttcatacacgcatgtaaacacaacaaatgcggttcacagttactatacaaactcggttaaccatgtcaaggatcccttctcgtctatcccagtaggaggaggaggaggtggagaggtaggagaagaagagggtgaagaagatgatgatgaattagaaggtgaagacgaagaattcggaggcgatagccaatctgacaacgagattccgagctgatgatgatcatacggtttcttttcgcggatttgttaggtttgatggatttcagattttggttgattgtttttattaacacagaatgtttagaagctgctatctttaggttcccatcctctt

SEQ ID NO: 18 - protein, 404 aa, Arabidopsis thaliana
MEEREGTNINNNITSSFGLKQQHEAAASDGGYSMDPPPRPENPNPFLVPPTTVPAAATVAAAVTENAATPFSLTMPTENTSAEQLKKKRGRPRKYNPDGTLVVTLSPMPISSSVPLTSEFPPRKRGRGRGKSNRWLKKSQMFQFDRSPVDTNLAGVGTADFVGANFTPHVLIVNAGEDVTMKIMTFSQQGSRAICILSANGPISNVTLRQSMTSGGTLTYEGRFEILSLTGSFMQNDSGGTRSRAGGMSVCLAGPDGRVFGGGLAGLFLAAGPVQVMVGTFIAGQEQSQLELAKERRLRFGAQPSSISFNISAEERKARFERLNKSVAIPAPTTSYTHVNTTNAVHSYYTNSVNHVKDPFSSIPVGGGGGGEVGEEEGEEDDDELEGEDEEFGGDSQSDNEIPS

SEQ ID NO: 19 - DNA, 1740 bp, Arabidopsis thaliana
ctgaatcttacgtcctctggatctttcttctctctctctctctcttgtctatactttaagtgtctgtctgaagaagcttgaatctgtcgtgatcatcaagattcaagttctgtctttttgagtctttttcttctacttctattgagtttctcttccttaattgtcggaatcagaatcttttcagatggaggagagagaaggaactaacatcaacaacatcccaaccagttttggtctgaaacaacatgaaactcctcttcctcctcctggttacccaccacggtctgaaaaccctaatctttttccggtgggtcaatccagcacttcctccgccgccgccgcggtgaaaccttctgagaatgttgctcctccttttagcttaacaatgccggtggagaattcttcttctgagttgaagaagaagagagggagaccaagaaagtataaccctgacggctcactcgctgtgactctctctcctatgcctatctcatcctccgttccgttgacgtcggagtttggttctcggaaacgaggaagaggtcgaggaagaggcagaggaagaggacgaggacgtggacaaggacaaggaagcagagagcccaataacaacaacaacgacaacaattggctcaagaatcctcagatgttcgaatttaacaacaacactcctacttctggtggaggaggacctgctgaaattgtcagtccaagttttacacctcatgtgctcacagtaaatgccggtgaggatgtgacaatgaagataatgacattctctcaacaaggctcgcgtgctatttgtattctttcagcgaacggtcccatatccaatgttacacttcgtcaatctatgacatctggtggtactctcacttatgagggtcattttgagattctttctttgacgggttcgtttataccaagcgagagtggaggaacccgaagcagagctggtgggatgagtgtctctcttgcaggacaagatggtcgtgtctttggtggtggacttgctggtctctttattgccgctggtcctgttcaggtaatggtagggagttttatagcgggtcaggaggaatcgcagcagcagcagcagcagataaagaagcaaagaagggaaagactcgggatcccgacaacaacacaagcttctaatatctcattcggtggctcagcggaagatcctaaggctagatacgggctcaacaagcctgttgttattcagccaccaccggtgtctgcaccacctgtgtccttttcgcatgaaccaagtactaacaccgtccatggttactatgcaaataacacagctaaccatatcaaggatctcttctcttccctccctggagaagatagggaagaagatgaggatgatttagaaggtgaagatgatgaagaattcggaggccatagcgaatctgacaccgaggttccaagctgatgatcgatggaaagaatccgacatatatgtgttatgaatcttgagttgttttatttcggtgtcttcagatttttttagagcgtaatggtattttttttctttcagattgttagttgttaaagtcttaaacagagatatttcactaaaagttagggtttactagaggatgtaatctttagggttctttgacttgtgtctttcttttaatcctcagatggttgttgtaggcttgtagccaatcttagtgtgtgttcaaactctctccttcaatcaaactccccaatgacttcattttg

SEQ ID NO: 20 - protein, 419 aa, Arabidopsis thaliana
MEEREGTNINNIPTSFGLKQHETPLPPPGYPPRSENPNLFPVGQSSTSSAAAAVKPSENVAPPFSLTMPVENSSSELKKKRGRPRKYNPDGSLAVTLSPMPISSSVPLTSEFGSRKRGRGRGRGRGRGRGRGQGQGSREPNNNNNDNNWLKNPQMFEFNNNTPTSGGGGPAEIVSPSFTPHVLTVNAGEDVTMKIMTFSQQGSRAICILSANGPISNVTLRQSMTSGGTLTYEGHFEILSLTGSFIPSESGGTRSRAGGMSVSLAGQDGRVFGGGLAGLFIAAGPVQVMVGSFIAGQEESQQQQQQIKKQRRERLGIPTTTQASNISFGGSAEDPKARYGLNKPVVIQPPPVSAPPVSFSHEPSTNTVHGYYANNTANHIKDLFSSLPGEDREEDEDDLEGEDDEEFGGHSESDTEVPS

SEQ ID NO: 21 - DNA, 1806 bp, Arabidopsis thaliana
ctgagatttgctttctctaccagagagactgtaacagtagtagtagctacttttgcttgttgtcttcatttcaaatctgcaattttactttctttttgagttctaagtgaactcaacatttagcttctttttcttttctaggtttcataaaaacagtttggatcttaagctcacagctgaaaataacccgactttgattactgggtatataaaagccgccattgttgttcttttcactctttgggttattagggtttacgatttctgggtggtagataaatttgttcttcaaaaaagtttccttttttaaaaaatctcttagattcttaaagaattgtaatttagggttttaaatgcatggatgggagagaagctatggcatttccaggctcgcattctcagttctatcttcagagaggagtttttaccaatcttacaccttcccaggtcgcgagtggtcttcacgcgccgccgccacctccgggaatgaggccaatgtcaaaccctaacattcaccaccctcaggctagcaatccaggacctcctttctccatggctgagcacaggcattctgatttcggacacagcattcacatggggatggcttctcctgctgcggtgcagcctactctgcagctgccgcctccaccgtcggagcaaccgatggttaagaagaaacgtggaaggccaagaaagtatgttcctgatggacaagtttcattggggctttctccaatgccttgtgttagtaaaaagtctaaggactcttcttcaatgtctgatcctaatgcacctaaaagagccagaggtcgacctcccggaactggaaggaagcaacgcttggctaatcttggtgagtggatgaatacatcagctggacttgcttttgcacctcatgtgatcagtgttggatcaggagaagatattgtttcgaaagttttgtctttttcacaaaaaagacctcgggctctttgtataatgtcaggcactggaacggtttcttcagtcactctgcgtgaacccgcttcaacaacgccttccttaacatttgagggacgttttgagattctaagtttaggtggatcttatctggtgaatgaagaaggtggatccaaaagtcgaacaggcggtttgagtgtctctctttctggtcctgaaggtcatgttatcggcggtggaattggaatgcttatcgcagccagcctcgttcaggtggtggcttgtagtttcgtgtacggagcaagtgcaaagtctaataataacaataacaagaccatcaaacaagaaataaaacctaagcaagagccgaccaatagcgaaatggagaccacacctggtagtgcaccagaagctgcagcatcgacgggtcagcatacgccacagaacttcccagctcagggaatgagcggatggcccgtttcaggctcaggctcaggcagatcacttgactctagcagaaacccactcactgatattgacttgactcgcgggtgattcaaaacaaaaacattattagtctttcttcctatttactccgactactaccctttagtcttggaagcagcagcagcagcatacattatgtgagtgctgtacatatctttgttgttgtagatttctctctgggaatgttaaaaccagacatttcaggattgagaccagaactctcagttccttattgtaacattctcaatgtaacagaaccaccccactttcaggctactcaacgagattgtaactcttttatatcactcattaaagcttcctttggtttcttcaatatctgtgttcaattcggttttggtt

SEQ ID NO: 22 - protein, 378 aa, Arabidopsis thaliana
MDGREAMAFPGSHSQFYLQRGVFTNLTPSQVASGLHAPPPPPGMRPMSNPNIHHPQASNPGPPFSMAEHRHSDFGHSIHMGMASPAAVQPTLQLPPPPSEQPMVKKKRGRPRKYVPDGQVSLGLSPMPCVSKKSKDSSSMSDPNAPKRARGRPPGTGRKQRLANLGEWMNTSAGLAFAPHVISVGSGEDIVSKVLSFSQKRPRALCIMSGTGTVSSVTLREPASTTPSLTFEGRFEILSLGGSYLVNEEGGSKSRTGGLSVSLSGPEGHVIGGGIGMLIAASLVQVVACSFVYGASAKSNNNNNKTIKQEIKPKQEPTNSEMETTPGSAPEAAASTGQHTPQNFPAQGMSGWPVSGSGSGRSLDSSRNPLTDIDLTRG

SEQ ID NO: 23 - DNA, 1577 bp, Arabidopsis thaliana
attgaaagaagctctggcatttctctgctacagtgttggttttcagctgcagaattggtgaaaaaagtttggtgaatttcaggagcagagtatttaagtaattttggggaattagtgagaaaatggaggagaaaggtgaaataagtccaagtggggtcgtcacagttaagggagatgaagctttggttccaagaacagagtttcaacaaaaccctagttttctccaattcgtgagtcccacaacggtagtgactcctctccctcctccaccagctccgtcttcggctccggtaccaaccaccgtgactcctggctcagccacagcatcaaccggatcagatccgacgaaaaagaagagaggaagaccgaggaagtacgcaccagacggaagcctgaaccctaggtttttgcgaccaactctgtctccaactccaatctcatcttcgattccattgtctggagattatcaatggaaacgaggcaaagctcagcagcagcatcagcctcttgagttcgtcaagaaatctcacaaattcgagtatggaagcccagctcctactcctcctcttcctgggctctcatgctatgtgggtgcaaattttacgacccatcaatttactgtcaatggcggtgaggatgtgacaatgaaggttatgccgtattcgcaacaaggatctcgagctatctgcattctttctgcaactggttcgatctctaatgtcactcttggtcaacctaccaatgcaggtggcactcttacatatgagggtcggtttgagatactatcgctatctggttcctttatgcctactgaaaatggaggaacaaaaggtcgggctggtgggatgagcatttctttagccggaccaaatggcaatatatttggtggtggccttgccggtatgcttatagcagctggtcctgttcaggtggtaatgggaagtttcattgtgatgcatcaagcagaacaaaatcagaagaagaaacctagagttatggaggcttttgctccaccacaaccacaagcaccaccacaactgcaacaacaacaacctcctacttttaccataacaaccgtgaattcaacttctccttcggtgaacaccgtagaggagcagaaaccacaagcttatggtggtggtatagtgagaccgatggctcaaatgccttcttctttccaaaacgacaattcaactatgaacaatttcacaccggcctatcatggatacgggaatatgaacactggtactactcacaaagaagaacatgaagacgaagatggtggtgatgatgatgatgattcaggcgatactaggagccaatctcatagtggttgagcaattctctgatcaaagtcagaaagttttcttgtgtagaaaggaagggagcttaaaggagtgcttttttttagagggttgttttagttgtgtgagggttttcttcttttgtgtgcttttctaatttctagggttattcttctttgtgatcggtttagatttaatcatgtcgaagtcatagactaaacgatgttcttattgttgaggtcattgggattcaaatttagtaagacttttgtg

SEQ ID NO: 24 - protein, 404 aa, Arabidopsis thaliana
MEEKGEISPSGVVTVKGDEALVPRTEFQQNPSFLQFVSPTTVVTPLPPPPAPSSAPVPTTVTPGSATASTGSDPTKKKRGRPRKYAPDGSLNPRFLRPTLSPTPISSSIPLSGDYQWKRGKAQQQHQPLEFVKKSHKFEYGSPAPTPPLPGLSCYVGANFTTHQFTVNGGEDVTMKVMPYSQQGSRAICILSATGSISNVTLGQPTNAGGTLTYEGRFEILSLSGSFMPTENGGTKGRAGGMSISLAGPNGNIFGGGLAGMLIAAGPVQVVMGSFIVMHQAEQNQKKKPRVMEAFAPPQPQAPPQLQQQQPPTFTITTVNSTSPSVNTVEEQKPQAYGGGIVRPMAQMPSSFQNDNSTMNNFTPAYHGYGNMNTGTTHKEEHEDEDGGDDDDDSGDTRSQSHSG

SEQ ID NO: 25 - DNA, 1413 bp, Arabidopsis thaliana
gttttctttctactctgttttcaactggggacactatgtttttttttcttcttcttgtttggatcatataaatctgattttttcccggaaaataccgttgagaacttctccggcgtgagctgaccaggagaaatctgttcttgtgtttagttcaattctcggctgaggaacgttgactgttgactgatagagcaacttcagtgtgttgagaccaaacataaagcttcagttaaggataggatttgagaatggaaacaagcgacagaattagccctggtggtggtataggagcagaagttccatcagcgtatcatatggctccgagaccttcagatagccctgctaaccagtttatggggttgtctctccctcccatggaagccccaatgccttcttcaggggaagcctcagggaagaagaggagaggcagacctcgtaaatatgaagctaatggtgctcctttgccttcttcatcagttcctcttgtgaagaaacgagtaagaggcaagctcaatggttttgatatgaagaagatgcataagaccattgggttccattcttctggtgaaaggtttggtgttggtggtggtgttggtggtggtgttggatcaaatttcacgccgcatgtaatcacagtgaacactggtgaggatattactatgagaatcatctccttttcacaacaaggaccacgagctatttgtatcttatcagcaaatggagtgatatcaaacgttactcttcgccaacccgattcttgcggtggtactctcacctacgagggtcggtttgagatactgtctctgtctggttcattcatggaaacagagaatcaaggttcaaaaggtcggtctggtggcatgagtgtatctttggccggtcctgatggtcgtgttgtcggtggcggtgtcgctggcctgctcattgccgccactcctattcaggttgttgttggcagttttataacgagtgatcaacaagatcatcagaaaccgaggaagcaaagagtcgagcatgcccctgcagcagtcatgtcagtgcctcctcctccttctcctcctcctccggcggcttctgttttctcaccgacaaacccggacagagagcagccgccatcttcattcggtatctccagctggaccaacggacaggacatgccgagaaactcagccactgacatcaacatatctttaccagtcgactgagtttgagtttccagatggaagattaggtttcagattaactctctactgtctgtacaatggttaggaggattgttgaagctgaatgttactgtacgcttttctttattttagaacacaacttgagcttcttctacttttagggtaactttctctgatgacttgcctcttgttacttactatattgctagtactgaggtttaatcctttt

SEQ ID NO: 26 - protein, 318 aa, Arabidopsis thaliana
METSDRISPGGGIGAEVPSAYHMAPRPSDSPANQFMGLSLPPMEAPMPSSGEASGKKRRGRPRKYEANGAPLPSSSVPLVKKRVRGKLNGFDMKKMHKTIGFHSSGERFGVGGGVGGGVGSNFTPHVITVNTGEDITMRIISFSQQGPRAICILSANGVISNVTLRQPDSCGGTLTYEGRFEILSLSGSFMETENQGSKGRSGGMSVSLAGPDGRVVGGGVAGLLIAATPIQVVVGSFITSDQQDHQKPRKQRVEHAPAAVMSVPPPPSPPPPAASVFSPTNPDREQPPSSFGISSWTNGQDMPRNSATDINISLPVD

SEQ ID NO: 27 - DNA, 1161 bp, Arabidopsis thaliana
atggattccagagacatccccccgtcacataaccagcttcaaccaccaccgggaatgttaatgtctcattaccgtaaccctaacgccgccgcttcaccattaatggttcccacttccacatctcaaccgattcaacaccctcgtcttccttttggcaatcaacaacaatctcaaacgtttcatcagcagcaacaacaacaaatggatcagaagactcttgaatctcttggatttggtgatggatcaccttcttctcaaccgatgcgattcgggatcgatgatcagaatcagcaactgcaagtgaagaagaagcgaggaaggccgagaaagtatactcctgatggtagcattgctttaggtttagctcctacgtctcctcttctctctgcagcttctaattcttacggtgagggtggtgttggagatagtggtggaaatggaaactctgttgatccacctgttaaacgtaacagaggaaggcctcctggttctagtaagaaacagcttgatgctttaggaggaacttcaggagttgggtttacacctcatgtcattgaagtgaacacaggagaggacatagcgtcaaaggtgatggctttttcggatcaagggtcaagaacaatttgtattctctctgcaagtggtgcagtttctagagtgatgcttcgtcaagcttctcattctagtggaatcgttacttatgagggacgatttgagatcattactctctcaggctcagtcttgaattatgaggtaaatggttccaccaacagaagtggtaacttgagtgtggctttggctggacctgatggcggcatcgtaggtggcagtgtagttggtaatctagtagctgcaacacaagtccaggtgatagtgggaagctttgttgcagaagcaaagaaaccgaaacaaagtagtgttaacattgctcgggggcagaatcctgaaccggcttcagcgccggctaacatgttgaactttggatcagtctctcaaggaccatcgagcgagtcatcagaagagaatgagagcggttctcctgcaatgcaccgtgacaataataatgggatatatggagctcaacaacaacaacaacaacaacctcttcatcctcatcagatgcaaatgtaccaacatctttggtctaatcatggtcaataa

SEQ ID NO: 28 - protein, 386 aa, Arabidopsis thaliana
MDSRDIPPSHNQLQPPPGMLMSHYRNPNAAASPLMVPTSTSQPIQHPRLPFGNQQQSQTFHQQQQQQMDQKTLESLGFGDGSPSSQPMRFGIDDQNQQLQVKKKRGRPRKYTPDGSIALGLAPTSPLLSAASNSYGEGGVGDSGGNGNSVDPPVKRNRGRPPGSSKKQLDALGGTSGVGFTPHVIEVNTGEDIASKVMAFSDQGSRTICILSASGAVSRVMLRQASHSSGIVTYEGRFEIITLSGSVLNYEVNGSTNRSGNLSVALAGPDGGIVGGSVVGNLVAATQVQVIVGSFVAEAKKPKQSSVNIARGQNPEPASAPANMLNFGSVSQGPSSESSEENESGSPAMHRDNNNGIYGAQQQQQQQPLHPHQMQMYQHLWSNHGQ

SEQ ID NO: 29 - DNA, 1375 bp, Arabidopsis thaliana
atggatcgaagagatgcaatgggattatccgggtcaggttcttactatatccatagaggattatccgggtcgggtcctccaacgtttcatggatcaccacagcaacagcaaggtcttcgtcacttacctaatcaaaactctccattcgggtcaggctccactggtttcggatctccttctttacacggtgatccttctctggcaacagcagccggaggagccggagctcttcctcatcatatcggcgttaatatgattgctcctcctccacctcccagtgaaactccgatgaaacgaaagagaggacggcctagaaaatacggtcaagacggctctgtttctttggctctgtcgtcttcctctgtttcgaccattactcccaacaactctaacaaacgcggccgtggtcgacctccgggctccggcaagaaacagagaatggcttccgttggtgaactgatgccttcatcttctggaatgagcttcacgccacatgttatcgcggtttcaataggagaagatattgcatcaaaggttatagctttctctcaacaaggtccgagagccatttgcgttttatctgcaagtggtgcagtctctactgcaacacttattcaaccatcagcatctcccggagccattaaatacgagggccggtttgaaatcctagcgttatcaacatcttatatagtggcaactgatggaagcttccgtaaccgaactggaaacttatcggtttcgcttgctagccccgatgggcgtgtgattggcggtgccattggtgggcctttaatagctgcaagtcctgttcaggttattgtagggagctttatatgggcagctccaaagatcaagagcaagaaacgagaagaagaagcttctgaagttgttcaagaaactgatgatcaccacgttctggacaataataacaacacgatttcgcctgtccctcagcagcagccaaaccaaaacctgatttggtcaacaggttcaaggcaaatggatatgcgtcatgctcatgctgatattgatttaatgcgcggttgatgatagcgagaaagaactctgtgtatataaagcatggaatctaggaagaagaagaaggaatataagctaacctctgaacaaaagtatgtggaaatgttagggaaaaagattatgtggaaatgttagggaaaaagattaactctattagtgtacctctcatatctctaagcttgtttggttttactgtttctgtgactctgaagatttgcagagttcctttctttctctgttttagattgttcagtctttatgtaatttgcttgcaattctgattctacagcttagattcagtacattgtgtagaagtttacatgggaacctgaaaatt

SEQ ID NO: 30 - protein, 348 aa, Arabidopsis thaliana
MDRRDAMGLSGSGSYYIHRGLSGSGPPTFHGSPQQQQGLRHLPNQNSPFGSGSTGFGSPSLHGDPSLATAAGGAGALPHHIGVNMIAPPPPPSETPMKRKRGRPRKYGQDGSVSLALSSSSVSTITPNNSNKRGRGRPPGSGKKQRMASVGELMPSSSGMSFTPHVIAVSIGEDIASKVIAFSQQGPRAICVLSASGAVSTATLIQPSASPGAIKYEGRFEILALSTSYIVATDGSFRNRTGNLSVSLASPDGRVIGGAIGGPLIAASPVQVIVGSFIWAAPKIKSKKREEEASEVVQETDDHHVLDNNNNTISPVPQQQPNQNLIWSTGSRQMDMRHAHADIDLMRG

SEQ ID NO: 31 - DNA, 1389 bp, Arabidopsis thaliana
ctgaagtttcgcctcagtcatctctgagaagaaaatgtcaggatctgagacgggtttaatggcggcgaccagagaatcaatgcaatttacaatggctctccaccagcagcagcaacacagtcaagctcaacctcagcagtctcagaacaggccattgtcattcggtggagacgacggaactgctctttacaagcagccgatgagatcagtatcaccaccgcagcagtaccaacccaactcagctggtgagaattctgtcttgaacatgaacttgcccggaggtgagtctggaggcatgactggaactggaagtgagccagtgaaaaagaggagaggtagaccgaggaaatatgggcctgatagtggtgaaatgtcacttggtttgaatcctggagctccttctttcactgtcagccaacctagtagcggcggcgatggaggagagaagaagagaggaagacctcctggttcttctagcaaaaggctcaagcttcaagctttaggctcgactggaatcggatttacgcctcatgtacttaccgtgctggctggagaggatgtatcatccaagataatggcgttaactcataatggaccccgtgctgtgtgtgtcttgtctgcaaatggagccatctccaatgtgactctccgccagtctgccacatccggtggaactgttacatatgaggggagatttgagattctgtctttatcgggatctttccatttgctggagaacaatggtcaaagaagcaggacgggaggtctaagcgtgtcattatcaagtccggatggtaatgtcctcggtggcagtgtagctggtcttcttatagcagcatcacctgttcagattgttgttgggagtttcttaccagacggagaaaaagaaccaaaacagcatgtgggacaaatgggactgtcgtcacccgtattaccgcgtgtggccccaacgcaggtgctgatgactccaagtagcccacaatctcgaggcacaatgagtgagtcatcttgtggaggaggacatggaagccctattcatcagagcactggaggaccttacaataacaccattaacatgccctggaagtagccaagtgatctgtgtcggcttaaaaccaacaacttcccgttattagagtgatttatttctacatttggtttagactttctagttctgatggttatttctacagttggtttagactttctagttctgttcagacaaaaggagtttgataaattgaccgacctattttgtgtgtttgaggtactttcagaaccataggtgttcagaaattagaatgttctgtttaaggtagatcttttattttatgttgaggtactttcggaaccataagttgttcagaaattggaatatcctgattaatg

SEQ ID NO: 32 - protein, 351 aa, Arabidopsis thaliana
MSGSETGLMAATRESMQFTMALHQQQQHSQAQPQQSQNRPLSFGGDDGTALYKQPMRSVSPPQQYQPNSAGENSVLNMNLPGGESGGMTGTGSEPVKKRRGRPRKYGPDSGEMSLGLNPGAPSFTVSQPSSGGDGGEKKRGRPPGSSSKRLKLQALGSTGIGFTPHVLTVLAGEDVSSKIMALTHNGPRAVCVLSANGAISNVTLRQSATSGGTVTYEGRFEILSLSGSFHLLENNGQRSRTGGLSVSLSSPDGNVLGGSVAGLLIAASPVQIVVGSFLPDGEKEPKQHVGQMGLSSPVLPRVAPTQVLMTPSSPQSRGTMSESSCGGGHGSPIHQSTGGPYNNTINMPWK

SEQ ID NO: 33 - DNA, 1065 bp, Arabidopsis thaliana
atggatcgaagagacgcaatggcgttatccgggtcgggttcttactatatccaaagaggaatccccggttctggtcctcctcctcctcaaactcaaccaacgtttcacggatcacaaggatttcatcatttcaccaattccatctctccttttgggtcaaacccaaacccaaatccaaaccctggaggtgtctctactggattcgtgtctcctcctttacccgttgactcttctccggctgattcgtcagcggcggcggcgggagctttggttgctcctccttcaggtgacacgtctgtgaagcggaagagaggacggcctagaaaatatggacaagatggtggttctgtttcgttggcattgtctccttctatctccaacgtttccccgaactctaacaaacgtggccgtggaagacctcctggctccggcaagaagcaacggctatcttccattggtgaaatgatgccttcatcaactgggatgagcttcacaccgcatgtaatcgtagtttccattggtgaagacattgcttcaaaggttatatcgttctcgcatcaaggtccacgagcgatatgtgtcttatccgcaagtggtgctgtctctactgcaactcttcttcagccagcaccttctcatggaactattatatacgagggtctattcgagctcatatctctctcaacttcttatctgaacacaactgacaatgactacccaaaccgcactggaagtctagcggtctcacttgctagccccgatggtcgtgtcattggtggtggaattggaggtcctctaatagcagcaagccaagtccaggtcattgttggcagcttcatttgggcaattccgaaagggaagattaaaaaacgtgaagaaacttctgaagatgtccaagatactgatgctttggaaaacaacaacgataacacagcagcaacgtcacctcctgttcctcagcaaagtcagaacattgttcagactcctgtaggcatttggtcaactggttcaaggtcaatggatatgcatcacccccatatggacattgatctaatgcgtggatga

SEQ ID NO: 34 - protein, 354 aa, Arabidopsis thaliana
MDRRDAMALSGSGSYYIQRGIPGSGPPPPQTQPTFHGSQGFHHFTNSISPFGSNPNPNPNPGGVSTGFVSPPLPVDSSPADSSAAAAGALVAPPSGDTSVKRKRGRPRKYGQDGGSVSLALSPSISNVSPNSNKRGRGRPPGSGKKQRLSSIGEMMPSSTGMSFTPHVIVVSIGEDIASKVISFSHQGPRAICVLSASGAVSTATLLQPAPSHGTIIYEGLFELISLSTSYLNTTDNDYPNRTGSLAVSLASPDGRVIGGGIGGPLIAASQVQVIVGSFIWAIPKGKIKKREETSEDVQDTDALENNNDNTAATSPPVPQQSQNIVQTPVGIWSTGSRSMDMHHPHMDIDLMRG

SEQ ID NO: 35 - DNA, 1117 bp, Arabidopsis thaliana
atggacggaagagaagcaatggcatttccaggctcgcattctcagtactatcttcaaagaggagcctttactaatctcgcaccttcccaagtcgcgagtgggcttcacgcgccgccgccacatacgggattgaggccaatgtctaaccctaacattcatcaccctcaggctaacaatccaggacctcctttctcggattttggacacaccattcacatgggagtggtctcctctgcttctgatgctgatgtgcaaccgccaccgccaccgccaccaccagaggaaccgatggttaagaggaaacgtggacggccaagaaagtatggagaaccgatggttagtaataagtctagggactcttctccaatgtctgatcctaatgaacctaaacgggccagaggtcgacctcctggaactggaaggaagcaacgcttggctaatcttggtgagtggatgaatacttcagctggacttgcttttgcacctcatgtgatcagcattggagcaggagaagacattgctgcgaaagttttgtcattttcacaacaaagacctcgggctctttgtataatgtcaggcactggaaccatttcttcagtcactctgtgcaaacccggttcaaccgatcgtcacttaacatacgagggaccttttgagattataagttttggtggatcttatttggtgaatgaagaaggtggatccagaagtcgaacaggcggattgagtgtctctctttctcgtcccgatggtagtattattgccggtggagttgacatgcttatcgcagccaaccttgttcaggtggtggcatgtagttttgtatacggagcaagggcaaagactcataataacaataacaagaccatcagacaagaaaaggaaccaaatgaagaggacaacaatagtgaaatggagaccacaccgggtagtgcagctgaaccagcagcatctgcgggtcagcagacgccacagaacttctcttctcagggaataagggggtggcccggttcaggctcaggctctggcagatcacttgacatttgcagaaacccactcactgattttgatttgactcgtggatgatatacactattagtctttgaagcagcagcat

SEQ ID NO: 36 - protein, 361 aa, Arabidopsis thaliana
MDGREAMAFPGSHSQYYLQRGAFTNLAPSQVASGLHAPPPHTGLRPMSNPNIHHPQANNPGPPFSDFGHTIHMGVVSSASDADVQPPPPPPPPEEPMVKRKRGRPRKYGEPMVSNKSRDSSPMSDPNEPKRARGRPPGTGRKQRLANLGEWMNTSAGLAFAPHVISIGAGEDIAAKVLSFSQQRPRALCIMSGTGTISSVTLCKPGSTDRHLTYEGPFEIISFGGSYLVNEEGGSRSRTGGLSVSLSRPDGSIIAGGVDMLIAANLVQVVACSFVYGARAKTHNNNNKTIRQEKEPNEEDNNSEMETTPGSAAEPAASAGQQTPQNFSSQGIRGWPGSGSGSGRSLDICRNPLTDFDLTRG

SEQ ID NO: 37 - DNA, 1373 bp, Arabidopsis thaliana
atggattccagagagatccaccaccaacaacagcaacaacaacaacaacaacagcagcagcagcaacaacagcaacatctacaacaacagcaacaaccaccgccagggatgttaatgagtcaccacaattcctacaatcgaaaccctaacgccgccgccgctgttttaatgggtcacaacacctccacatctcaagctatgcatcaaagattaccttttggtggttctatgtcaccgcatcagcctcaacaacatcagtatcatcatcctcagcctcagcaacagatagatcagaagactcttgaatctcttggatttgatggatcgccttcttctgttgccgccactcaacaacattcgatgagatttgggatcgaccatcaacaggttaagaagaaacgaggtagacctaggaagtatgctgctgatggtggtggtggtggtggtggtggtagtaacattgctcttggtttggctcctacttcgcctcttccttctgcttctaattcttacggtggtggaaatgaaggaggtggtggtggtgatagcgccggagctaatgctaactcttccgatccacctgctaaacggaacagaggacgtcctcctggctccggtaagaagcagctcgatgctttaggaggaacaggaggagttgggttcacgcctcatgtcattgaggttaaaacaggagaggacatagctacgaagatattggcgtttacgaaccaagggccacgcgcaatctgtattctctcagctacaggagctgtaactaatgtgatgcttcgtcaagctaacaatagcaatcctactggaactgttaagtatgagggccgatttgaaatcatttctctgtcaggttctttcttgaattctgagagtaatggtactgtgaccaaaactggtaacttgagtgtgtcgctggctggacacgaaggccggattgtgggtggatgtgttgatggaatgctagtagctggatcacaagtccaggtcattgtgggaagctttgtaccagatggaaggaagcagaaacaaagtgcggggcgtgctcagaatactccggagccagcttcagcaccagccaatatgttgagctttggtggtgttggtggaccgggaagccctcgatctcaaggacaacaacactcgagcgagtcatcagaggaaaacgaaagtaattctccgttgcaccgtagaagcaacaacaacaacagcaacaatcatgggatatttggaaactctacacctcaaccgcttcaccaaattcctatgcagatgtaccagaatctctggcctggcaacagtcctcaataaacagatggttcatgggtcaagatttgaccgggtttgcttctctgttccttttg

SEQ ID NO: 38 - protein, 439 aa, Arabidopsis thaliana
MDSREIHHQQQQQQQQQQQQQQQQQHLQQQQQPPPGMLMSHHNSYNRNPNAAAAVLMGHNTSTSQAMHQRLPFGGSMSPHQPQQHQYHHPQPQQQIDQKTLESLGFDGSPSSVAATQQHSMRFGIDHQQVKKKRGRPRKYAADGGGGGGGGSNIALGLAPTSPLPSASNSYGGGNEGGGGGDSAGANANSSDPPAKRNRGRPPGSGKKQLDALGGTGGVGFTPHVIEVKTGEDIATKILAFTNQGPRAICILSATGAVTNVMLRQANNSNPTGTVKYEGRFEIISLSGSFLNSESNGTVTKTGNLSVSLAGHEGRIVGGCVDGMLVAGSQVQVIVGSFVPDGRKQKQSAGRAQNTPEPASAPANMLSFGGVGGPGSPRSQGQQHSSESSEENESNSPLHRRSNNNNSNNHGIFGNSTPQPLHQIPMQMYQNLWPGNSPQ

SEQ ID NO: 39 - DNA, 1327 bp, Arabidopsis thaliana
gaaattcttatttttgtgcgtatctctctaaaaaggaatggatcctaacgaaagccaccatcaccaccaacaacaacagctccatcacctccaccaacagcaacagcaacagcagcagcagcaacgactcacttctccttacttccaccaccaactacagcaccatcaccaccttccaaccaccgtagcaaccaccgcttctaccggaaacgccgttccatcttccaacaatgggcttttccctccgcagcctcagccacagcaccagcctaatgatgggtcatcttctctcgcggtgtaccctcattcagttccgtcctcggctgtgacggcgccgatggagccggtaaagaggaagaggggtcgaccaagaaagtatgtgacgccggaacaagccctagcggctaagaaattggcgtcttctgcgagtagttcgtctgctaaacagaggcgagagcttgctgctgttaccggtggtacggtatcgactaattccgggtcatccaagaaatctcagcttggttctgtcgggaaaactggacaatgttttactccgcatattgttaatatagctcctggcgaggatgtggtccagaaaattatgatgttcgcaaaccaaagcaagcatgaactatgcgttctttctgcatcaggcactatctctaatgcatccttgcgccaaccggctccatcaggaggcaacttaccatatgagggtcaatacgagattctctcactatctggatcctatatccgaactgaacaaggtggtaaatccggcggccttagcgtttctttatctgcttcagatggtcagatcatcggtggagcgattggtagccatctcacagctgctggcccggttcaggtgattcttggtacgtttcagcttgatagaaagaaggatgccgccgggagtggtgggaaaggggatgcttcaaacagtggaaaagtgtgtagagtttagatcccaagtagagaaacagaaggcgagcaaagaatctgaactgagagaggacttattagacagagactcgtctgaagggtctttaatcatagaaagaagttgctgagtgattgcttttgttcttcttcttggtacggtgtattatattaactccacaaccttttttttatactttcagtaacgattctccttcactttcaatttcattccttttttttatactctttttcttttcttataatattttttttggtttttctttcgtttgttactaaaaaaggaaatgctctttttgtgaaatatatacacttcgtttgttcggtttctacttatgttaattattcataacctatcttaatt

SEQ ID NO: 40 - protein, 309 aa, Arabidopsis thaliana
MDPNESHHHHQQQQLHHLHQQQQQQQQQQRLTSPYFHHQLQHHHHLPTTVATTASTGNAVPSSNNGLFPPQPQPQHQPNDGSSSLAVYPHSVPSSAVTAPMEPVKRKRGRPRKYVTPEQALAAKKLASSASSSSAKQRRELAAVTGGTVSTNSGSSKKSQLGSVGKTGQCFTPHIVNIAPGEDVVQKIMMFANQSKHELCVLSASGTISNASLRQPAPSGGNLPYEGQYEILSLSGSYIRTEQGGKSGGLSVSLSASDGQIIGGAIGSHLTAAGPVQVILGTFQLDRKKDAAGSGGKGDASNSGKVCRV

SEQ ID NO: 41 - DNA, 933 bp, Arabidopsis thaliana
atggcgaatccttggtgggtagggaatgttgcgatcggtggagttgagagtccagtgacgtcatcagctccttctttgcaccacagaaacagtaacaacaacaacccaccgactatgactcgttcggatccaagattggaccatgacttcaccaccaacaacagtggaagccctaatacccagactcagagccaagaagaacagaacagcagagacgagcaaccagctgttgaacccggatccggatccgggtctacgggtcgtcgtcctagaggtagacctcctggttccaagaacaaaccaaagagtccagttgttgttaccaaagaaagccctaactctctccagagccatgttcttgagattgctacgggagctgacgtggcggaaagcttaaacgcctttgctcgtagacgcggccggggcgtttcggtgctgagcggtagtggtttggttactaatgttactctgcgtcagcctgctgcatccggtggagttgttagtttacgtggtcagtttgagatcttgtctatgtgtggggcttttcttcctacgtctggctctcccgctgcagccgctggtttaaccatttacttagctggagctcaaggtcaagttgtgggaggtggagttgctggcccgcttattgcctctggacccgttattgtgatagctgctacgttttgcaatgccacttatgagaggttaccgattgaggaagaacaacagcaagagcagccgcttcaactagaagatgggaagaagcagaaagaagagaatgatgataacgagagtgggaataacggaaacgaaggatcgatgcagccgccgatgtataatatgcctcctaattttatcccaaatggtcatcaaatggctcaacacgacgtgtattggggtggtcctccgcctcgtgctcctccttcgtattga

SEQ ID NO: 42 - protein, 310 aa, Arabidopsis thaliana
MANPWWVGNVAIGGVESPVTSSAPSLHHRNSNNNNPPTMTRSDPRLDHDFTTNNSGSPNTQTQSQEEQNSRDEQPAVEPGSGSGSTGRRPRGRPPGSKNKPKSPVVVTKESPNSLQSHVLEIATGADVAESLNAFARRRGRGVSVLSGSGLVTNVTLRQPAASGGVVSLRGQFEILSMCGAFLPTSGSPAAAAGLTIYLAGAQGQVVGGGVAGPLIASGPVIVIAATFCNATYERLPIEEEQQQEQPLQLEDGKKQKEENDDNESGNNGNEGSMQPPMYNMPPNFIPNGHQMAQHDVYWGGPPPRAPPSY

SEQ ID NO: 43 - DNA, 774 bp, Arabidopsis thaliana
atggctggaggtacagctctaactccaacctctgtaggatccaagtctgttccaatgaggaaccatgaagcaacagagagaggcaacaccaacaacaacctgagagcattacccaaagccgtccaaccggtttcatcaatcgaaggagagatggctaagaggccacgtggcagacccgctggctccaagaacaaacccaaaccaccaatcattgtgactcacgacagtccaaattccctcagagctaacgccgttgagatcagctcaggttgtgacatctgtgagactttatcggattttgcaagaaggaaacagagaggtctctgcattctcagtgccaatggttgtgtcaccaatgtgacattaaggcaaccagcttcatcaggagcaattgtcacattacacggacgttacgagatcctctcattgcttggatcaatcttgcctccaccagcaccacttggaataactggtctgaccatttacttagccggacctcaaggacaggttgttggtggaggagtggttggtgggctaatcgcatctggtcctgttgttctcatggctgcatctttcatgaatgctgtttttgatcgtcttcctatggatgatgatgaagctgcctctatgcagaaccagcagtactaccagaatggaagatcccgtcctttagatgacattcatggactgcctcaaaatctgctcactaatggaaactcggcttctgatatctactcttgggggcctgcgcagcgtgtcatgtcgaaaccttaa

SEQ ID NO: 44 - protein, 257 aa, Arabidopsis thaliana
MAGGTALTPTSVGSKSVPMRNHEATERGNTNNNLRALPKAVQPVSSIEGEMAKRPRGRPAGSKNKPKPPIIVTHDSPNSLRANAVEISSGCDICETLSDFARRKQRGLCILSANGCVTNVTLRQPASSGAIVTLHGRYEILSLLGSILPPPAPLGITGLTIYLAGPQGQVVGGGVVGGLIASGPVVLMAASFMNAVFDRLPMDDDEAASMQNQQYYQNGRSRPLDDIHGLPQNLLTNGNSASDIYSWGPAQRVMSKP

SEQ ID NO: 45 - DNA, 831 bp, Arabidopsis thaliana
atgaaaggtgaatacagagagcaaaagagtaacgaaatgttttccaagcttcctcatcatcaacaacaacagcaacaacaacaacaacaacactctcttacctctcacttccacctctcctccaccgtaacccccaccgtcgatgactcctccatcgaagtggtccgacgtccacgtggcagaccaccaggttccaaaaacaaacctaaaccacccgtcttcgtcacacgtgacaccgaccctcctatgagtccttacatcctcgaagttccttcaggaaacgacgtcgtcgaagccatcaaccgtttctgccgccgtaaatccatcggagtctgcgtccttagtggctctggctctgtagctaacgtcactttacgtcagccatcaccggcagctcttggctctaccataactttccatggaaagtttgatctcctctccgtctccgcaacgtttctccctcctccgcctcgtacttccttgtctcctcccgtttctaacttcttcaccgtctctctcgctggacctcaaggacaaatcatcggagggttcgtcgctggtccacttatttcggcaggaacagtttacgtcatcgccgcaagtttcaacaacccttcttatcaccggttaccggcggaagaagagcaaaaacactcggcggggacaggggaaagagagggacaatctccgccggtctctggtggcggtgaagagtcaggacagatggcgggaagtggaggagagtcgtgtggggtatcaatgtacagttgccacatgggtggctctgatgttatttgggcccctacagccagagctccaccgccatactaa

SEQ ID NO: 46 - protein, 276 aa, Arabidopsis thaliana
MKGEYREQKSNEMFSKLPHHQQQQQQQQQQHSLTSHFHLSSTVTPTVDDSSIEVVRRPRGRPPGSKNKPKPPVFVTRDTDPPMSPYILEVPSGNDVVEAINRFCRRKSIGVCVLSGSGSVANVTLRQPSPAALGSTITFHGKFDLLSVSATFLPPPPRTSLSPPVSNFFTVSLAGPQGQIIGGFVAGPLISAGTVYVIAASFNNPSYHRLPAEEEQKHSAGTGEREGQSPPVSGGGEESGQMAGSGGESCGVSMYSCHMGGSDVIWAPTARAPPPY

SEQ ID NO: 47 - DNA, 798 bp, Arabidopsis thaliana
atggatgaggtatctcgttctcatacaccgcaatttctatcaagtgatcatcagcactatcaccatcaaaacgctggacgacaaaaacgcggcagagaagaagaaggagttgaacccaacaatataggggaagacctagccacctttccttccggagaagagaatatcaagaagagaaggccacgtggcagacctgctggttccaagaacaaacccaaagcaccaatcatagtcactcgcgactccgcgaacgccttcagatgtcacgtcatggagataaccaacgcctgcgatgtaatggaaagcctagccgtcttcgctagacgccgtcagcgtggcgtttgcgtcttgaccggaaacggggccgttacaaacgtcaccgttagacaacctggcggaggcgtcgtcagtttacacggacggtttgagattctttctctctcgggttcgtttcttcctccaccggcaccaccagctgcgtctggtttaaaggtttacttagccggtggtcaaggtcaagtgatcggaggcagtgtggtgggaccgcttacggcatcaagtccggtggtcgttatggcagcttcatttggaaacgcatcttacgagaggctgccactagaggaggaggaggaaactgaaagagaaatagatggaaacgcggctagggcgattggaacgcaaacgcagaaacagttaatgcaagatgcgacatcgtttattgggtcgccgtcgaatttaattaactctgtttcgttgccaggtgaagcttattggggaacgcaacgaccgtctttctaa

SEQ ID NO: 48 - protein, 265 aa, Arabidopsis thaliana
MDEVSRSHTPQFLSSDHQHYHHQNAGRQKRGREEEGVEPNNIGEDLATFPSGEENIKKRRPRGRPAGSKNKPKAPIIVTRDSANAFRCHVMEITNACDVMESLAVFARRRQRGVCVLTGNGAVTNVTVRQPGGGVVSLHGRFEILSLSGSFLPPPAPPAASGLKVYLAGGQGQVIGGSVVGPLTASSPVVVMAASFGNASYERLPLEEEEETEREIDGNAARAIGTQTQKQLMQDATSFIGSPSNLINSVSLPGEAYWGTQRPSF

SEQ ID NO: 49 - DNA, 948 bp, Arabidopsis thaliana
atggcgaatccatggtggacaggacaagtgaacctatccggcctcgaaacgacgccgcctggttcctctcagttaaagaaaccagatctccacatctccatgaacatggccatggactcaggtcacaataatcatcaccatcaccaagaagtcgataacaacaacaacgacgacgatagagacaacttgagtggagacgaccacgagccacgtgaaggagccgtagaagcccccacgcgccgtccacgtggacgtcctgctggttccaagaacaaaccaaagccaccgatcttcgtcactcgcgattctccaaatgctctcaagagccatgtcatggagatcgctagtgggactgacgtcatcgaaaccctagctacttttgctaggcggcgtcaacgtggcatctgcatcttgagcggaaatggcacagtggctaacgtcaccctccgtcaaccctcgaccgctgccgttgcggcggctcctggtggtgcggctgttttggctttacaagggaggtttgagattctttctttaaccggttctttcttgccaggaccggctccacctggttccaccggtttaacgatttacttagccggtggtcaaggtcaggttgttggaggaagcgtggtgggcccattgatggcagcaggtccggtgatgctgatcgccgccacgttctctaacgcgacttacgagagattgccattggaggaggaagaggcagcagagagaggcggtggtggaggcagcggaggagtggttccggggcagctcggaggcggaggttcgccactaagcagcggtgctggtggaggcgacggtaaccaaggacttccggtgtataatatgccgggaaatcttgtttctaatggtggcagtggtggaggaggacagatgagcggccaagaagcttatggttgggctcaagctaggtcaggattttaa

SEQ ID NO: 50 - protein, 315 aa, Arabidopsis thaliana
MANPWWTGQVNLSGLETTPPGSSQLKKPDLHISMNMAMDSGHNNHHHHQEVDNNNNDDDRDNLSGDDHEPREGAVEAPTRRPRGRPAGSKNKPKPPIFVTRDSPNALKSHVMEIASGTDVIETLATFARRRQRGICILSGNGTVANVTLRQPSTAAVAAAPGGAAVLALQGRFEILSLTGSFLPGPAPPGSTGLTIYLAGGQGQVVGGSVVGPLMAAGPVMLIAATFSNATYERLPLEEEEAAERGGGGGSGGVVPGQLGGGGSPLSSGAGGGDGNQGLPVYNMPGNLVSNGGSGGGGQMSGQEAYGWAQARSGF

SEQ ID NO: 51 - DNA, 1343 bp, Arabidopsis thaliana
ttagtatcattctttgtcgtgttcttttaattaaccttttgcaatttgtcttgtgtttctcacaacacaaaaacttgtaaaagtgttaaaaaatcaagatctgaaaaatcttatcaccgcttctaggtttttcagttttttttcttccttttcctgatctaaattaacttatatttcttagggtttcacttcttgaaacatttaatcagaattaattaacctctctagggctttcatggcgaatccatggtggacaggacaagtgaacctatccggcctcgaaacgacgccgcctggttcctctcagttaaagaaaccagatctccacatctccatgaacatggccatggactcaggtcacaataatcatcaccatcaccaagaagtcgataacaacaacaacgacgacgatagagacaacttgagtggagacgaccacgagccacgtgaaggagccgtagaagcccccacgcgccgtccacgtggacgtcctgctggttccaagaacaaaccaaagccaccgatcttcgtcactcgcgattctccaaatgctctcaagagccatgtcatggagatcgctagtgggactgacgtcatcgaaaccctagctacttttgctaggcggcgtcaacgtggcatctgcatcttgagcggaaatggcacagtggctaacgtcaccctccgtcaaccctcgaccgctgccgttgcggcggctcctggtggtgcggctgttttggctttacaagggaggtttgagattctttctttaaccggttctttcttgccaggaccggctccacctggttccaccggtttaacgatttacttagccggtggtcaaggtcaggttgttggaggaagcgtggtgggcccattgatggcagcaggtccggtgatgctgatcgccgccacgttctctaacgcgacttacgagagattgccattggaggaggaagaggcagcagagagaggcggtggtggaggcagcggaggagtggttccggggcagctcggaggcggaggttcgccactaagcagcggtgctggtggaggcgacggtaaccaaggacttccggtgtataatatgccgggaaatcttgtttctaatggtggcagtggtggaggaggacagatgagcggccaagaagcttatggttgggctcaagctaggtcaggattttaacgtgcgttaaaatggtttttaatttacagaagttaacaataagattataatgatgtttattatgatgatgaaaaccagtcagttgctacttgttactagtgagctatatagtttgtggacattatattatgttctctcttgactatgattattatttgct

SEQ ID NO: 52 - protein, 315 aa, Arabidopsis thaliana; identical to SEQ ID NO: 50

SEQ ID NO: 53 - DNA, 858 bp, Arabidopsis thaliana
atggctggtctcgatctaggcacaacttctcgctacgtccacaacgtcgatggtggcggcggcggacagttcaccaccgacaaccaccacgaagatgacggtggcgctggaggaaaccaccatcatcaccatcataatcataatcaccatcaaggtttagatttaatagcttctaatgataactctggactaggcggcggtggaggaggagggagcggtgacctcgtcatgcgtcggccacgtggccgtccagctggatcgaagaacaaaccgaagccgccggtgattgtcacgcgcgagagcgcaaacactcttagggctcacattcttgaagttggaagtggctgcgacgttttcgaatgtatctccacttacgctcgtcggagacagcgcgggatttgcgttttatccgggacgggaaccgtcactaacgtcagcatccgtcagcctacggcggccggagctgttgtgactctgcggggtacttttgagattctttccctctccggatcttttcttccgccacctgctcctccaggggcgactagcttgacgatattcctcgctggagctcaaggacaggtcgtcggaggtaacgtagttggtgagttaatggcggcggggccggtaatggtcatggcagcgtcttttacaaacgtggcttacgaaaggttgcctttggacgagcatgaggagcacttgcaaagtggcggcggcggaggtggagggaatatgtactcggaagccactggcggtggcggagggttgcctttctttaatttgccgatgagtatgcctcagattggagttgaaagttggcaggggaatcacgccggcgccggtagggctccgttttag

SEQ ID NO: 54 - protein, 285 aa, Arabidopsis thaliana
MAGLDLGTTSRYVHNVDGGGGGQFTTDNHHEDDGGAGGNHHHHHHNHNHHQGLDLIASNDNSGLGGGGGGGSGDLVMRRPRGRPAGSKNKPKPPVIVTRESANTLRAHILEVGSGCDVFECISTYARRRQRGICVLSGTGTVTNVSIRQPTAAGAVVTLRGTFEILSLSGSFLPPPAPPGATSLTIFLAGAQGQVVGGNVVGELMAAGPVMVMAASFTNVAYERLPLDEHEEHLQSGGGGGGGNMYSEATGGGGGLPFFNLPMSMPQIGVESWQGNHAGAGRAPF

SEQ ID NO: 55 - DNA, 954 bp, Arabidopsis thaliana
atggatcaggtctctcgctctcttcctccaccttttctctcaagagatctccatcttcacccacaccatcaattccagcatcagcagcagcagcagcaacagaatcacggccacgatatagaccagcaccgaatcggtgggctaaaacgtgaccgagatgctgatatcgatcccaacgagcactcttcagccggaaaagatcaaagtactcctggctccggtggagaaagcggcggcggaggaggaggagataatcacatcacgagaaggccacgtggcagaccagcgggatctaagaacaaaccaaaaccgccaatcatcatcactcgagacagcgcaaacgctctcaaatctcatgtcatggaagtagcaaacggatgtgacgtcatggaaagtgtcaccgtcttcgctcgccgtcgccaacgtggcatctgcgttttgagcggaaacggcgccgttaccaacgttaccataagacaaccagcttcagtacctggtggtggctcatctgtcgttaacttacacggacgtttcgagattctttctctctcgggatcattccttcctcctccggctccaccagctgcgtcaggtctaacgatttacttagccggtggtcagggacaggttgttggaggaagcgtggttggtccactcatggcttcaggacctgtagtgattatggcagcttcgtttggaaacgctgcgtatgagagactgccgttggaggaagacgatcaagaagagcaaacagctggagcggttgctaataatatcgatggaaacgcaacaatgggtggtggaacgcaaacgcaaactcagacgcagcagcaacagcaacaacagttgatgcaagatccgacgtcgtttatacaagggttgcctccgaatcttatgaattctgttcaattgccagctgaagcttattggggaactccgagaccatctttctaa

SEQ ID NO: 56 - protein, 317 aa, Arabidopsis thaliana
MDQVSRSLPPPFLSRDLHLHPHHQFQHQQQQQQQNHGHDIDQHRIGGLKRDRDADIDPNEHSSAGKDQSTPGSGGESGGGGGGDNHITRRPRGRPAGSKNKPKPPIIITRDSANALKSHVMEVANGCDVMESVTVFARRRQRGICVLSGNGAVTNVTIRQPASVPGGGSSVVNLHGRFEILSLSGSFLPPPAPPAASGLTIYLAGGQGQVVGGSVVGPLMASGPVVIMAASFGNAAYERLPLEEDDQEEQTAGAVANNIDGNATMGGGTQTQTQTQQQQQQQLMQDPTSFIQGLPPNLMNSVQLPAEAYWGTPRPSF

SEQ ID NO: 57 - DNA, 1379 bp, Arabidopsis thaliana
ctcaccttctccttctactttttatcatttccctttctcttcccattttagtcttcctataacttcttctcaatcctctctcatatcttttttcttagtttaaatttcaataaaatagaaaaaaacatatacaaatctacagagaagagaagctttattttaatcttgtgtgtgtgtgtgtgttttatataatttttattttttttcaaattaaaatctcttctttgcttttgatgtgggcatggctggtcttgatctaggcacagcttttcgttacgttaatcaccagctccatcgtcccgatctccaccttcaccacaattcctcctccgatgacgtcactcccggagccgggatgggtcatttcaccgtcgacgacgaagacaacaacaacaaccatcaaggtcttgacttagcctctggtggaggatcaggaagctctggaggaggaggaggtcacggcgggggaggagacgtcgttggtcgtcgtccacgtggcagaccaccgggatccaagaacaaaccgaaacctccggtaattatcacgcgcgagagcgcaaacactctaagagctcacattcttgaagtaacaaacggctgcgatgttttcgactgcgttgcgacttatgctcgtcggagacagcgagggatctgcgttctgagcggtagcggaacggtcacgaacgtcagcatacgtcagccatctgcggctggagcggttgtgacgctacaaggaacgttcgagattctttctctctccggatcgtttcttcctcctccggcacctcccggagcaacgagtttgacaattttcttagccggaggacaaggtcaggtggttggaggaagcgttgtgggtgagcttacggcggctggaccggtgattgtgattgcagcttcgtttactaatgttgcttatgagagacttcctttagaagaagatgagcagcagcaacagcttggaggaggatctaacggcggaggtaatttgtttccggaggtggcagctggaggaggaggaggacttccgttctttaatttaccgatgaatatgcaaccaaatgtgcaacttccggtggaaggttggccggggaattccggtggaagaggtcctttctgatgtgtatatattgataatcattatatatataccggcggagaagcttttccggcgaagaatttgcgagagtgaagaaaggttagaaaagcttttaatggactaatgaatttcaaattatcatcgtgatttcggacattgtcttgttcatcatgttaagcttaggtttattttttgtcgtttgtagaattttatgtttgaatccttttttttttctgtgaaactctattgtgttcgtctgcgaaggaaaaaaaaattctca

SEQ ID NO: 58 - protein, 292 aa, Arabidopsis thaliana
MAGLDLGTAFRYVNHQLHRPDLHLHHNSSSDDVTPGAGMGHFTVDDEDNNNNHQGLDLASGGGSGSSGGGGGHGGGGDVVGRRPRGRPPGSKNKPKPPVIITRESANTLRAHILEVTNGCDVFDCVATYARRRQRGICVLSGSGTVTNVSIRQPSAAGAVVTLQGTFEILSLSGSFLPPPAPPGATSLTIFLAGGQGQVVGGSVVGELTAAGPVIVIAASFTNVAYERLPLEEDEQQQQLGGGSNGGGNLFPEVAAGGGGGLPFFNLPMNMQPNVQLPVEGWPGNSGGRGPF

SEQ ID NO: 59 - DNA, 1563 bp, Arabidopsis thaliana
actttccaaatttcacttcctccaactcaaagaaagagatcagaagctatagctatacaccaaaactaggtatagagagagaaaccctacaaaatcaaaaggaagaagagacgaagatgaagaagatccagcttggatttcgttgttcatcattactactctctttcttcttctagctagctagttttgacagcaaaataagaagcaaaaaaaaggtcaactaaaaaagatctgttcttagatcactctcttcttctttttttgatccaattccaccattgaatcatagatcatggatccagtacaatctcatggatcacaaagctctctacctcctcctttccacgcaagagactttcaattacatcttcaacaacagcaacaagagttcttcctccaccatcaccagcaacaaagaaaccaaaccgatggtgaccaacaaggaggatcaggaggaaaccgacaaatcaagatggatcgtgaagagacaagcgacaacatagacaacatagctaacaacagcggtagtgaaggtaaagacatagatatacacggtggttcaggagaaggaggtggtggctccggaggagatcatcagatgacaagaagaccaagaggaagaccagcgggatccaagaacaaaccaaaaccaccgattatcatcacacgggacagcgcaaacgcgcttagaacccacgtgatggagatcggagatggctgcgacttagtcgaaagcgttgccacttttgcacgaagacgccaacgcggcgtttgcgttatgagcggtactggaaatgttactaacgtcactatacgtcagcctggatctcatccttctcctggctcggtagttagtcttcacggaaggttcgagattctatctctctcaggatcttttctccctcctccggctcctcctacagccaccggattgagtgtttacctcgctggaggacaaggacaggtggttggaggaagcgtagttggtccgttgttatgtgctggtcctgtcgttgtcatggctgcgtcttttagcaatgcggcgtacgaaaggttgcctttagaggaagatgagatgcagacgccggttcatggcggaggaggaggaggatcattggagtcgccgccaatgatgggacaacaactgcaacatcagcaacaagctatgtcaggtcatcaagggttaccacctaatcttcttggttcggttcagttgcagcagcaacatgatcagtcttattggtcaacgggacgaccaccgtattgatcaaatatacacacacactcataatcgttgctagctagctaacgatgaatcatgagtttagtggatatatatatgattaaaagaggttagcttatgaacattaataagagtttggattctatcgagcttcattatgtttgggtcaacgttcataagatgaaccaggttcttttttctcaaatttttattttggttcgtttttagctcgtagatctttttatgttgattttagtaacatttttctgtttgtcttggtttttctttgatgaattctcattgctagtttgcatttgatt

SEQ ID NO: 60 - protein, 324 aa, Arabidopsis thaliana
MDPVQSHGSQSSLPPPFHARDFQLHLQQQQQEFFLHHHQQQRNQTDGDQQGGSGGNRQIKMDREETSDNIDNIANNSGSEGKDIDIHGGSGEGGGGSGGDHQMTRRPRGRPAGSKNKPKPPIIITRDSANALRTHVMEIGDGCDLVESVATFARRRQRGVCVMSGTGNVTNVTIRQPGSHPSPGSVVSLHGRFEILSLSGSFLPPPAPPTATGLSVYLAGGQGQVVGGSVVGPLLCAGPVVVMAASFSNAAYERLPLEEDEMQTPVHGGGGGGSLESPPMMGQQLQHQQQAMSGHQGLPPNLLGSVQLQQQHDQSYWSTGRPPY

SEQ ID NO: 61 - DNA, 1505 bp, Arabidopsis thaliana
agacaagagtagagagagaaaataaaaccttaccaaatcaatcaaagaataagatgaagaacaaaattccagcttgatgtattttgtgaaaacattattattcttttcctcttctatctagagtctctcctaggtcaaattatcaaaatcaattctcatctcaacatagaatggatccagttcaatctcatggatcacaaagctctcttcctcctcctttccatgctagagatttccaattacatcttcaacaacaacaacaacatcaacaacaacatcaacaacaacaacaacaacagttctttctccaccatcatcagcaaccacaaagaaaccttgatcaagatcacgagcagcaaggagggtcaatattgaatagatctatcaagatggatcgcgaagagacaagcgataacatggacaacatcgctaataccaacagcggtagcgaaggtaaagagatgagtttacacggaggagaaggaggaagcggtggtggaggaagtggagaacagatgacaagaaggccaagaggaagaccagcaggatccaagaacaaacctaaagctccaataatcataacaagagacagcgcaaacgcgcttcgaactcacgtcatggagataggagacggatgtgacatagttgactgtatggctacgttcgctagacgccgccaaagaggcgtttgcgttatgagcggtacaggaagcgttactaacgtcactatacgtcagcctggatcgccacctggctcggtggttagccttcacggccggtttgaaatcctctctctttcgggatctttcttgcctccgcctgcgccgcctgcagccaccggactaagcgtttacctagccggaggacaagggcaggtcgttggaggtagtgtggtgggacctttgttgtgttcgggtcctgtggtggttatggcggcttcttttagcaatgcggcgtacgaaaggctgcctttggaagaagatgagatgcagacgccagttcaaggaggcggtggaggaggaggaggtggtggtggaatgggatctcccccgatgatgggacagcaacaagctatggcagctatggcggcggctcaaggactaccaccgaatcttcttggttcggttcagttgccaccgccacaacagaatgatcagcagtattggtctacgggtcggccaccgtattgattattagattgctattattatatatacatgatcaatggaatatatacatatatagctatgtatttgtatatatgtagctatttactaatagatcttacatatatataaagcaagctaacatggaatcattaaaaggaggtctacgaagtatgacgtgaagttgaagacggcgataatattgaagatcaacaattcagtagatggttttcaggttcttctttgttttttttgtttcctttgggttcgttttagtggtagatcttttttttttaatgtttgattatgtgaggttaatttaagttgttatttacttta

SEQ ID NO: 62 - protein, 339 aa, Arabidopsis thaliana
MDPVQSHGSQSSLPPPFHARDFQLHLQQQQQHQQQHQQQQQQQFFLHHHQQPQRNLDQDHEQQGGSILNRSIKMDREETSDNMDNIANTNSGSEGKEMSLHGGEGGSGGGGSGEQMTRRPRGRPAGSKNKPKAPIIITRDSANALRTHVMEIGDGCDIVDCMATFARRRQRGVCVMSGTGSVTNVTIRQPGSPPGSVVSLHGRFEILSLSGSFLPPPAPPAATGLSVYLAGGQGQVVGGSVVGPLLCSGPVVVMAASFSNAAYERLPLEEDEMQTPVQGGGGGGGGGGGMGSPPMMGQQQAMAAMAAAQGLPPNLLGSVQLPPPQQNDQQYWSTGRPPY

SEQ ID NO: 63 - DNA, 621 bp, Arabidopsis thaliana
atggaaaccgtcgggcgtccacgtggcagacctcgaggttccaaaaacaaacctaaagctccaatctttgtcaccattgaccctcctatgagtccttacatcctcgaagtgccatccggaaacgatgtcgttgaagccctaaaccgtttctgccgcggtaaagccatcggcttttgcgtcctcagtggctcaggctccgttgctgatgtcactttgcgtcagccttctccggcagctcctggctcaaccattactttccacggaaagttcgatcttctctctgtctccgccactttcctccctcctctacctcctacctccttgtcccctcccgtctccaatttcttcaccgtctctctcgccggacctcaggggaaagtcatcggtggattcgtcgctggtcctctcgttgccgccggaactgtttacttcgtcgccactagtttcaagaacccttcctatcaccggttacctgctacggaggaagagcaaagaaactcggcggaaggggaagaggagggacaatcgccgccggtctctggaggtggtggagagtcgatgtacgtgggtggctctgatgtcatttgggatcccaacgccaaagctccatcgccgtactga

SEQ ID NO: 64 - protein, 206 aa, Arabidopsis thaliana
METVGRPRGRPRGSKNKPKAPIFVTIDPPMSPYILEVPSGNDVVEALNRFCRGKAIGFCVLSGSGSVADVTLRQPSPAAPGSTITFHGKFDLLSVSATFLPPLPPTSLSPPVSNFFTVSLAGPQGKVIGGFVAGPLVAAGTVYFVATSFKNPSYHRLPATEEEQRNSAEGEEEGQSPPVSGGGGESMYVGGSDVIWDPNAKAPSPY

SEQ ID NO: 65 - DNA, 2059 bp, Oryza sativa
ggcattgtatcgtgagagagattgatagagagagaggcggacctccctgaccttatcggcagcgccatcccatcccttgccgtccccccaagagctcacccctccgcctcgcttcttcatctcctctcctttaattcggccccgcgccgcccgcttcacccccatcctacaccgcgtgcctccttaccttctctttgttggaataacgatcgtttcgcttgctgctccggggaggtttttcttgggcgatcgatcggctgggcaggttcgtcggagggaggtttctgccgcgcgccgccggggtgtcgacgtgtcgagcggtgcgtggaggaggagaagcgtgcggcggaggttgtggtggtgatggtgaggagcgagttgtagaggaggaggggcaccgatcaaccgggaagctggcggccgggaagtggtgggagcggccagatgacacggccatggccgggatggaccctggcgggggcggcgccggcgccggcagctcacggtacttccaccatctgctccgaccgcagcagccgtcgccgctgtcaccgctgtcgccgacatcccatgtcaagatggagcactccaagatgtcacccgacaagagccccgtgggcgagggagatcacgcgggagggagtggaagcggcggcgtcggcggtgaccaccagccgtcgtcgtcggccatggtgcccgtcgagggtggcagcggcagcgccggcggtagtggctcgggtgggccgacgcggcgcccgcgcgggcgcccgcccgggtccaagaacaagccgaagccgcccatcatcgtgacgcgcgacagcccgaacgcgctgcactcgcacgtgctcgaggtcgccggcggcgccgacgtcgtcgactgcgtggccgagtacgcccgccgccgagggcgcggcgtgtgcgtgctgagcggcggcggcgccgtcgtcaacgtggcgctgcggcagccgggcgcgtcgccgccgggcagcatggtggccacgctgcggggccggttcgagatcctatctctcacgggcacggtcctgccgcctcccgcgccacccggcgcgagcggcctcaccgtgttcctctccggcggccagggccaggtgatcggcggcagcgtggtgggcccgctggtcgccgcggggcccgtcgtcctgatggcggcctcattcgcgaacgccgtgtacgagcggctgccgctggagggcgaggaagaggaggtcgccgcgcccgccgccggaggcgaagcacaagatcaagtggcacaatcagctggacccccagggcagcaaccggcggcgtcacagtcctccggcgtgacaggaggcgacggcaccggcggcgccggtggcatgtcgctctacaacctcgccgggaatgtgggaggctatcagctccccggagacaacttcggaggttggagcggcgccggcgccggcggagtcaggccaccgttctgacccatgtcttagcatccagttcaaaaattctccaaattaagaattgcgcagtgcagaagccatagcaatccaatgcagatatgcaagctaagctaatggtacaaaattgcgacatgcatgttggattgttgattggacgatcgacaggcctttgtactgggtgcaacatacgtatgcatctatcatgcatgcgttgcaaattaagatttcaattcatattcatgaggaggtttatcatatacacatattttaacatctgtgggggcaagaagctagaggagagttgcaagagcaagctcggaataaaccaaaggtcagtgagattatccaatgttttgtgtttaattctaccaagtgtttttttttcatttttttctcttttgcattctgtaatatggtactatcctattctagtttgtatcttttgatcggtagaaccatgtttctgaaaaactcttgttcccttttttcaacctctgaaacatgtgcaacaccagaactgtcttatggtcactttcactggtctgaattcaagctcacgatttggtggcgttgaatattattttttatttttattttttgttttggaacca

SEQ ID NO: 66 - protein, 336 aa, Oryza sativa
MAGMDPGGGGAGAGSSRYFHHLLRPQQPSPLSPLSPTSHVKMEHSKMSPDKSPVGEGDHAGGSGSGGVGGDHQPSSSAMVPVEGGSGSAGGSGSGGPTRRPRGRPPGSKNKPKPPIIVTRDSPNALHSHVLEVAGGADVVDCVAEYARRRGRGVCVLSGGGAVVNVALRQPGASPPGSMVATLRGRFEILSLTGTVLPPPAPPGASGLTVFLSGGQGQVIGGSVVGPLVAAGPVVLMAASFANAVYERLPLEGEEEEVAAPAAGGEAQDQVAQSAGPPGQQPAASQSSGVTGGDGTGGAGGMSLYNLAGNVGGYQLPGDNFGGWSGAGAGGVRPPF

SEQ ID NO: 67 - DNA, 2015 bp, Oryza sativa
ttctcctcctccaccttgccactactactactaccttttccttgagcggcgccgcgcccccgcccccgccctgccggccaacgagatcgatcgactatttgcgccgccgccgccgcctcctccttcccctggacacgtggcccgcccgagacgtcaaagaggaagaagacgaagaagcttcgctccgaccaggggtgaggtgaggtgaggggtgggatcgagggatcgagaagctggcggcgagcaagtggtgggagcgcgccgatgagggcgccatggccgggatggatcccaccggcggcggtggcggcggcggcgtggcggcgcactacctacacatgctccgcgcgcagcagcaccagccactgtccccggcaggtgacgtcaaggcggagcggtccatgctgtcgccggatgagagccccggcgcggacgccgacctaggatcggaccacccgacgtcgtcggccatggtggcggcggaggacagcggcggcggcagcggttcgggtggcccgatgcggcgcccccgcgggaggccgctgggctccaagaacaagcccaagccgcccatcatcgtgacgcgggacagccccaacgcgttccactcccacgtcctcgaggtcgccgcgggaaccgacatcgtcgagtgcgtctgcgagttcgcgcgccgccgcggccgcggcgtctccgtgctcagcggtggcggcgccgtcgccaacgtcgcgctccgccagccaggcgcgtcgcccccgggcagcctggtcgccaccatgcgcggccagttcgagatcctgtccctcacgggcaccgtcctcccgccgcccgcgccgcccagcgccagcggcctcaccgtcttcctctccggcgggcagggccaggtggtcggcgggagcgtggccggccagctcatcgccgcggggccagtcttcctcatggccgcctcgttcgccaatgccgtctacgagcgtctgccactcgatggggaggatccggaggcagaggctgccgccgccacccctcccggcgatgcggcgcagccaaccggcccaccaccaccgcagcagcagcccacagcctcgcagtcctctgaggtgaccgccggtgacggcggcggcggcggcggtctcggcatgtatcttggaggccatgtgggatcctaccagcagcagcagcagcaacttcccggaccaggagacaacttcggtagctggagcggcagcatcaggccgccgccattctgatccaaacacctcaaatcaagctctcccccaacaacgccatgcatgtctaaatcctcacaagattcactccaagaagacgaagctgagtgagagtgatggatttcatttcatctcatcatttgtgacaatgcattcatttttttttgttgttgaaggctatacatcttgcaagattacaagttaatcgaggcgcatgcattcttgattggagatgtggaagaatctcttgcattgcagaagtactaatagaatcaacattggaaggcagaattttttttggctggagatggagggaggtcagtcaattagtgcaaggaactgaacaagatcccggtaaggcaggttagtaggaccccggtgtttagtggttcatcctgtagacttttgatcaagaatgatgcttgtcatgaccttttacttaaaaactttcccttggaatgatgcttgttattgcttttcactacaactccactctttcttgactgctactaccagtttttttacacctttttcaccgttgcacatttgcacttgtcctgcttcctggcaccacggtaaaaaatctgcaaactagtgattcgtctcattcgaactgtactccaaggagagctgtcttccagccttctttttggtgtcatttccacacattggaaatcacctgcttttggtcaattttgtcttttctttgagccagtggagagtagagtgaccaccactgaccagagctgtaaacatttcaaaccctagcttgggggccctttggatgcgtta

SEQ ID NO: 68 - protein, 322 aa, Oryza sativa
MAGMDPTGGGGGGGVAAHYLHMLRAQQHQPLSPAGDVKAERSMLSPDESPGADADLGSDHPTSSAMVAAEDSGGGSGSGGPMRRPRGRPLGSKNKPKPPIIVTRDSPNAFHSHVLEVAAGTDIVECVCEFARRRGRGVSVLSGGGAVANVALRQPGASPPGSLVATMRGQFEILSLTGTVLPPPAPPSASGLTVFLSGGQGQVVGGSVAGQLIAAGPVFLMAASFANAVYERLPLDGEDPEAEAAAATPPGDAAQPTGPPPPQQQPTASQSSEVTAGDGGGGGGLGMYLGGHVGSYQQQQQQLPGPGDNFGSWSGSIRPPPF

SEQ ID NO: 69 - DNA, 1310 bp, Oryza sativa
ggaggaaaaagaaagcaagtgagtggagtgaaagagagagaggaagagcaagagggataacctagctaattgattgctagcttgcaaggatggatccggtcacggcatcaatacacggtcaccatcttcctccaccgttcaacacccgcgacttccatcaccatctccagcagcagcagcaccagctgcatctcaagaccgaggatgaccaaggcggcggcactccgggtgtcttcggcagccgcggcaccaagcgcgaccacgacgacgacgagaacagtggcaacggccatggaagcggtggtgacggcggtgacctcgcgctggtacccccctcgggtggcgggccggacggcgccgggagcgagagcgccacgcgccgcccgaggggacgcccggcggggtccaagaacaagccgaagccaccgatcatcatcaccagggacagcgccaacacgctccggacgcacgtcatggaggtggccggcggctgcgacatctccgagagcatcaccacgttcgcgcgacgccggcagcgcggggtttgcgtgctcagcggcgccggcaccgtcactaacgtcacgctgcggcagcccgcatcgcagggagcggtcgttgcgctccacggccggttcgagatactctccctctccggctccttcctcccgccgcccgccccgccggaggccacggggctcaccgtctacctggccggaggccagggccaggtcgtgggcggcagcgtcgtcggcgcgctgaccgcggctgggcctgtggtgataatggcggcgtcttttgcgaacgcggtgtacgagcggctgccgttggaggacgacgagctactggcggctcaagggcaagccgacagcgctgggttgctcgccgcggggcagcaagcggcgcagctcgccggcggggccgtcgatccaagcctcttccaaggactaccaccaaacctactcggaaacgtgcagctgccgccggaagccgcctacggatggaaccctggagccggcggtggccgcccggcgccgttctgagatggatcgattccgcgacagcaacgcagcatagaaaggttaagtgctttaattagctttctcttcacttggagttggagactactctttcttggaggatttccgtgggaattatgcatgtgatttcaggtgttattaatcgttattatgtcagcgtcggggattaattgtttcgtgatatgtagctctctctctctctctctctgtctctgtctttttttcggctatttcttgtgtcttcctcttaactgatctcgctgttgactcgag

SEQ ID NO: 70 - protein, 316 aa, Oryza sativa
MDPVTASIHGHHLPPPFNTRDFHHHLQQQQHQLHLKTEDDQGGGTPGVFGSRGTKRDHDDDENSGNGHGSGGDGGDLALVPPSGGGPDGAGSESATRRPRGRPAGSKNKPKPPIIITRDSANTLRTHVMEVAGGCDISESITTFARRRQRGVCVLSGAGTVTNVTLRQPASQGAVVALHGRFEILSLSGSFLPPPAPPEATGLTVYLAGGQGQVVGGSVVGALTAAGPVVIMAASFANAVYERLPLEDDELLAAQGQADSAGLLAAGQQAAQLAGGAVDPSLFQGLPPNLLGNVQLPPEAAYGWNPGAGGGRPAPF

SEQ ID NO: 71 - DNA, 1668 bp, Sorghum bicolor
cttcctccagaaccttgaccgaccccaagagctttttgcttgggcggcgccgcgccgcaacacgccgcgccggccaagatttagttggagcgcaagcaaggatgctggatgcatgatgcgcccgtgcgctccgacctctagatcgcccccacactcctgctgcagctgctactggtagagcactccacgggtgaggggatccgccaagcagacagtccaaataaagaacgagctggttcaagcagctacgttgggacgagcactagcgagatcaagaagctggcggcgagcaagtggtgggagcgcggcggtgagggcgtcatggccggaatggatcctggcggcggcggcgggactgcgtcgcactacctggaactcctccgcgcgcagcagctgcagcaccagcagccgtcggcgccgctatccccttcctcccacgtcaagatggagcgctccgcgccgtcgccggagaacgtcgatcccggcggggatcagcccgccttggagggcagcggggggtccggtgggccgatgaggaagccgcgcgggcggccgccggggtccaagaacaagcccaagccgcccatcatcataacgcgggacagccccaacgcgctccactcccacgtcctcgaggtcgccgccggcgccgacatcgtcgagtgcgtctcggagtacgcgcgccgccgctgccgcggcgtatgcgtgctcagcggcggcggcgccgtctccaacctcgcgctacgccagcccggcgccgagcccccgggcagcctcgtcgccaccctgcgcgggcagttcgagatcctgtcgctcacgggcacggtgctgccgccgcccgcgcccccgggagccagcagccttagcgtgtacgtcgccggcgggcaggggcaggtgatgggcgggagcgtggtcggccagctcatcgccgccggacccgtagtcctcatggccgcgtcgttcgcgaacgccgtctacgagcggctgccgctggaggctgaggaggaagaggcggccacggcagccgcagccgctgctgctgccacagagacccaaggcgcagcggagccggcggaaggacagccacagcaacaggaggcgtcgcagtcttctggtgtgacgggtggcgatggcggtggcggtggcattggccatggcatgtcgctgtatgatcttggagggaacgcggctggctaccagttgcccggagagaacttcggcacctggagcggtggtatgaggccgccattttgataccaatctctttctcttggcaattgcatttgcatcacggcaagtttgagaagatcccgatccatgacagagaagctccttaatcctgcatttcggttgaaggctacactttgacgagggcacggaggccatgtgagatgatttgctgagaatgcatgctgccatggtggtcgactggaaataatccagatccatgtatacttataccatctatgactatggaaaagtgagtactaccgacgatgcatgtctattttttgggtgatctattagtccaacaaaatccaaggaaggtcagtaggtccagtgttaactgtttagagaccatccctgtcgacttgtccttttgctttagaacgatctttgtcaagttgtcatgacatttgcctttttaatagtacttgcgttctttct

SEQ ID NO: 72 - protein, 310 aa, Sorghum bicolor
MAGMDPGGGGGTASHYLELLRAQQLQHQQPSAPLSPSSHVKMERSAPSPENVDPGGDQPALEGSGGSGGPMRKPRGRPPGSKNKPKPPIIITRDSPNALHSHVLEVAAGADIVECVSEYARRRCRGVCVLSGGGAVSNLALRQPGAEPPGSLVATLRGQFEILSLTGTVLPPPAPPGASSLSVYVAGGQGQVMGGSVVGQLIAAGPVVLMAASFANAVYERLPLEAEEEEAATAAAAAAAATETQGAAEPAEGQPQQQEASQSSGVTGGDGGGGGIGHGMSLYDLGGNAAGYQLPGENFGTWSGGMRPPF

SEQ ID NO: 73 - DNA, 987 bp, Sorghum bicolor
atgccgggaatggacccgggcgggggcggcagctcgcgctacttccaccagctgctgcggccgcagcagcagcagcagccgtcgccgctgtcgcccaattcccacgtcaagatggagcaccaccacaagatgtctccggacaagagccccgtcggtggcgaggccgaggcgggcgggagcggcggcggcggcgaccagccgtcctcgtcggctttggtcccagtcgagggtggcagcggcgggggaggaggcagcgggtcgggcacgcccacgcggcggccgcggggccgcccgccggggtccaagaacaagcctaagccgcccatcatcgtgacgcgcgacagccccaacgcgctgcactcgcacgtcctcgaggtcgccgcgggagccgacgtcgtggactgcgtcgcggagtacgcgcgccgccggggtcgaggcgtgtgcgtgctcagcggcgggggcgccgtcgtcaacgtggcgctcaggcagcccggcgcgtcgccccccgggagcatggtggccacgctgaggggacggttcgagatcctctccctcaccggcaccgtgctgccgccgcccgctccccccggcgcgagtggcctcacggtgttcctctccggcgggcagggccaggtcatcggcgggagcgtggtgggcccactggtcgccgcggggcccgttgtcctgatggctgcgtcgttcgccaacgccgtgtacgagcggctcccgctggagggggaggaagaggagacagccgcggctgccgccggagccgaaccacaagatcaagtggcgcagtcggctggacccccaggtcagcagccgacggcgtctcagtcctctggtgtgaccggaggaggcgacgccggcggcggcggcatgtcgctctacaacctcgctgggaatgtaggggcctaccagctaccgggagacaacttcggaggctggagtggaggcggtggcggcggagtccggccaccgttttga

SEQ ID NO: 74 - protein, 328 aa, Sorghum bicolor
MPGMDPGGGGSSRYFHQLLRPQQQQQPSPLSPNSHVKMEHHHKMSPDKSPVGGEAEAGGSGGGGDQPSSSALVPVEGGSGGGGGSGSGTPTRRPRGRPPGSKNKPKPPIIVTRDSPNALHSHVLEVAAGADVVDCVAEYARRRGRGVCVLSGGGAVVNVALRQPGASPPGSMVATLRGRFEILSLTGTVLPPPAPPGASGLTVFLSGGQGQVIGGSVVGPLVAAGPVVLMAASFANAVYERLPLEGEEEETAAAAAGAEPQDQVAQSAGPPGQQPTASQSSGVTGGGDAGGGGMSLYNLAGNVGAYQLPGDNFGGWSGGGGGGVRPPF

SEQ ID NO: 75 - DNA, 1542 bp, Zea mays
aggaaaagtagcaaaagaatttgaagagggattgaggggggaggggagctgtctccttcgatctcgttcttgcctttttgcctctctcgaggtcagcttttggtggactcagcaaaacaaatggtaaaagcttctctgtgatctttgctcttacttgcttaattcatcaattctttggcccaatcgaactctagtgttctcttctcgttcagcctcctatatatgctcccttctttggtcaaaacatggtgcttagatctcttgacacatggctagctatgtttctcctgtattttgttttgcatctgcaaaggcgtagagcggggagcaagagattacgcacgccgcgggcaccgatttccttcaagaatctgcaagaagcggaggatctgtcgagtccgactgttctaagaagcagctcgattctcgaaaccctagctagcaaagtagcaagctagctacaagagaaggaagctggcgaacaggtggtgggacgaggggcggctcggcctgccggagccatccctaggcaagaaccaagaagaaatggcgccttcgtccaaggacggcgccaccgagcagccgacgagcggtgggagcggcgacgaccgggagaacggcaccggtgagcccaaggaaggtgcggtggtcacgggcaaccggcggccgcgcgggcggccgccgggttccaagaacaagcccaagccacccatcttcgtgacacgtgacagccccaacgcgctgcgcagccacgtgatggaggtggccggcggggccgacgtcgccgagtccatcgcccacttcgcgcgccgcaggcagcgcggcgtctgcgtgctcagcggcgccggcaccgtcaccgacgtcgcgctccgccagcccaccgcgccgggcgccgtggtcgccctccgcggccgcttcgagatcctctcgatcaccggcacgttcctgccgggacccgcgccgccgggctccacggggctgaccgtgtacctcgcgggcggccaggggcaggtcgtcggcggcagcgtggtcggcacgctcatcgcggcgggccccgtcatggtgatggcgtccacgttcgccaacgccacctacgagaggctgccgctggacgacgccgaggaggatccagggcaggcgcagctgcctcccggccccggcggaggcccccctctgataatgggcgggatagccgatccctcggcgatgccaatgttcggcggcggcggcggcggtgtgccgccaagcctgatgatgccaggaggcgccgccgcctccggtgcgggcctgcagctcgggcacgaggggcttgcttgggctcgtgcacggccaccgccatactaggaggggatccatgtctgagaaattcgagtagaaatggaggatcgttcatcgcaccggcagcctcaaggacgactgcgactcatcatatagttacttcaagctagaactgatgatcgaattaagcatgctgccaggagcaaataaaaggttgttcgactagctaaaaaaaaaaaaaaaaaaacaaaaaaaaaaaaaaaa

SEQ ID NO: 76 - protein, 265 aa, Zea mays
MAPSSKDGATEQPTSGGSGDDRENGTGEPKEGAVVTGNRRPRGRPPGSKNKPKPPIFVTRDSPNALRSHVMEVAGGADVAESIAHFARRRQRGVCVLSGAGTVTDVALRQPTAPGAVVALRGRFEILSITGTFLPGPAPPGSTGLTVYLAGGQGQVVGGSVVGTLIAAGPVMVMASTFANATYERLPLDDAEEDPGQAQLPPGPGGGPPLIMGGIADPSAMPMFGGGGGGVPPSLMMPGGAAASGAGLQLGHEGLAWARARPPPY

SEQ ID NO: 77 - DNA, 864 bp, Brassica rapa
atggacggtggttatgatcaatccggtcactctagatacttccataacctctttaggcctgagcttcaacaccagcttcagccacagccgcagcctcaaccccagcctcagcctcagcctcagcctcagtctgatgatgaatctgactccaacaacaagtatccgggtcaacctgattccgaccaggttacctcgggctcaacttccgggaagcgtccacgtggacgtcctccagggtctaagaacaagccgaagccaccggtgatagtgacaagagatagccccaacgtgcttagatctcatgttcttgaagtctcatctggagccgacataattgagagcgtcaacaattatgctcgccggagagggagaggtgtctccattctcagtggtaacggcacggtggctaacctcactctccggcagccggtgacgactcatgggaacaatggtggaactgaagccggagccggaggagttgtgactttacgtggaaggtttgagattctttccatcactggtacggtgcttccgccgcccgcgccgccgggatgcggtggtttatctatctttgttgctggtgaacaaggtcgggtgatcggaggaagagtggtggctccccttgtggcttctggtccagtgatactgatggctgcatcgttctccaacgcaactttcgaaaggcttccacttgaagaggagggaggtgaaggtgggggagacgtcggaggaggagttccaccgccagccacttcagaaacagcgccgtctggagtcgctcagggagagctaagagttaatatgagtggttatgatcagttttccggctggggagccggagccgcttcaagaccatcattttag

SEQ ID NO: 78 - protein, 287 aa, Brassica rapa
MDGGYDQSGHSRYFHNLFRPELQHQLQPQPQPQPQPQPQPQPQSDDESDSNNKYPGQPDSDQVTSGSTSGKRPRGRPPGSKNKPKPPVIVTRDSPNVLRSHVLEVSSGADIIESVNNYARRRGRGVSILSGNGTVANLTLRQPVTTHGNNGGTEAGAGGVVTLRGRFEILSITGTVLPPPAPPGCGGLSIFVAGEQGRVIGGRVVAPLVASGPVILMAASFSNATFERLPLEEEGGEGGGDVGGGVPPPATSETAPSGVAQGELRVNMSGYDQFSGWGAGAASRPSF

SEQ ID NO: 79 - DNA, 936 bp, Brassica rapa
atggaaggcggctacgagcaaggcggtggagcttctaggtacttccataacctcttcagaccagagattcaccaccaacagcttcaacaacaaggcgggatcaatctttttgaccagcatcatcaacagcaacaacatcagcagcaacaacaacaacaaccgtcagatgattcaagagaatccgatcactcaaacaaggatcatcatcaaccgggtctacccgattcagacccggctacatcaagctcagcacctgggaaacgtccacgtggacgtccaccgggatctaagaacaaagctaagccaccgatcatagtgacgcgggacagccccaatgcgcttagatctcacgtccttgaagtatctcctggagctgacatagttgagtgtgtgtccacttacgctaggcggagagggagaggggtctccgttttaggaggaaacggcaccgtttccaacgtcactcttcgtcagccagtcactcccggaaatagcggtggtggagccggaggaggagttgtgactttacatggaaggtttgagattctttcgctaacgggaaccgttttgccaccacctgcaccgccaggtgctggtggtttgtcaatatttttatccggagggcaaggtcaggtggttgggggaagcgttgtggctccgcttgttgcatcagctccggttatactaatggctgcttccttctcaaacgcggttttcgagagattgcctattgaagaggaggaagaaagaggtggtggcggtgtaggagaaggagaaggaccaccgcagatgcagcaagctccatcaccatctccgcggtcgggggtgaccggtcaaggacagctaggaggtaatgtgggtggttatgggttttccagtgatcctcatttgctaggatggggagctggtacgccttcaagaccaccttttacttaa

SEQ ID NO: 80 - protein, 311 aa, Brassica rapa
MEGGYEQGGGASRYFHNLFRPEIHHQQLQQQGGINLFDQHHQQQQHQQQQQQQPSDDSRESDHSNKDHHQPGLPDSDPATSSSAPGKRPRGRPPGSKNKAKPPIIVTRDSPNALRSHVLEVSPGADIVECVSTYARRRGRGVSVLGGNGTVSNVTLRQPVTPGNSGGGAGGGVVTLHGRFEILSLTGTVLPPPAPPGAGGLSIFLSGGQGQVVGGSVVAPLVASAPVILMAASFSNAVFERLPIEEEEERGGGGVGEGEGPPQMQQAPSPSPRSGVTGQGQLGGNVGGYGFSSDPHLLGWGAGTPSRPPFT

SEQ ID NO: 81 - protein, 289 aa, Vitis vinifera
MAGMEQGAGSRYIHQLFRPELQLERTPQQPHQPPQLNDSGDSPENEDRTDPDGSPGAATTSSRRPRGRPPGSKNKAKPPIIITRDSPNALRSHVLEISAGADIVESVSNYARRRGRGVCILSGGGAVTDVTLRQPAAPSGSVVTLHGRFEILSLTGTALPPPAPPGAGGLTIYLGGGQGQVVGGRVVGPLVASGPVLLMAASFANAVYDRLPLEEEEESPVQVQPTASQSSGVTGGGGQLGDGGNGSTTTAGGGAGAGVPFYNLGPNMGNYPFPGDVFGWNGGATRPPF

SEQ ID NO: 82 - protein, 292 aa, Vitis vinifera
MEGYEPGSGSRYVHQLLGPELHLQRPSSLPQHQATQQPSDSRDESPDDQEQRADTEEAAAASSGGATTSSNRRPRGRPPGSKNKPKPPIIVTRDSPNALRSHVLEVAAGADVMESVLNYARRRGRGVCVLSGGGTVMNVTLRQPASPAGSIVTLHGRFEILSLSGTVLPPPAPPSAGGLSIFLSGGQGQVVGGSVVGPLMASGPVVLMAASFANAVFERLPLEEEEGAVQVQPTASQSSGVTGGGAGGQLGDGGGSGGGAGVPIYNMGASMGNFPFPGDLLRWGGSAPRPPF

(19) United States
(12) Patent Application Publication
(10) Pub. No.: US 2012/0266268 A1
(43) Pub. Date: Oct. 18, 2012

(54) GENETIC MANIPULATION OF THE AT-HOOK DOMAIN IN PLANT AHL GENES TO MODULATE CELL GROWTH

(75) Inventor: Michael Neff, Pullman, WA (US)

(73) Assignee: WASHINGTON STATE UNIVERSITY, Pullman, WA (US)

(21) Appl. No.: 13/498,054

(22) PCT Filed: Sep. 27, 2010
(86) PCT No.: PCT/US10/50440

§ 371 Date: Mar. 23, 2012

Related U.S. Application Data
(60) Provisional application No. 61/246,085, filed on Sep. 25, 2009.

(51) Int. Cl. A01H 5/00; C07K 14/415; C12N 5/04; C12N 5/10; A01H 1/02; C12N 15/29; C12N 15/82

(52) U.S. Cl. 800/260; 536/23.6; 530/370; 800/290; 435/419; 800/298; 435/412; 800/320.1; 800/320.2; 800/320; 800/306; 435/410

(57) ABSTRACT

Provided are methods for generating modified plants, seedlings or seeds, comprising introducing into, or engineering in a plant cell, a nucleic acid encoding a mutant AHL protein having a mutation of the AT hook domain that confers a dominant negative phenotype as disclosed herein. Nucleic acids encoding a polypeptide comprising SEQ ID NO:3, SEQ ID NO:6, a polypeptide having at least 93% or at least 95% sequence identity with SEQ ID NO:3, or a polypeptide having at least 75% or at least 80% sequence identity with SEQ ID NO:6 are provided, along with such polypeptides having a mutation of the AT hook domain that confers a dominant negative phenotype as disclosed herein. In particular aspects, the polypeptide lacks the AT hook domain thereof. In certain aspects, the polypeptide comprises an intact or functional PPC domain, and preferably additionally comprises the linker region between the PPC domain and the AT-hook domain.

### CROSS-REFERENCE TO RELATED APPLICATIONS

This application claims the benefit of priority to U.S. Provisional Patent Application Ser. No. 61/246,085, filed 25 Sep. 2009 and entitled “GENETIC MANIPULATION OF THE AT-HOOK DOMAIN IN PLANT AHL GENES TO MODULATE CELL GROWTH,” which is incorporated herein by reference in its entirety.

### STATEMENT REGARDING FEDERALLY-SPONSORED RESEARCH

This invention was made with government support under Contract No. DE-FG02-08ER15927 awarded by the Department of Energy (DOE). The Government has certain rights in the invention.

### FIELD OF THE INVENTION

Particular aspects relate generally to modulation of cell growth in plants and plant parts, and in particular to compositions and methods comprising the use of plant (e.g., Camelina) derived AHL genes and gene products for modulation of cell growth in plants. Particular aspects relate to manipulation of the AT-hook domain in plant (e.g., Camelina) AHL genes, including manipulation of the AT-hook domain (e.g., AT-hook domain mutants and modifications including but not limited to nonsense, missence, deletions, substitutions, muteins, fusions, etc.) in novel exemplary sequences SEQ ID NOS:1-6, which have substantial utility for modulation of cell growth in plants. Additional aspects relate to modified plants, cells, or seeds comprising modified AHL genes (e.g., modified Camelina derived AHL genes) and gene products, and modified versions thereof.

### BACKGROUND

The AT-hook motif nuclear localizing gene family. The Arabidopsis thaliana genome encodes 29 AHL gene members that are characterized by containing two conserved structural elements, the AT-hook motif and the PPC domain. These 29 AHL gene members have further evolved into two phylogenic clades (Street et al. 2008, FIG. 1). Clade I consists of intron-containing AHL genes with either one or multiple AT-hook motifs and a single PPC domain. Clade II members are intron-less with only a single AT-hook motif and PPC domain (Fujimoto et al., 2004; Street et al., Plant J, doi: 10.1111/j1365-313X.2007.03393.x (2008), hereby incorporated by reference in its entirety; FIG. 2). The Clade II genes SOB3 and ESC were initially characterized with previous DOE support (Street et al. 2008), as well as family members such as HRC.

The PPC Domain. The PPC domain consists of approximately 130 amino acids (Street et al. 2008, FIG. 2). The hydrophobic region at its C-terminus is essential for AHL1's nuclear localization (Fujimoto et al., 2004). However, no other biological function for this domain is known. The PPC domain exists as a single domain in proteins from Bacteria and Archaea. Whereas in plant species like Arabidopsis, it is intimately associated with the AT-hook motif (Fujimoto et al., 2004). This high conservation through evolution and the large number of family members identified in Arabidopsis suggests that this domain is important for plant development.

X-ray crystallography analysis of the thermophylic archea Pyrococcus horikoshii PPC domain at a 1.6 Å resolution reveals a trimer complex with the subunit-subunit contacting surface maintained by a hydrophobic region that is formed by several anti-parallel β-sheets (Lin et al., 2005; Lin et al., 2007). Secondary structure prediction of the SOB3 PPC domain suggests that it has the same arrangement of anti-parallel (β-sheets as in the P. horikoshii PPC protein. Herein, we conceived and have corroborated that Arabidopsis AHL proteins associate with each other in homo- or hetero-complexes and that the PPC domain is responsible for this interaction.

The AT-Hook Motif. The AT-hook motif has been shown to interact with A/T-rich stretches of DNA (Reeves and Nissen, 1990; Huth et al., 1997; Bewley et al., 1998). Three types of AT-hook motifs have been identified (Aravind and Landsman, 1998). The AHL proteins have the Type II AT-hook motif, with a central arginine-glycine-arginine (R-G-R) core element flanked by prolines (Street et al. 2008, FIG. 2). While the R-G-R core represents a concave surface and perfectly fits in the minor groove, the proline residues flanking this core region direct the rest of peptides out of the minor groove and provided millimolar-range binding affinity to DNA. The residues downstream of the R-G-R core provide additional affinity and specificity to DNA (Huth et al., 1997). The type II AT-hook motif in the AHL proteins has conserved sequences, glycine-serine-lysine-asparagine-lysine-x-lysine-x-proline, at carboxy end of the R-G-R core. This region is unique to the AHL protein family and has been suggested to provide extra DNA contact (Huth et al., 1997). During an EMS-induced sob3-D intragenic suppressor screen, a sob3-4 null allele and two missense alleles, sob3-5 and sob3-6 were identified to repress the suppression of hypocotyl growth (Street et al. 2008, FIG. 2). The sob3-6 mutation causes an R77>H conversion in the AT-hook motif whereas the sob3-5 lesion causes an adjacent G80>Q change demonstrating the importance of the AT-hook core and flanking conserved sequences for SOB3 function.

ESC has been shown to bind with A/T-rich DNA sequence in the promoter region of pea PRA gene (Lim et al., 2007). HRC, as well as AHL15, can bind the GNFEI (GA-negative feedback element I) of the gibberellins 3-oxidase (GA3ox) promoter, possibly as a means for regulating a GA-negative feedback loop (Matsushia et. al., 2007). AHL proteins in Catharanthus roseus have been found to bind the jasmonate-responsive element region in the promoter of ORCA3 (octadecanoid-derivative responsive Catharanthus AP2-domain) gene (Endt et al., 2007). In Specific Aim 3 we will examine the DNA binding properties of SOB3 and the sob3-5 and sob3-6 proteins with their mutated type II AT-hook motifs.

Over-expression of SOB3 and ESC represses hypocotyl growth in seedlings and induces robust plant growth in adults. Activation-tagging enhancer elements inserted upstream of the SOB3 promoter region generated the over-expressed dominant allele, sob3-D (FIG. 3; Street et al., 2008). Over-expression of SOB3 represses light-grown hypocotyl elongation in both phyB-4 mutant and wild-type seedlings (Street et al. 2008, FIGS. 3A and B). Over-expression of ESC confers similar seedling phenotypes (Street et al. 2008, FIGS. 3C and D). In contrast, for adults, over-expression of SOB3, ESC, HRC, AHL18 and AHL22 results in more robust adult plants with elongated primary stem growth and expanded leaves (FIG. 4; Jiang, 2004; Lim et al., 2007; Street et al., 2008; Xiao et al., 2009). Constitutive expression of AHL members also leads to delayed flowering and senescence (Lim et al., 2007; Street et al., 2008; Xiao et al., 2009). Long-day-grown sob3-D plants flowered approximately one week later than wild-type plants though the number of rosette leaves at flowering was similar for both genotypes. The same flowering phenotype is caused by over-expression of AHL22 and AHL18 (Xiao et al., 2009). ESC over-expression also delays flowering while enhancing photosynthesis in mature plants (Lim et al., 2007).

Analyzing the functions of AHL gene family based on gain-of-function studies hints at their roles in regulating various aspects of plant growth and development. Over-expression of AHL genes increases biomass via expanded leaf areas, enhanced primary stem growth and enlarged organ size together with enhanced photosynthesis capacity and delayed flowering and senescence. In fact, the HRC gene has been patented for increasing plant biomass (Jiang, 2004). However, focusing on these data can be misleading due to high levels and potential lack of specificity in gene expression. Therefore loss-of-function analysis must be coupled with over-expression studies in order to fully understand the genetic role of a given gene or (semi-) redundant gene family.

Loss-of-function analysis of SOB3 and ESC. The sob3-4 null allele (Q47>stop) was identified as an EMS-induced intragenic suppressor of the sob3-D short hypocotyl phenotype. The esc-8 null allele (Q43>stop) was obtained from the Seattle TILLING project (Till et al., 2003). Singe nulls are phenotypically wild-type as seedlings and adults. In contrast, the sob3-4 esc-8 double mutant has a significantly longer hypocotyl than the wild type in multiple fluence rates and wavelengths of light (FIG. 5; Street et al., 2008). This loss-of-function analysis unequivocally demonstrates that SOB3 and ESC redundantly modulate light-mediating seedling development. Xiao et al. (2009) recently reported that RNAi-knockdown of SOB3 and AHL18 in an ESC- and AHL22-null background confers seedlings with longer hypocotyls than the wild type or ahl22 null, suggesting that all four AHL members may function redundantly to regulate hypocotyl growth.

Applicants have provided the most rigorous loss-of-function analysis for any members of the AHL gene family to date. Additional loss-of-function analysis will facilitate further understanding the biological roles of these DNA-binding proteins. According to certain embodiments, Applicants can generate and characterize higher order null alleles for AHL family members chosen based on previous studies and identified co-expression networks.

Two intragenic suppressor alleles, sob3-5 and sob3-6, confer dramatic long hypocotyl phenotypes. Double-null analysis of sob3-4 esc-8 demonstrates that SOB3 and ESC act redundantly to repress light-grown hypocotyl elongation. However, the relatively subtle long-hypocotyl phenotype suggests that other family members may also be involved in this process (Street et al. 2008). Two missense alleles, sob3-6 and sob3-5, confer much longer hypocotyls than the wild type or the sob3-4 esc-8 double null (Street et al. 2008, FIGS. 5 and 6). They both cause amino acid changes in and near the AT-hook motif respectively (Street et al. 2008, FIG. 2). The more severe allele of these two, sob3-6, is caused by a R77>H conversion in the first R of the R-G-R core region possibly abolishing the DNA binding capacity of this protein. The observation that this allele was originally identified as a heterozygous intragenic suppressor of sob3-D, coupled with a more severe phenotype than the sob3-4 esc-8 double mutant, suggests that the sob3-6 allele is acting as a dominant-negative mutation (Street et al. 2008). We present unpublished results that show the dominant-negative interpretation of the sob3-6 lesion.

### SUMMARY OF EXEMPLARY EMBODIMENTS

Particular preferred aspects provide an isolated nucleic acid encoding a polypeptide comprising SEQ ID NO:3, SEQ ID NO:6, a polypeptide having at least 93% or at least 95% sequence identity with SEQ ID NO:3, or a polypeptide having at least 75% or at least 80% sequence identity with SEQ ID NO:6. In certain aspects, the nucleic acid comprises SEQ ID NO:1, SEQ ID NO:2, SEQ ID NO:4 or SEQ ID NO:5.

Additional exemplarly aspects provide an isolated polypeptide comprising SEQ ID NO:3, SEQ ID NO:6, a polypeptide having at least 93% or at least 95% sequence identity with SEQ ID NO:3, or a polypeptide having at least 75% or at least 80% sequence identity with SEQ ID NO:6.

Further exemplary aspects provide a Camelina AHL polypeptide having a mutation of the AT hook domain that confers a dominant negative phenotype as disclosed herein. In certain embodiments, the polypeptide comprises a mutation in the AT hook domain of SEQ ID NO:3, SEQ ID NO:6, of a polypeptide having at least 93% or at least 95% sequence identity with SEQ ID NO:3, or of a polypeptide having at least 75% or at least 80% sequence identity with SEQ ID NO:6. In particular aspects, the polypeptide lacks the AT hook domain thereof. In certain embodiments, the mutant polypeptide yet comprises an intact or functional PPC domain.

Yet additional exemplary aspects provide a method of generating modified plants, comprising introducing into, or engineering in a plant cell, a nucleic acid encoding a mutant AHL protein having a mutation of the AT hook domain that confers a dominant negative phenotype as disclosed herein, provided that if the mutant AHL protein comprises an Arabadodpis thaliana (AT) Sob3 mutant, that the plant cell is not an AT plant cell. In certain method embodiments, the mutant AHL protein comprises a mutant Sob3 or Esc polypeptide. In certain method embodiments, introducing into, or engineering in comprises at least one of plant breeding and recombinant DNA and/or transformation methods. In certain method aspects, the mutant AHL protein is based on, or derived from a Camelina, or Arabadodpis thaliana (AT) AHL protein. In certain embodiments of the methods, the plant cell is of Brassica, Arabidopsis, soybean (Glycine max), canola (Brassica napus or B. rapa), sunflower (Helianthus annuus), Crambe (Crambe abysinnica); Black Mustard; Yellow Mustard (Sinapis alba); Oriental Mustard (Brassica juncea); Broccoli (Brassica oleracea italica); Rapeseed (Brassica napus); Meadowfoam (Limnanthes alba), Radish (Raphanus sativus); Wasabi (Wasabia japonica); Horseradish (Cochlearia Armoracia); Cauliflower; Garden cress (Lepidium sativum); Watercress (Nasturtium officinalis); and Papaya (Carica papaya), canola (rape), wheat (triticum), rice, corn, or a monocot. In certain embodiment, the dominant negative phenotype comprises taller seedlings.

Yet further aspects, provide a recombinant or genetically modified plant or plant cell comprising a nucleic acid encoding a mutant AHL polypeptide having a mutation of the AT hook domain that confers a dominant negative phenotype as disclosed herein, provided that if the mutant AHL protein is an Arabadodpis thaliana (AT) Sob3 mutant, the plant or plant cell is not an AT plant or plant cell. In certain aspects, the mutant AHL protein comprises a mutant Sob3 or Esc polypeptide. In particular embodiments, the mutant AHL protein is based on, or derived from a Camelina, or Arabadodpis thaliana (AT) AHL protein. In certain aspects, the phenotype of the plant comprises taller seedlings. In certain aspects, the plant is derived using a method according to any one of claims 8-13.

### BRIEF DESCRIPTION OF THE DRAWINGS

FIG. 1 shows sob3-6×sob3-D F1 generation plants partially suppress the sob3-D phenotype. (A) Hypocotyl length of F1 hybrids compared to sob3-D hypocotyls (B) Adult phenotype of sob3-6/sob3-D F1 hybrid, sob3-D, sob3-6 heterozygote and wild-type plants at 32 days after germination.

FIG. 2 shows over-expression of sob3-6 allele in wild-type Arabidopsis recapitulates the sob3-6 phenotype in seedlings and confers dwarfing in some adults. (A) Hypocotyl comparison of 5-day-old wild-type (Col-0), sob3-5, sob3-6 and six independent sob3-6 overexpression primary transformant lines. Scale bar=5 mm. (B)-(E) Various adult phenotypes observed in the sob3-6 primary transformant lines: similar to the wild type (B), semi-dwarf (C), dwarf (D) and severe dwarf (E).

FIG. 3 shows SOB3 and ESC interact with each other in Y2H assay. (A) SOB3 was used as prey(pACT2) and ESC was used as bait (pBTM116). Five individual colonies were picked and re-plated on SDII (selection medium containing leucine and histidine) or SDIV (selection medium without leucine and histidine). Pictures were taken 3 days after plating. (B) SOB3 was used as bait and ESC was used as prey.

FIG. 4 shows protein-protein interaction among SOB3, ESC and HRC proteins by BiFC. Onion epidermal cells were transformed with the indicated plasmid combination. A monomeric red fluorescent protein (pSAT6-mRFP) was used with each combination as a positive control. Each figure shows four channels observing the monomeric red fluorescent signal (I), yellow fluorescent signal (II), white field view (III) and overlapping view of I, II and III (IV).

FIG. 5 shows that a mutation in the AT-hook motif does not abolish the nucleus localization of AHL protein and protein-protein interaction. Onion epidermal cells were transformed with the indicated plasmid combination. A monomeric red fluorescent protein was also used with each pair for positive control. The esc-11 allele was created to harbor the same mutation as in sob3-6 allele.

FIG. 6A shows the phylogenic tree of AHL gene family taken from Street et al., 2008. AHL members that exist in each co-expression network are shown with symbols described in the legend table. AHL members that are not part of a co-expression network are marked with a cloud symbol. FIGS. 6B and 6C shows the co-expression pattern of the AHL gene family. (B) Co-expressed gene networks of AHL genes revealed by the ATTED-II database (Obayashi et al., 2007; Obayashi et al., 2009). Pairwise Pearson's correlation coefficients were indicated for each pair of co-expressed genes. (C) Thumbnail image of e-Northern results of indicated AHL genes generated by BAR (The Botany Array Resource) database (Toufighi et al., 2005). All publicly available micro-array data were used in this analysis.

FIG. 7 shows the effects of HERCULES (HRC1) overexpression in plants. Taken from Jiang 2004, U.S. Pat. No. 6,717,034, Method for modifying plant biomass.

FIG. 8 shows the hypocotyl length in cm of Camelina seedlings versus the days after planting. The graph shows the difference in hypocotyl length of Camelina seedlings between wildtype (non-transgenic; top panel) and Atsob3-6 overexpressing plants (transgenic; bottom panel).

FIG. 9 shows the hypocotyl length in cm of certain T2 generation Camelina seedlings.

FIG. 10 shows T3 generation Camelina seedlings over-expressing Atsob3-6 (right) compared to wild type syblings (left) after being planted on 1 cm of moist Palouse silt-loam and then covered with 8 cm of dry Palouse silt loam. Ten seedlings were placed in each pot. 30 to 50% of the transgenic seedlings emerged from this deep planting whereas no wild type plants did. After this experiment was completed, it was determined that both pots experienced 100% germination. Experiment has been repeated three times.

FIG. 11 shows that the weight of 100 T4 generation Camelina seeds over-expressing Atsob3-6 (right) is heavier when compared to a transgenic line expressing the empty-vector (left). The transformant line (right) also yields seedlings with longer hypocotyls than empty-vector control line.

FIG. 12 shows that the weight of 100 homozygous Arabidopsis sob3-6 mutant seeds (left) is heavier when compared to a wild-type control (right). Raw values are presented above the bars along with ±SEM.

FIG. 13 shows the weight of 100 T3 generation transgenic Arabidopsis seeds over-expressing Atsob3-6 compared to the wild type. Transformant-2 (far-right) is heavier when compared to the wild type (far-left) and Transformant-1 (center). Transformant-1 confers a hypocotyl phenotype that is the same as the wild type. Transformant-2 confers a longer hypocotyl than the wild-type. Raw values are presented above the bars along with ±SEM.

FIG. 14 shows that the esc-11 mutation also confers a long hypocotyl phenotype in Arabidopsis T1 transgenic seedlings. The esc-11 allele was created with the same mutation as sob3-6 using site-directed-mutagenesis. Wild-type (Col-0) were transformed with an empty vector control (far-left), the wild-type copy of ESC (ESCox1 and ESCox2) or with the esc-11 allele (esc-11ox1 to esc-11ox7). The ESCox and esc-11ox alleles were driven by the CaMV35S promoter. Scale bar=5 mm.

FIG. 15 shows that the overexpression of the SOB3 PPC domain and the linker region between the PPC domain and the AT-hook is sufficient to confer a long hypocotyl phenotype in T1 transgenic Arabidopsis seedlings. A wild-type (Col-0) seedling transformed with an empty vector control is shown on the left. A wild-type T1 seedling transformed with the linker region and the PPC domain driven by the CaMV35S promoter is shown on the right. Scale bar=2 mm.

### DETAILED DESCRIPTION

Overview. Applicants generally use Arabidopsis seedling development as a barometer for exploring changes in plant growth in response to both external cues and internal signaling pathways. For example, to complement traditional loss-of-function genetic approaches, gain-of-function gene-over-expression strategies can be used to identify components which may be involved in light-mediated seedling development (Weigel et al., 2000). This activation tagging approach allows for identification of genes that are small and/or part of a functionally redundant family and thus, not easily identifiable in loss-of-function mutant screens (Neff et al., 1999; Turk et al., 2005; Ward et al., 2005; Ward et al., 2006; Zhang et al., 2006; Street et al., 2008).

sob3-D (ACTIVATION-TAGGED SUPPRESSOR OF PHYTOCHROME B-4, #3-DOMINANT) was identified in a screen for extragenic suppressors of the long-hypocotyl phenotype conferred by a weak photoreceptor mutation, phytochrome B-4 (Street et al., 2008). SOB3's closest family member, ESCAROLA (ESC), was identified in an independent activation-tagging screen (Weigel et al., 2000). These two genes belong to the AT-HOOK MOTIF NUCLEAR LOCALIZED (AHL) family which is defined by containing one or more AT-hook DNA-binding motif(s) and a PLANT AND PROKARYOTE CONSERVED/DOMAIN OF UNKNOWN FUNCTION #296 (PPC/DUF296) (Fujimoto et al., 2004).

Over-expression of SOB3/AHL29 or ESC/AHL27 confers repressed hypocotyl elongation for seedlings grown in the light but not in darkness. As adults, these gene-over-expression plants develop larger organs including expanded leaves and enlarged flowers and fruits together with delayed flowering and senescence (Street et al., 2008). Over-expression of other AHL gene members also enhances adult leaf and stem growth (Jiang, 2004; Lim et al., 2007; Xiao et al., 2009). Single loss-of-function mutants for either SOB3 (sob3-4) or ESC (esc-8) have phenotypes similar to the wild type. In contrast, the sob3-4 esc-8 double mutant confers enhanced seedling hypocotyl growth under continuous white, red, far-red and blue light. Taken together, SOB3, ESC, and possibly other AHL genes, such as the next closest family member HERCULES (HRC/AHL25), function in a redundant manner to regulate hypocotyl elongation in response to light at the seedling stage and possibly flowering time and biomass for adult plants. However, the mechanism of action for AHL proteins has until now remained unknown.

The present disclosure furthers knowledge of how SOB3, ESC, HRC and other related AHL gene family members regulate growth in seedlings as well as adult plants. Apoplicants' data indicates that the redundant relationship shared by SOB3, ESC and likely HRC results from physical interactions with each other in vivo. Further examination of the physical interactions between these and other AHL proteins is a key step towards understanding the biochemical mechanism by which the AHL gene family regulates plant growth. Structure/function analysis allows investigation of the roles of two conserved domains, the AT-hook motif and the PPC/DUF296 (PPC) domain, in protein-DNA and protein-protein interaction. Gain-of-function and loss-of-function analysis of a subset of AHL gene family members, accompanied with the study of the dominant-negative sob3-6 allele, allowed us to examine the role of this suite of genes in regulating hypocotyl growth, flowering time, adult stature, photosynthesis, senescence and other aspects of plant development. These present studies transform and extend the understanding of the mechanism by which the AHL gene family regulates plant growth and development.

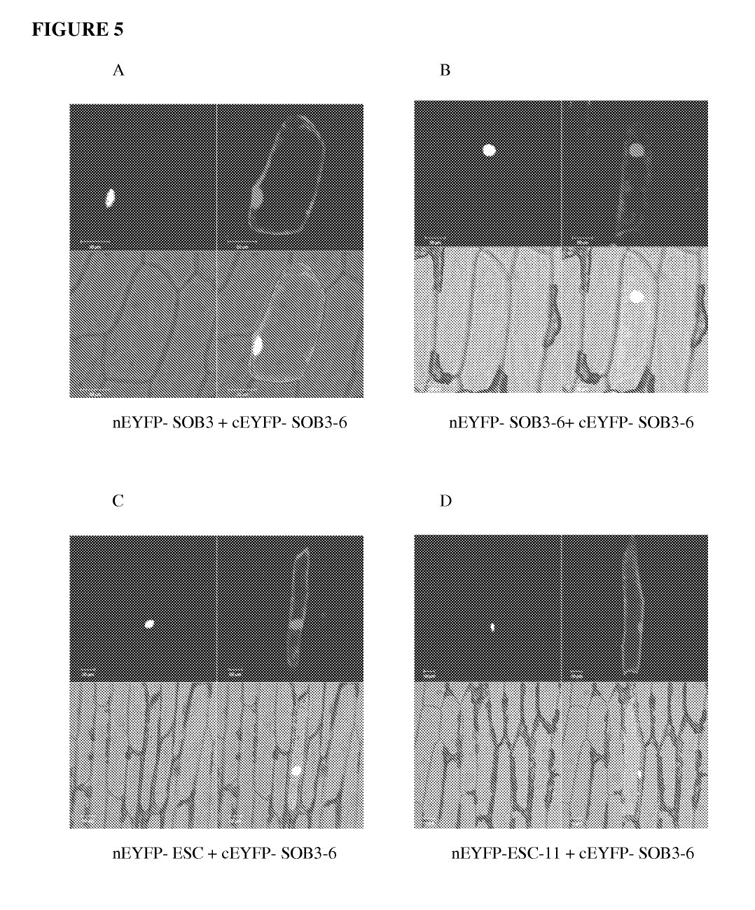

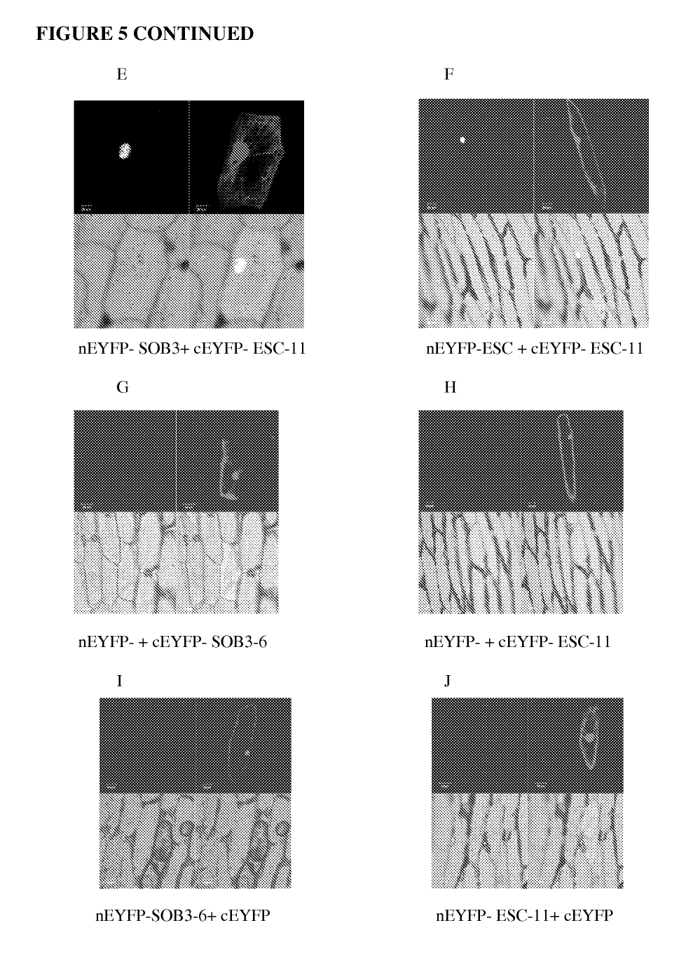

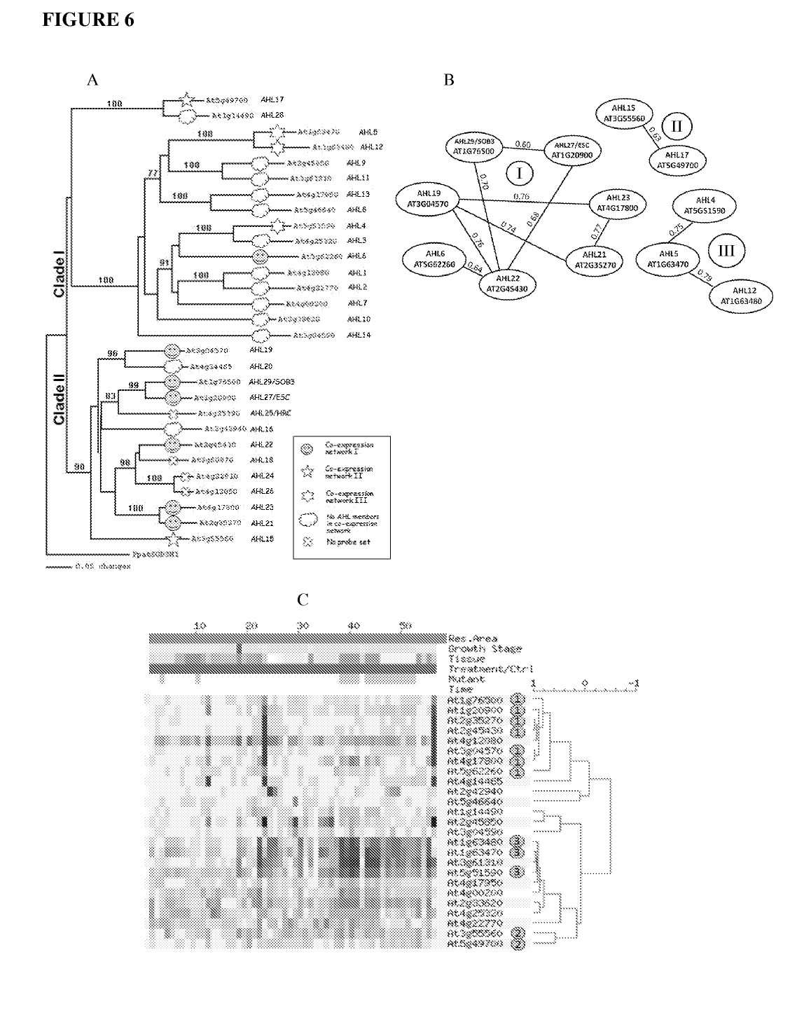

The dominant-negative nature of sob3-6. The two missense alleles of Arabadopsis, sob3-5 and sob3-6, are quite interesting given that they each have more severe long-hypocotyl phenotypes than the sob3-4 esc-8 double mutant (Street et al. 2008; FIGS. 5 and 6).

SOB3 Over-Expression Suppresses the Long-Hypocotyl Phenotype of phyB-4.

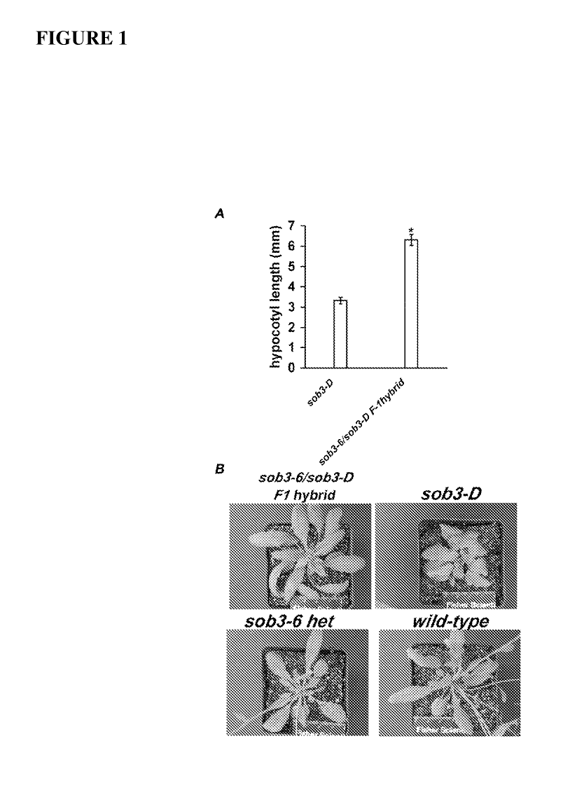

SOB3 was identified in a gain-of-function activation-tagging mutant screen for novel, dominant suppressors of the long-hypocotyl phenotype conferred by the weak phyB allele, phyB-4 (Ward et al., 2005; Weigel et al., 2000). sob3-D phyB-4 T2 plants segregated as a single-locus T-DNA insertion and flanking genomic DNA was cloned by Kpn I-fragment plasmid rescue. Sequencing of the rescued plasmid and BLASTn analysis revealed that the transgene enhancer elements were inserted on chromosome I, 497 by upstream of the annotated open-reading-frame (ORF) Atlg76500. No other predicted ORFs were found in the 4.5 kb insert of the rescued plasmid. The accumulation of Atlg76500 transcript was elevated in sob3-D phyB-4 plants compared to the wild-type (Street et al. 2008, FIG. 1B). Further, the over-expression seedling phenotypes were recapitulated by transforming phyB-4 plants with a transgene carrying a portion of the rescued plasmid containing the 35S enhancer elements, the Atlg76500 ORF, and flanking regions of genomic DNA (Street et al. 2008, FIG. 1). These results demonstrate that the sob3-D phenotype is caused by the over-expression of Atlg76500/SOB3 (FIG. 1).

Although sob3-D hypocotyls were shorter when grown in white light in both wild-type (Col-0) and phyB-4 backgrounds, the hypocotyls elongated normally in the dark, indicating that the sob3-D mutant does not cause a general growth defect but perturbs development in a light-dependent manner (Street et al. 2008, FIG. 1A). One possible explanation for the light dependency of the sob3-D seedling phenotype is that light regulates SOB3 expression. RT-PCR analysis of Atlg76500 from light- and dark-grown wild-type and phyB-4 seedlings, however, demonstrated similar levels of transcript accumulation, indicating that SOB3 is expressed in seedlings and is not light-regulated at the transcriptional level (Street et al. 2008, FIG. 1B). The lack of light regulation of SOB3 points to the alternative possibility that SOB3 over-expression impinges on light-signaling pathways.

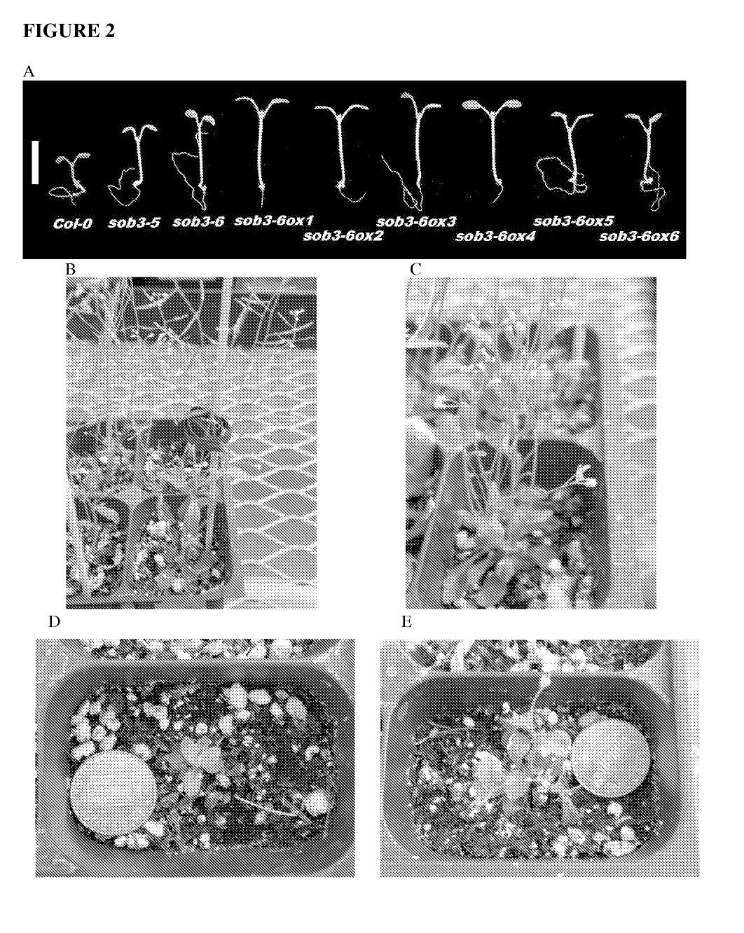

sob3-D Plants Exhibit Altered Cell Expansion Dynamics and Delayed Senescence
SOB3 over-expression also resulted in altered adult phenotypes. The first conspicuous adult sob3-D phenotype observed was the slower development of rosette structures relative to wild-type Col-0 plants. Fourteen-day-old long-day-grown (16 hrs light: 8 hrs dark) Col-0 plants were larger than sob3-D plants (Street et al. 2008, FIG. 2A). This trend continued until 28 days after germination, when sob3-D leaves became larger than Col-0 (Street et al. 2008, FIG. 2A). Though long-day-grown sob3-D plants flowered approximately one week later than wild-type plants, the number of rosette leaves at flowering was similar for both genotypes (Street et al. 2008, Supplemental FIG. 1). sob3-D phyB-4 plants showed a similar growth pattern as sob3-D plants (Street et al. 2008, Supplemental FIG. 2). sob3-D and sob3-D phyB-4 plants also senesced later than the wild-type. After 44 days of growth, phyB-4 and the wild-type began senescing, whereas sob3-D plants were still green and actively growing (Street et al. 2008, FIG. 2A, Supplemental FIG. 2). Eventually, sob3-D plants developed larger leaves and flowers than the wild-type (Street et al. 2008, FIG. 2B, C). The sob3-D mutation conferred similar phenotypes in a wild-type background (Street et al. 2008, FIG. 2).

The increased organ size caused by the sob3-D allele could result from increased cell proliferation, cell expansion, or a combination of both processes. To investigate the cause of increased organ size, epidermal imprints of 16, 23 and 30-day-old 4^{th} leaves of Col-0 and sob3-D were made and cell area determined. As shown in FIG. 2D (Street et al. 2008), sob3-D leaf epidermal cells were significantly smaller than Col-0 at 16 days. At 23 and 30 days, however, sob3-D epidermal cells were significantly larger than the wild-type (Street et al. 2008, FIG. 2D). The increased leaf size can, therefore, be attributed to cell expansion, not cell proliferation. Taken together, SOB3 over-expression leads to a delay in cell expansion in the light, explaining the slower growth phenotypes exhibited in both seedlings and adult plants. SOB3 over-expression eventually leads to excessive cell expansion, leading to the over-growth phenotypes seen in sob3-D adult plants.

### SOB3 is a Member of a Plant-Specific Protein Family

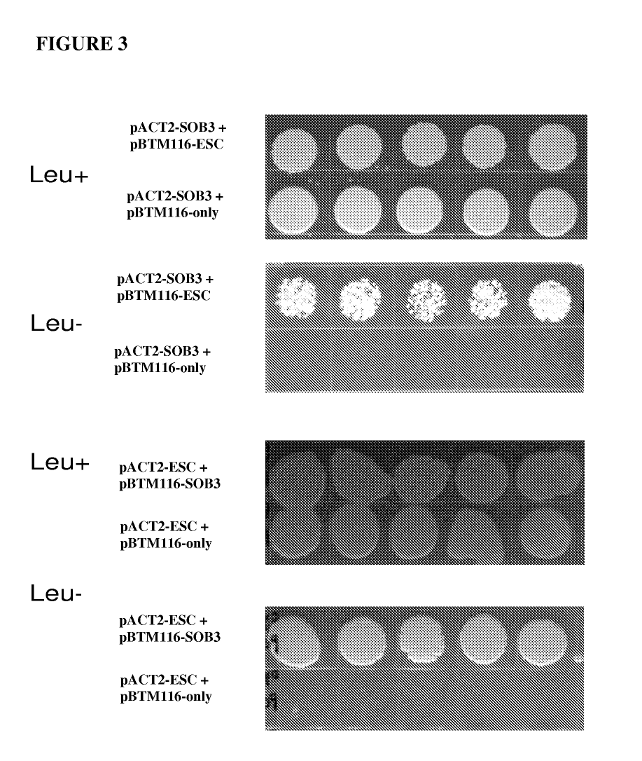

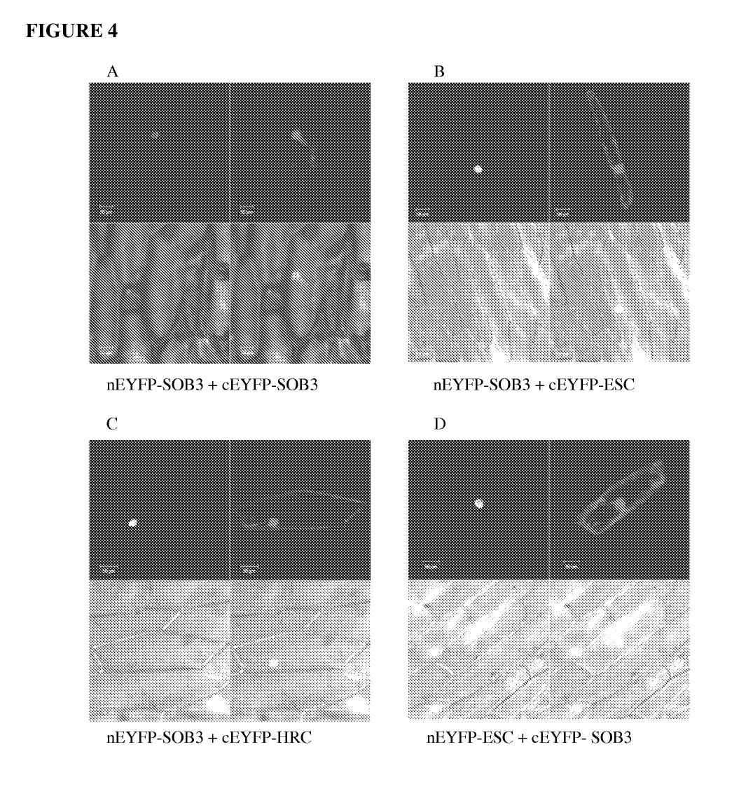

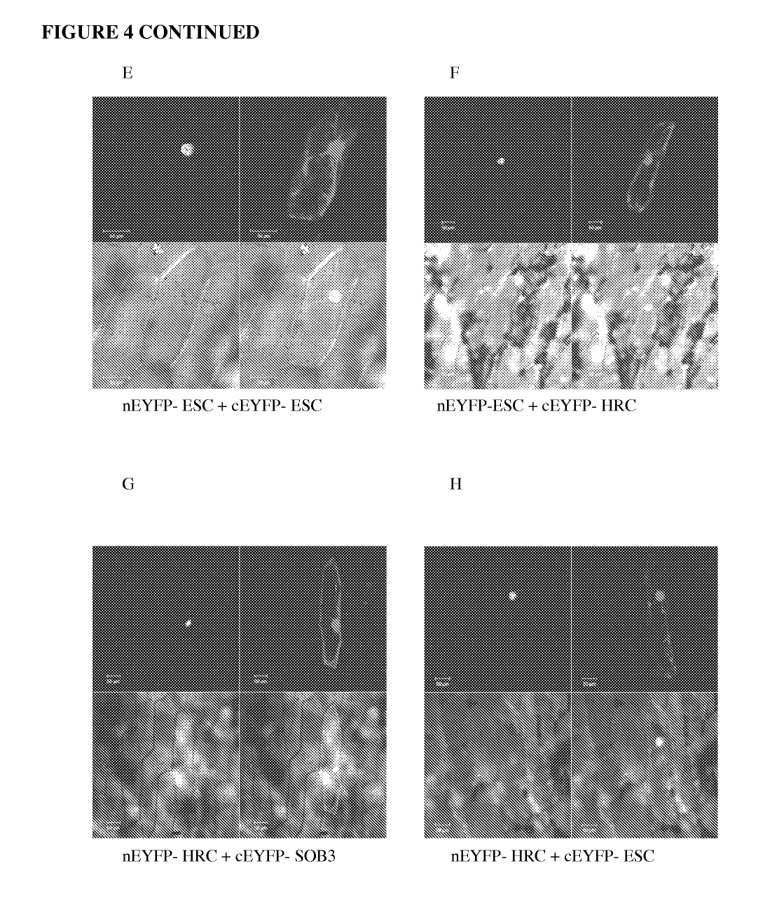

SOB3 encodes a protein containing a single AT-hook DNA-binding motif and a PPC (Plant and Prokaryotic Conserved) domain of unknown function (Fujimoto et al., 2004). A BLASTn analysis found one significant match in the Arabidopsis genome, Atlg20900/ESC (Weigel et al., 2000). There is synteny of several adjacent genes in the SOB3 and ESC chromosomal regions, suggesting that they may have arisen from a gene duplication event (Street et al. 2008, Supplemental FIG. 3). In addition, the ESC protein contains 74% (224/302) identical and 89% (270/302) similar amino acids when compared to SOB3 (Street et al. 2008, FIG. 3A). SOB3 and ESC belong to a gene family in Arabidopsis designated AHL (AT-hook motif nuclear localized protein) with SOB3 and ESC being AHL29 and AHL27, respectively (Fujimoto et al., 2004). SOB3/AHL29 and ESC/AHL27 have identical AT-hook motifs and a highly conserved PPC domain, suggesting that these two proteins may have similar function (Street et al. 2008, FIG. 3A). A BLASTp search revealed 28 annotated proteins similar to SOB3/AHL29 in Arabidopsis and homologs in plants with sequence data available (Street et al. 2008, Supplemental FIG. 4; Fujimoto et al., 2004). No SOB3/AHL29-like proteins containing both the AT-hook DNA-binding motif and the PPC domain were found in prokaryotes, fungi or animals, suggesting that SOB3/AHL29 is part of a conserved, plant-specific family of proteins (Fujimoto et al., 2004).
ESC Over-Expression Results in Phenotypes Similar to those Exhibited by sob3-D phyB-4 Plants
To determine whether ESC is similar to SOB3 in its gain-of-function state, transgenic plants over-expressing the ESC ORF (esc-OX) in the phyB-4 background were generated using a 5,044 by fragment of the rescued plasmid from the esc-1D activation tagged mutant (Weigel et al., 2000). Multiple independent T2 transgenic lines with increased levels of ESC transcript accumulation conferred short hypocotyls when compared to phyB-4 (Street et al. 2008, FIG. 3B, 3C). esc-OX phyB-4 plants also had adult phenotypes similar to those displayed by sob3-D phyB-4 plants (Weigel et al., 2000). These data suggest that SOB3 and ESC can play similar roles in plant development.

### SOB3 and ESC Proteins Accumulate in Hypocotyls

SOB3 and ESC transcripts were detected in seedlings (Street et al. 2008, FIG. 1B, 3C, 3D) but not adult leaves (Street et al. 2008, FIG. 3D) using a RT-PCR assay. The Genevestigator public microarray resources indicated that SOB3 and ESC are expressed in seedlings as well as in root tissue and developing siliques (Zimmerman et al. 2004). To further explore the tissue-specific expression pattern of SOB3 and ESC in seedlings, transgenic plants harboring a reporter gene β-glucuronidase (GUS) translational fusion under control of the SOB3 or ESC native promoter were constructed (SOB3:SOB3-GUS and ESC:ESC-GUS). Multiple homozygous single-locus-insertion lines were analyzed. Homozygous lines had shorter hypocotyls compared to wild-type control plants under dim white light conditions, suggesting that the GUS-fusion transgenes are functional (Street et al. 2008, Supplemental FIG. 5).

GUS activity expressed from both SOB3:SOB3-GUS and ESC:ESC-GUS transgenes was observed primarily in the vascular systems of seedlings, including the hypocotyls, cotyledons and roots in both dark- and light-grown plants (Street et al. 2008, FIG. 4A-D). In many lines, GUS activity was observed throughout the width of the hypocotyl (Street et al. 2008, FIG. 4A-D). GUS activity was also observed in the tips of cotyledons. In root tissues, GUS activity was observed in the root vasculature as well as the budding lateral roots (Street et al. 2008, FIG. 4E, F). SOB3:SOB3-GUS and ESC:ESC-GUS lines were identical in their overall staining patterns in seedling tissues. These results support the hypothesis that SOB3 and ESC act in similar tissues during seedling development.

### SOB3 and ESC Localize to the Nucleus

To determine the sub-cellular localization of SOB3 and ESC, transgenic lines expressing a YFP-SOB3 or YFP-ESC translational fusion driven by the 35S promoter were constructed and live root tissue observed under a UV light. Plants transformed with these constructs displayed the short-hypocotyl phenotype typical of sob3-D plants, indicating that the fusion protein is functional. As shown in FIG. 4G-H, the YFP-SOB3 signal was detected in the nucleus of root-hair cells as confirmed by Hoechst nuclear counterstain. Similar results were obtained for a 35S:YFP:ESC fusion construct (Street et al. 2008, Supplemental FIG. 6). These protein localization results are consistent with the hypothesis initially suggested by the presence of the AT-hook domain that SOB3 and ESC are nuclear proteins that likely interact with DNA.

### Identification of SOB3 and ESC Loss-of-Function Alleles

Although gain-of-function/over-expression phenotypes and protein expression patterns provided clues as to SOB3 and ESC function, such as a possible role in light-dependent seedling development and a negative role in cell expansion processes, loss-of-function mutants were identified to further explore the role of these genes during seedling development. The SigNAL T-DNA insertion library contains three independent transgenic lines in which a T-DNA is inserted in the SOB3 promoter region (Alonso et al., 2003). Homozygous sob3-1, sob3-2, and sob3-3 plants carried T-DNAs inserted 4 bp, 3 bp and 50 bp upstream of the annotated start codon, respectively (Street et al. 2008, FIG. 5A). No obvious morphological seedling or adult phenotype was observed in any of these sob3 T-DNA alleles (data not shown). The detection of SOB3 transcript in all of these T-DNA insertion mutants leaves open the possibility that they may not be null alleles (Street et al. 2008, FIG. 5B).

Since none of the T-DNA insertion mutations could be confirmed as null alleles, an ethyl methanesulfonate (EMS) suppressor screen of sob3-D phyB-4 was undertaken to isolate loss-of-function alleles within the SOB3 ORF. M2-generation EMS-mutagenized pools of sob3-D phyB-4 plants were screened for the recovery of the phyB-4 long-hypocotyl phenotype. In a screen of approximately 100,000 M2 seedlings derived from 2500 M1 plants, three putative alleles within the SOB3 ORF were identified: sob3-4, sob3-5, and sob3-6 (Street et al. 2008, FIG. 5A, C). The sob3-4 allele caused a glutamine to a stop codon (Q47>stop) change before the two conserved domains in SOB3 (Street et al. 2008, FIG. 5A, C). In contrast, the sob3-5 and sob3-6 missense alleles caused amino acid changes near and within the putative AT-hook DNA-binding domain, respectively (Street et al. 2008, FIG. 5A, C). The sob3-5 allele caused a glycine to glutamine (G80>Q) change just outside the DNA-binding domain, whereas the sob3-6 allele caused an arginine to histidine (R77>H) change in the central amino acid of the AT-hook DNA-binding domain. The positions of the amino acid changes caused by the sob3-5 and sob3-6 alleles suggest that the AT-hook domain plays an important role in SOB3 function. Of the three new alleles generated in this intragenic suppressor screen, the sob3-4 nonsense allele was the best candidate for a null mutation based on gene structure and was chosen for further genetic characterization. As shown in Table I (Street et al. 2008), the sob3-4 mutation segregated in a Mendelian fashion in F2 populations generated from self-pollination of heterozygous SOB3/sob3-4 (sob3-D) parents (Table I, Street et al. 2008).

Nine mutant alleles of ESC were obtained from the Seattle TILLING project (Till et al., 2003). The esc-8 allele was chosen for further characterization as it contained a nonsense mutation (Q43>stop) before any of the conserved domains and was therefore likely to be a null allele (FIG. 5D, Street et al. 2008). The esc-8 allele also segregated in a Mendelian fashion (Table I, Street et al. 2008).

### Analysis of SOB3 and ESC Loss-of-Function Phenotypes

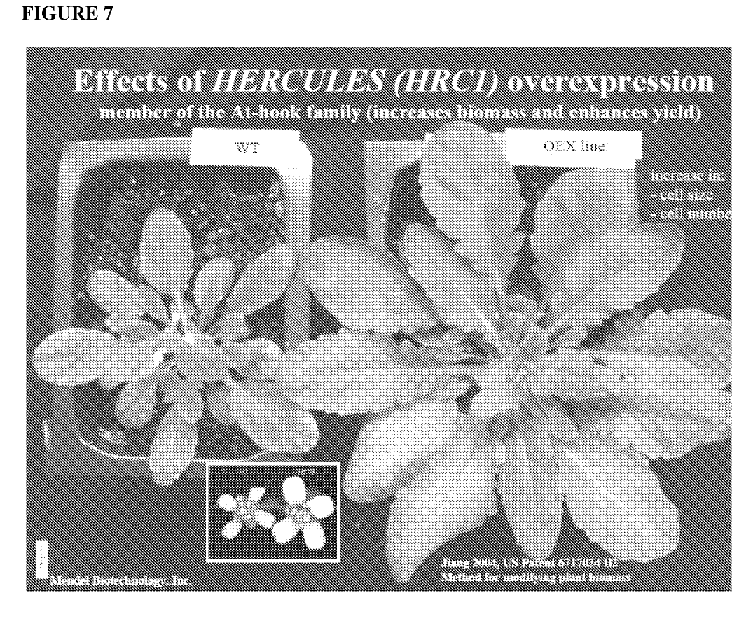

An F2 population segregating both alleles was used to generate the sob3-4 esc-8 double mutant, as well as wild-type, sob3-4, and esc-8 homozygotes as controls. None of the single or double mutants had a significant morphological phenotype in adult plants. For example, both sob3-4 and esc-8 single mutants, as well as the sob3-4 esc-8 double mutant, flowered at the same time as individuals in the wild-type sibling line (Supplemental FIG. 7, Street et al. 2008).

The loss-of-function mutants, however, did exhibit a light-dependent hypocotyl length phenotype at the seedling stage, further supporting the hypothesis that SOB3 and ESC play a role in seedling development. Since sob3-D and esc-OX gain-of-function mutations conferred shorter hypocotyls in the light (FIG. 1A, 3B, Street et al. 2008), the loss-of-function lines were used to perform fluence-rate-response assays (FRRAs) to test the hypothesis that the loss-of-function single mutants and the sob3-4 esc-8 double mutant would have the opposite phenotype. Under low-fluence-rates of white light, sob3-4 esc-8 seedlings had longer hypocotyls when compared to the wild-type or either single mutant (FIG. 6A, Street et al. 2008). A second double mutant using the sob3-2 T-DNA allele, sob3-2 esc-8, also exhibited a long-hypocotyl phenotype when grown in dim white light (FIG. 6B, Street et al. 2008). These results suggest that SOB3 and ESC are functionally redundant negative modulators of hypocotyl elongation, acting in one or more light-signaling pathways.

Single mutants carrying the sob3-6 missense allele also had a long-hypocotyl phenotype in the light compared to wild-type plants (FIG. 6C, Street et al. 2008), although sob3-4 nonsense mutants did not. The phenotype of the sob3-6 allele, which contains both the 35S enhancer and a missense mutation, suggests that over-expression of a protein with a mutated AT-hook domain confers a dominant-negative phenotype. Consistent with this hypothesis, a F1-generation cross between sob3-6 and sob3-D parents generated F1 hybrids with less severe sob3-D seedling and adult phenotypes, suggesting that the sob3-6 mutation suppresses the sob3-D allele (FIG. 1).

Interaction of Photoreceptor Mutations with sob3 and esc Loss-of-Function Mutations

To determine whether a particular photoreceptor pathway is involved in the altered de-etiolation response of sob3-4 esc-8 double mutants, FRRAs were carried out in continuous far-red, red and blue light. A significant difference in the double mutant relative to the wild-type and single mutants was observed in all three monochromatic light conditions under all fluence rates tested (Street et al. 2008, FIG. 6D, 6E, 6F). Furthermore, RT-PCR analysis showed no differences for accumulation of PHYA, PHYB, and CRY1 transcripts in the sob3-4 esc-8 and wild-type genetic backgrounds, suggesting that expression of these major photoreceptors are not altered (data not shown).

Triple mutants were generated containing sob3-4, esc-8 and null alleles of the far-red (phyA-211), red (phyB-9) and blue (cry-103) photoreceptors. These mutants were grown in far-red, red, and blue light conditions in which the sob3-4 esc-8 double mutant conferred a long-hypocotyl phenotype. In far-red light, hypocotyls of the sob3-4 esc-8 phyA-211 triple mutant were not significantly longer than hypocotyls of phyA-211 siblings (Street et al. 2008, FIG. 7A). This epistatic relationship suggests that SOB3 and ESC function downstream in the PHYA-far-red light pathway since PHYA is required to see the effect of sob3 and esc loss-of-mediated function mutations on hypocotyl elongation. In contrast, both the sob3-4 esc-8 phyB-9 and sob3-4 esc-8 cry-103 triple mutants had significantly longer hypocotyls than the single mutant photoreceptor lines (Street et al. 2008, FIG. 7B, 7C). These additive effects support the interpretation that the SOB3 and ESC activity is not limited to the PHYB- or CRY-mediated light signaling pathways.

SOB3 and ESC are involved in seedling development. Both SOB3 and ESC were identified through activation tagging mutagenesis and have similar gain-of-function phenotypes (FIG. 1; Weigel et al., 2000). The sob3-D and esc-OX adult phenotypes include slower development, delayed senescence and eventually larger organs with larger cell size, suggesting a role for SOB3 and ESC in cell expansion or differentiation (Street et al. 2008, FIGS. 1 and 2). The light-specific short-hypocotyl phenotype in these gain-of-function mutants suggests that SOB3 and ESC are involved in light-mediated seedling development (Street et al. 2008, FIGS. 1 and 3). It is possible to reconcile the seedling short hypocotyl phenotype with the adult large organs if sob3-D plants are slower growing than the wild-type in the light.

Furthermore, the similar gain-of-function phenotypes and the high DNA and protein sequence similarity between SOB3 and ESC suggest that these two genes are functionally redundant. The high degree of synteny around these two loci suggests that these genes are paralogs that have arisen via gene duplication (Street et al. 2008, Supplemental FIG. 3). This hypothesis is further supported by the observation that SOB3 and ESC are encompassed by larger regions predicted to arise by a chromosomal duplication event (Arabidopsis Genome Initiative, 2000). Although gain-of-function analyses can provide clues to gene function, the results should also be supported with loss-of-function experiments.

Loss-of-function sob3-4 esc-8 double mutant seedlings were less sensitive to white and monochromatic red, far-red and blue light, demonstrating that SOB3 and ESC can act redundantly. Phytochromes A and B are the primary far-red and red photoreceptors involved in hypocotyl responsiveness to light, respectively, whereas cryptochromes mediate blue light response (for review see: Franklin et al., 2005; Liscum et al., 2003; Neff et al., 2000). The observed sob3-4 esc-8 mutant phenotype suggests that SOB3 and ESC are negative modulators of seedling hypocotyl elongation and act as downstream integrators of light signaling. Further supporting this hypothesis is the phenotype of the sob3-4 esc-8 phyA-211 triple mutants compared to the phyA-211 single mutant (Street et al. 2008, FIG. 7A). This result suggests that PHYA is necessary to observe the sob3-4 esc-8 double mutant phenotype. Alternatively, since PHYA is the only far-red light receptor, it is also possible that light is required to see the effect of SOB3 and ESC loss-of-function. Native-promoter translational-GUS-fusion staining patterns were similar in light and dark grown seedlings (Street et al. 2008, FIG. 5). Since the protein distribution is similar in the light and dark, it is possible that SOB3/AHL29 and ESC/AHL27 protein activity is different in the light and dark. Taken together, these data support the hypothesis that SOB3 and ESC are downstream modulators of light-mediated hypocotyl responses.

Genetic and biochemical studies have revealed a complex network of individual interacting components necessary for a plant to properly interpret its light environment (for review see: Franklin et al., 2005; Moller et al., 2002; Neff et al., 2000). Phytochromes and cryptochromes have been shown to have partially redundant roles in seedling development (Lin et al., 1998; Neff and Chory, 1998; Ohgishi et al., 2004). The first downstream component identified, HY5, encodes a bZIP transcription factor that also has a long hypocotyl in multiple qualities of light, as well as other organ-development phenotypes, and may be an example of a downstream integrator of light and hormone responses (Cluis et al., 2004; Koornneef et al., 1980; Oyama et al., 1997). A HY5 homolog, HYH, was found to have some overlapping functions with HY5, particularly in blue light (Holm et al., 2002). SOB3 and ESC are similar in that they act partially redundantly in seedling development.

SOB3 and ESC are part of a conserved, plant-specific gene family. SOB3 and ESC are members of a family of genes that encode proteins containing an AT-hook motif (Fujimoto et al. 2004). AT-hook motifs are conserved in eukaryotes and some bacteria and are found in a wide variety of proteins involved in nuclear functions (Aravind and Landsman, 1998). The best characterized of this group are the High Mobility Group A (HMGA) proteins. HMGA proteins, which contain multiple AT-hook domains and are associated with cell proliferation or differentiation, are architectural transcription factors that recognize AT-rich stretches of DNA, (for review see: Grasser, 2003; Klosterman and Hadwiger, 2002; Reeves, 2001).

The Rice HMGA protein, PF1, is able to bind and enhance the activity of the Rice PHYA promoter suggesting a gene regulatory role for AT-hook proteins in photomorphogenesis (Martinez-Garcia and Quail, 1999). Single AT-hook domain containing proteins such as SOB3 and ESC are hypothesized to bind DNA and associate with the nuclear matrix (Fujimoto et al., 2004; Morisawa et al., 2000). A SOB3/ESC family member, AHL1, is suggested to encode a nuclear localized matrix attachment region (MAR) protein (Fujimoto et al., 2004). MARs are AT-rich sequences that attach chromosomal loops to the protein nuclear matrix and may play a role in transcriptional regulation (Paul and Ferl, 1998; Rudd et al., 2004). Recent work with other members of the SOB3/ESC gene family suggest that they are able to bind specific gene promoters involved in hormone responses (Matsushita et al., 2007; Vom Endt et al., 2007). These observations suggest that SOB3 and ESC act through DNA binding of AT-rich regions and act as accessory transcription factors.

SOB3 and ESC affect cell expansion. The opposite hypocotyl phenotypes of light-grown sob3-4 esc-8 and sob3-D mutants are most likely due to differential cell expansion, as hypocotyl growth in Arabidopsis involves cell elongation, not division (Gendreau et al., 1997). The capability of cells to expand in sob3-D mutants is not impaired as they elongate normally in the dark (Street et al. 2008, FIG. 1A). This result suggests that there is a role for SOB3 and ESC as negative regulators of hypocotyl elongation in the light.

The sob3-D and esc-OX enlarged adult organ size phenotype is also likely to be due to cell expansion, since epidermal cell size is increased in these over-expressing plants (Street et al. 2008, FIG. 2A). sob3-D and esc-OX plants take longer to develop compared to the wild-type and it is possible that this delay is due to an extended period of cell proliferation before cell differentiation and expansion. Leaves of sob3-D and esc-OX are twisted and not planar like a wild-type leaf, suggesting that the genetic program that determines wild-type leaf shape is disrupted in these plants. Genes such as the TCP (teosinte-branched, cycloidia, PCNA) family of transcription factors have been shown to be involved in this process by affecting cell proliferation and growth (Li et al., 2005; Nath et al., 2003; Palatnik et al., 2003).

Cell growth, division, expansion, endoreduplication, and differentiation are all factors involved in determining cell size, number and a plant's ultimate organ morphology (De Veylder et al., 2002; Grandjean et al., 2004; Li et al., 2005; Reddy and Meyerowitz, 2005; Sugimoto-Shirasu et al., 2005). It is an open question as to how all of these processes interrelate, though some progress has been made in identifying important components of cell state determinants and how this alters organ development. For example, loss-of-function of ANT plants have smaller aerial organs due to a lack of cell proliferation but have larger cells, due to a compensation mechanism (Mizukami and Fischer, 2000). ANT over-expression has the opposite phenotype, though unlike sob3-D and esc-OX plants, rosette leaf morphology is normal with wild-type cell size (Mizukami and Fischer, 2000). A gene hypothesized to act upstream of ANT, ARGOS, has a similar over-expression phenotype as ANT and is affected by auxin signaling (Hu et al., 2003). The closest paralog of SOB3/AHL29 and ESC/AHL27, HERCULES/AHL25 (HRC), also increases adult organ size when over-expressed (Jiang, 2004). SOB3, ESC and other gene family members can clearly affect adult organ morphology when over-expressed, suggesting an important role in plant architecture and a fundamental role in individual plant cells. The lack of an obvious adult phenotype in the sob3-4 esc-8 double mutant suggests that SOB3 and ESC may not play a role in adult development. Alternatively, other gene family members such as HRC may act redundantly with SOB3 and ESC in adult tissues.

Our studies facilitate determining the mechanisms by which SOB3 and ESC affect development. Without being bound by mechanism, it is possible that they act as transcription factors to regulate the expression of specific genes. The fact that SOB3 and ESC appear to act downstream of the photoreceptor network raises the possibility that they are part of a negative cell-expansion regulatory mechanism receiving input from the various signaling cascades of individual photoreceptors. Based on the GUS-fusion-expression data, SOB3/AHL29 and ESC/AHL27 are localized to the same tissues in seedlings in the light and the dark. Perhaps SOB3/AHL29 and ESC/AHL27 activity is mediated by post-translational modification in the light, or that SOB3/AHL29 and ESC/AHL27 proteins require the expression of genes specific to light-mediated development to affect hypocotyl elongation. Determining DNA binding sites and protein interacting partners as well as characterizing their loss-of-function phenotypes will shed more light on the roles the AHL gene family play in plant development.

### Comparison of Camelina Seedlings Over-Expressing Atsob3-6 (Right) Compared to Wild Type Syblings

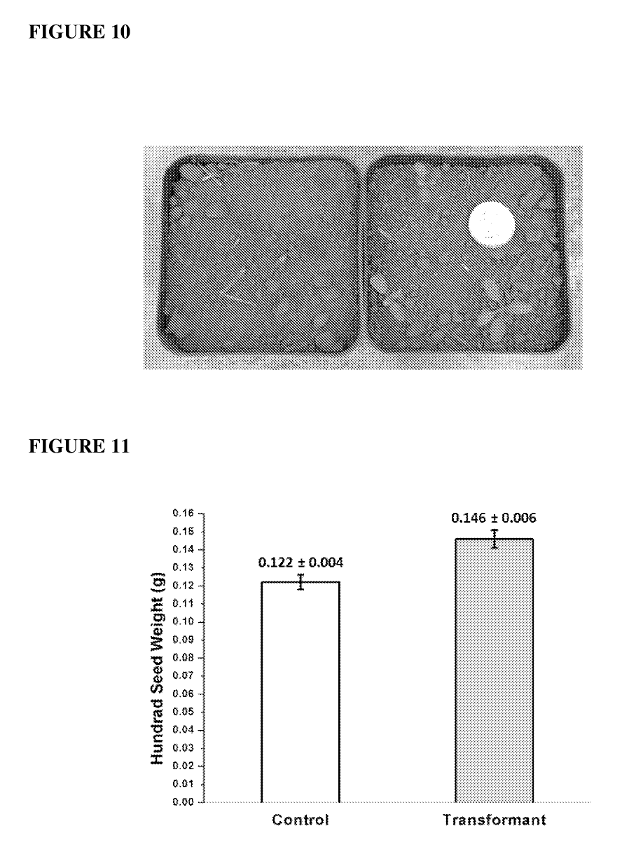

FIG. 10 shows T3 generation Camelina seedlings over-expressing Atsob3-6 (right) compared to wild type syblings (left) after being planted on 1 cm of moist Palouse silt-loam and then covered with 8 cm of dry Palouse silt loam. Ten seedlings were placed in each pot. 30 to 50% of the transgenic seedlings emerged from this deep planting whereas no wild type plants did. After this experiment was completed, it was determined that both pots experienced 100% germination. Experiment has been repeated three times.

FIG. 11 shows that the weight of 100 T4 generation Camelina seeds over-expressing Atsob3-6 (right) is heavier when compared to a transgenic line expressing the empty-vector (left). The transformant line (right) also yields seedlings with longer hypocotyls than empty-vector control line.

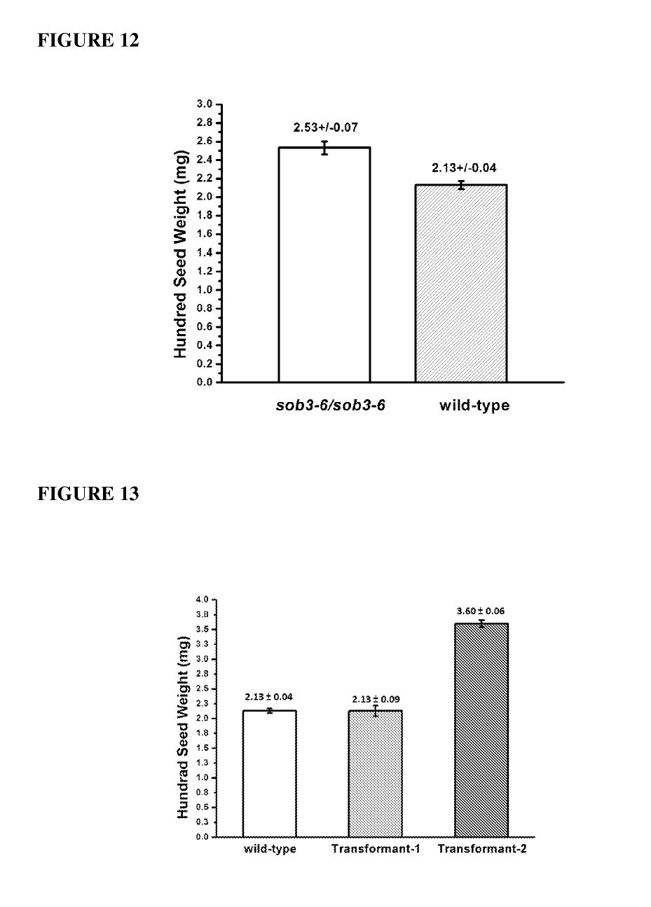

FIG. 12 shows that the weight of 100 homozygous Arabidopsis sob3-6 mutant seeds (left) is heavier when compared to a wild-type control (right). Raw values are presented above the bars along with ±SEM.

FIG. 13 shows the weight of 100 T3 generation transgenic Arabidopsis seeds over-expressing Atsob3-6 compared to the wild type. Transformant-2 (far-right) is heavier when compared to the wild type (far-left) and Transformant-1 (center). Transformant-1 confers a hypocotyl phenotype that is the same as the wild type. Transformant-2 confers a longer hypocotyl than the wild-type. Raw values are presented above the bars along with ±SEM.

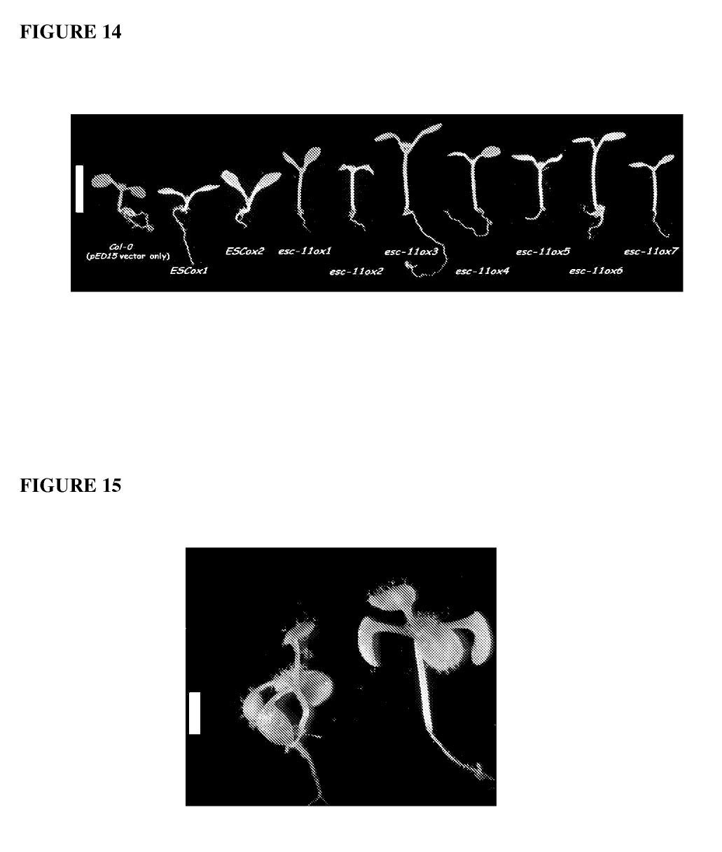

FIG. 14 shows that the esc-11 mutation also confers a long hypocotyl phenotype in Arabidopsis T1 transgenic seedlings. The esc-11 allele was created with the same mutation as sob3-6 using site-directed-mutagenesis. Wild-type (Col-0) were transformed with an empty vector control (far-left), the wild-type copy of ESC (ESCox1 and ESCox2) or with the esc-11 allele (esc-11ox1 to esc-11ox7). The ESCox and esc-11ox alleles were driven by the CaMV35S promoter. Scale bar=5 mm.

FIG. 15 shows that the overexpression of the SOB3 PPC domain and the linker region between the PPC domain and the AT-hook is sufficient to confer a long hypocotyl phenotype in T1 transgenic Arabidopsis seedlings. A wild-type (Col-0) seedling transformed with an empty vector control is shown on the left. A wild-type T1 seedling transformed with the linker region and the PPC domain driven by the CaMV35S promoter is shown on the right. Scale bar=2 mm.

In the following Examples 1-19, Applicants have, inter alia, cloned novel Camelina derived AHL genes and gene products for modulation of cell growth in plants. Particular aspects provide for manipulation of the AT-hook domain in Camelina AHL genes, including manipulation of the AT-hook domain (e.g., AT-hook domain mutants and modifications including but not limited to nonsense, missence, deletions, substitutions, muteins, fusions, etc.) in novel sequences SEQ ID NOS:1-6, which have substantial utility for modulation of cell growth in plants. Additional aspects provide modified plants comprising Camelina derived AHL genes and gene products, and modified versions thereof.

### Example 1

### Multiple T1 Transgenic Events Expressing CaMV35S:sob3-6 Recapitulated the Elongated Phenotype of the Backcrossed sob3-6 allele, some of which are Shown to be Even More Severe than the Original sob3-6 Lesion

Applicants' initial focus was on sob3-6 since this lesion is in the absolutely conserved AT-hook core and the seedling phenotype is more severe than sob3-5. We backcrossed this mutant with the wild type two times. In each backcross, the sob3-6 long-hypocotyl phenotype behaves as a single-locus dominant/semi-dominant trait that is 100% linked to the adjacent activation-tagging T-DNA. We have also over-expressed, in wild-type plants using Agrobacterium strain GV3101 and the floral dipping transformation method (Clough and Bent, 1998), the sob3-6 cDNA driven by the constitutive cauliflower mosaic virus 35S (CaMV 35S) promoter. Multiple T1 transgenic events expressing CaMV35S:sob3-6 have recapitulated the elongated phenotype of the backcrossed sob3-6 allele, some of which are even more severe than the original sob3-6 lesion (FIG. 2A). The dominant nature of this allele and the fact that the resulting phenotype is more severe than the long-hypocotyl conferred by the sob3-4 esc-8 loss-of-function mutant strongly supports the hypothesis that this is indeed a dominant-negative allele caused by a disruption of a conserved amino acid in the AT-hook core.

According to particular aspects of the present invention, the nature of the sob3-6 allele coupled with the X-ray crystallography analysis of the P. horikoshii PPC domain at suggests a model where SOB3 interacts with itself, perhaps via the PPC domain, and that each interacting partner requires a functional AT-hook core to properly bind DNA. Given the relatively strong phenotype of the original sob3-6 allele and the even more severe phenotypes in some CaMV35S:sob3-6 recapitulation lines (FIG. 2A-E), we conceived that SOB3 also interacts with other AHL family members such as ESC or HRC and that these hetero-interaction complexes are being titrated out by the sob3-6 mutant protein. Alternatively SOB3 and other AHL members could share similar non-AHL interacting partners that are being titrated away by the sob3-6 mutant protein. In either case, the extreme dwarf phenotypes found in some CaMV35S:sob3-6 expressing lines suggest that the AHL family plays an important role in seedling and adult plant development.

### Example 2

### SOB3 was shown to Associate with ESC

Protein-protein interaction studies. We first tested the hypothesis that SOB3 can associate with ESC using a yeast two-hybrid (Y2H) approach. For the Y2H assay, a lexA-based system was used consisting of pBTM116-D9 as a bait plasmid and pACT2 (Clontech, Palo Alto, Calif.) as a prey plasmid, together with the yeast reporter strain L40ccU3. Coding sequences of SOB3 and ESC proteins were recombined into both the bait and prey vectors via Gateway® reactions (Invitrogen, Carlsbad, Calif.). Our preliminary Y2H results suggest that SOB3 can associate with ESC (FIG. 3).

### Example 3

### SOB3, ESC and HRC were shown to Localize to the Nucleus and Physically Interact with Themselves and Each Other In Vivo

We further examined these interactions in planta using a transient Bimolecular Fluorescence Complementation (BiFC) Assay with onion epidermal cells biolistically transformed with Gateway® compatible vectors derived from pSAT4-DEST-n(1-174)EYFP-C1 and pSAT5-DEST-c(175-end)EYFP-C1(B) (Citovsky et al., 2006). The cDNAs for SOB3, ESC and HRC were cloned into each BiFC plasmid as in-frame translational fusions with either the N- or C-terminal half of a yellow fluorescent protein (YFP). Empty vectors were used as negative controls. Pairs of BiFC plasmids together with the pSAT6-mRFP plasmid encoding a red fluorescent protein (RFP) were co-bombarded into onion epidermal cells using a PDS-1000/He Biolistic transformation system (BIO-RAD). Reconstructed fluorescence was examined after 40 hours of incubation in the dark with a Zeiss LSM 510 META confocal microscope. The monomeric red fluorescence from the RFP was used to identify successful transformation into onion cells (FIG. 4). The fluorescence from reconstructed YFP observed in FIG. 4 A-I shows that SOB3, ESC and HRC localize to the nucleus and physically interact with themselves and each other in vivo. In the BIFC assay using negative controls (data not shown), yellow fluorescence could not be observed.

### Example 4

### sob3-6 and esc-11 Were Shown to also Associate with Each Other and with Themselves

Mutations in the AT-hook core motif do not abolish nuclear localization or protein-protein interaction. The AT-hook motif of AHL1 is essential for its A/T-rich DNA binding ability (Fujimoto et al., 2004). However, the AT-hook motif also contributes to the nuclear localization for high mobility group proteins (Sgarra et al., 2006; Cattaruzzi et al., 2007). Thus, it is possible that the sob3-6 protein may be disrupting its own activity and/or that of other family members by abolishing nuclear localization. We used the BiFC assay to examine if this mutation in the AT-hook motif affects the sob3-6 protein nuclear localization and its association with wild-type SOB3 and ESC. In addition, we used site-directed mutagenesis to generate an ESC cDNA with the same conserved mutation as in sob3-6: esc-11. BiFC analysis demonstrates that both the sob3-6 and esc-11 proteins can enter the nucleus and associate with wild-type SOB3 and ESC proteins. Furthermore, sob3-6 and esc-11 can also associate with each other and with themselves (FIG. 5). These results demonstrate that the sob3-6 and esc-11 mutations do not abolish nucleus localization or protein-protein interactions between AHL family members.

### Example 5

### Using PCR, Cloned cDNAs Similar to SOB3 and ESC were Obtained from Camelina, Demonstrating that the AHL Gene Family Exists in this Potential Oil-Seed Crop

Camelina ESC cDNA sequence 950 bp (SEQ ID NO: 1; coding
sequence):
TANAGCGGTGGACTTCTAGATCTTTCTAAACCTCTTCAGACCGGAGATTCACCACCA
GCACCTTCAACCGCAGGGTGGAATCAATCTTATTGACCAGCATCATCATCAGCATCA
GCAGCAACAACAACAACAACAACAGCMACCGTCGGATGATTCAAGAGAATCTGAA
CACTCAAACAAGGATCATCATCAACAGGGTCGACCCCGATTCAGACCCGAATACAT
CAAGCTCAACACCCGGGAAACGTCCACGTGGACGTCCGCCAGGATCTAAGAACAAA
GCAAAGCCACCGATCATAGTAACCCGTGACAGCCCCAACGCGCTTAGATCTCACGT
CCTTGAAGTATCTCCCGGAGCTGATATAGTTGAGAGTGTTTCCACTTACGCTAGGCG
GAGAGGGAGAGGCGTCTCCGTTTTAGGAGGGAACGGCACCGTTTCTAACGTCACTC
TCCGTCAGCCAGTCACTCCCGGAAACGGTGGTGGTGTGTCCGGAGGAGGAGGAGGA
GGAGTTGTGACTTTACATGGAAGATTTGAGATTCTTTCACTAACGGGGACTGTTTTG
CCACCTCCTGCGCCGCCTGGTGCAGGTGGTTTGTCAATATTTCTAGCCGGTGGGCAA
GGTCAGGTTGTTGGAGGAAGCGTGGTGGCTCCGCTTATTGCATCAGCTCCAGTTATA
CTAATGGCTGCTTCGTTCTCAAATGCGGTTTTCGAGAGACTACCAATGGAAGAGGA
AGAAGAAGAAGGTGCTGGTGCTGGCGGAGGGGGAGGAGGAGGACCACCGCAGATG
CAGCAAGCTCCCTCAGCATCGCCTCCGTCAGGCGTGACCGGTCAGGGACAGTTAGG
AGGTAATGTGGGTGGTTATGGGTTTTCTGGTGATCCTCATTTGCTTGGATGGGGAGC
TGGAACACCTTCAAGACCACTATTTTAATCGAANTTAAANTCCNGAATT
Camelina Esc ORF 780 bp (SEQ ID NO: 2):
ATGATTCAAGAGAATCTGAACACTCAAACAAGGATCATCATCAACAGGGTCGACCC
CGATTCAGACCCGAATACATCAAGCTCAACACCCGGGAAACGTCCACGTGGACGTC
CGCCAGGATCTAAGAACAAAGCAAAGCCACCGATCATAGTAACCCGTGACAGCCCC
AACGCGCTTAGATCTCACGTCCTTGAAGTATCTCCCGGAGCTGATATAGTTGAGAGT
GTTTCCACTTACGCTAGGCGGAGAGGGAGAGGCGTCTCCGTTTTAGGAGGGAACGG
CACCGTTTCTAACGTCACTCTCCGTCAGCCAGTCACTCCCGGAAACGGTGGTGGTGT
GTCCGGAGGAGGAGGAGGAGGAGTTGTGACTTTACATGGAAGATTTGAGATTCTTT
CACTAACGGGGACTGTTTTGCCACCTCCTGCGCCGCCTGGTGCAGGTGGTTTGTCAA
TATTTCTAGCCGGTGGGCAAGGTCAGGTTGTTGGAGGAAGCGTGGTGGCTCCGCTT
ATTGCATCAGCTCCAGTTATACTAATGGCTGCTTCGTTCTCAAATGCGGTTTTCGAG
AGACTACCAATGGAAGAGGAAGAAGAAGAAGGTGCTGGTGCTGGCGGAGGGGGAG
GAGGAGGACCACCGCAGATGCAGCAAGCTCCCTCAGCATCGCCTCCGTCAGGCGTG
ACCGGTCAGGGACAGTTAGGAGGTAATGTGGGTGGTTATGGGTTTTCTGGTGATCCT
CATTTGCTTGGATGGGGAGCTGGAACACCTTCAAGACCACTATTTTAA
Camelina Esc amino acid sequence (SEQ ID NO: 3):
MIQENLNTQTRIIINRVDPDSDPNTSSSTPGKRPRGRPPGSKNKAKPPIIVTRDSPNALRSH
VLEVSPGADIVESVSTYARRRGRGVSVLGGNGTVSNVTLRQPVTPGNGGGVSGGGGGG
VVTLHGRFEILSLTGTVLPPPAPPGAGGLSIFLAGGQGQVVGGSVVAPLIASAPVILMAA
SFSNAVFERLPMEEEEEEGAGAGGGGGGGPPQMQQAPSASPPSGVTGQGQLGGNVGG
YGFSGDPHLLGWGAGTPSRPLF*
Camelina sob3 cDNA sequence 873 bp (SEQ ID NO: 4; reverse
coding sequence):
CANANNGCGGNCANGAANGTGGATACTTTCACAACCTCTTTCAGACCTGACCTTCA
TCGCCAACTTCAACYTCAGCCTCATCTCCACCCTCTGCCTCAACCTCAACCTCAACC
TGAGCCTCAGCAACAACAATCAGATGATGAATCTGACTCCAACAAGGATCCGGGTT
CCGACCCGGTTACCTCGAGTTCAAACTCCTGGGAAGCGTCCACGTGGGCGTCCTCCG
GGATCTAAGAACAAGCCGAAGCCACCGGTGATAGTGACAAGAGATAGCCCCAACG
TGCTTAGATCTCATGTTCTTGAAATCTCATCTGGAGCCGACATAATTGAGTGCGTTA
ACACTTACGCTCGCCGGAGAGGGAAAGGTGTCTCCATTCTCAGTGGTAACGGCACG
GTAGCTAACGTCAGCATCCGTCAGCCGGCAACGGCTCATGCGACTAATGGTGGAGC
CGGAGGTGTTGTTTCTTTACATGGAAGGTTTGAGGTGCTTTCCATCACTGGTACGGT
GTTGCCACCACCTGCGCCCCCGGGATCCGGTGGTCTTTCTGTCTTTCTTGCCGGCAC
ACAAGGTCAGGTGGTCGGAGGACTCGCGGTGTCTCCGCTTGTGGCTTCGGGTCCAG
TGGTACTTATGGCTTCATCGTTCTCTAATGCAACTTTCGAACGGCTTCCGCTTGAGG
ATGAAGGAGGAGAAGGCGGAGGAGGAGAAGTTGGAGAGGGAGGTAGTGGAGCCG
GAGGTGGTGGTCCACCGCAGGCCACGTCGGCATCTTCACCACCGTCTGGAGCTGGT
CAAGGACAGTTAAGAGGTAACATGAGTGGTTATGATCAGTTTGCCGGTGATCCTCA
TGTGCTTGGTGGGAGCTCNGCCCTCCAGCC
Camelina Sob3 ORF 737 bp (SEQ ID NO: 5; reverse coding sequence):
ATGATGAATCTGACTCCAACAAGGATCCGGGTTCCGACCCGGTTACCTCGAGTTCA
AACTCCTGGGAAGCGTCCACGTGGGCGTCCTCCGGGATCTAAGAACAAGCCGAAGC
CACCGGTGATAGTGACAAGAGATAGCCCCAACGTGCTTAGATCTCATGTTCTTGAA
ATCTCATCTGGAGCCGACATAATTGAGTGCGTTAACACTTACGCTCGCCGGAGAGG
GAAAGGTGTCTCCATTCTCAGTGGTAACGGCACGGTAGCTAACGTCAGCATCCGTC
AGCCGGCAACGGCTCATGCGACTAATGGTGGAGCCGGAGGTGTTGTTTCTTTACAT
GGAAGGTTTGAGGTGCTTTCCATCACTGGTACGGTGTTGCCACCACCTGCGCCCCCG
GGATCCGGTGGTCTTTCTGTCTTTCTTGCCGGCACACAAGGTCAGGTGGTCGGAGGA
CTCGCGGTGTCTCCGCTTGTGGCTTCGGGTCCAGTGGTACTTATGGCTTCATCGTTCT
CTAATGCAACTTTCGAACGGCTTCCGCTTGAGGATGAAGGAGGAGAAGGCGGAGGA
GGAGAAGTTGGAGAGGGAGGTAGTGGAGCCGGAGGTGGTGGTCCACCGCAGGCCA
CGTCGGCATCTTCACCACCGTCTGGAGCTGGTCAAGGACAGTTAAGAGGTAACATG
AGTGGTTATGATCAGTTTGCCGGTGATCCTCATGTGCTTGGTGGGAGCTCNGCCCTC
CAGCC
Camelina Sob3 amino acid sequence (SEQ ID NO: 6):
MMNLTPTRIRVPTRLPRVQTPGKRPRGRPPGSKNKPKPPVIVTRDSPNVLRSHVLEISSG
ADIIECVNTYARRRGKGVSILSGNGTVANVSIRQPATAHATNGGAGGVVSLHGRFEVLS
ITGTVLPPPAPPGSGGLSVFLAGTQGQVVGGLAVSPLVASGPVVLMASSFSNATFERLPL
EDEGGEGGGGEVGEGGSGAGGGGPPQATSASSPPSGAGQGQLRGNMSGYDQFAGDPH
VLGGSSALQ*

According to additional aspects, Camelina AHL family polypeptides are provided that have at least one AT-hook motif/domain and a PPC domain (including c-terminal hydrophobic domain) (see above underlined exemplary AT-hook and PPC sequences in the Camelina Esc amino acid sequence (SEQ ID NO:3) and the Camelina Sob3 amino acid sequence (SEQ ID NO:6)), and wherein mutations of the AT hook domain confer a dominant negative phenotype as disclosed herein in the exemplary context of Arabidopsis thaliana AHL genes (Clade II and/or Clade I).

Particular aspects, therefore, relate to manipulation of the AT-hook domain in Camelina AHL polypeptides/AHL genes, including manipulation of the AT-hook domain (e.g., AT-hook domain mutants and modifications including but not limited to nonsense, missence, deletions, substitutions, muteins, fusions, etc.) in novel sequences SEQ ID NOS:1-6, which have substantial utility for modulation of cell growth in plants. Additional aspects relate to modified plants comprising Camelina derived AHL genes and gene products, and modified versions thereof.

According to yet further aspects, AHL family polypeptides of other plants, including but not limited to Oryza sativa (Rice); Sorghum bicolor (sorghum); and Zea mays (maize), Brassica rapa, Vitis vinifera, are provided that have at least one AT-hook motif/domain and a PPC domain, and wherein mutations of the at least one AT hook domain confer a dominant negative phenotype as disclosed herein in the exemplary context of Arabidopsis thaliana AHL genes (Clade II and/or Clade I) (see Tables 1 and 2 below).

TABLE 1
SOB3 and ESC Homologous Genes in Crops: Oryza sativa (Rice); Sorghum
bicolor (sorghum); and Zea mays (maize).
Oryza sativa (Rice)

| | Coding Sequence | | Protein Sequence | |
|---|---|---|---|---|
| Os02g0713700 | NM_001054449.2 | GI:297599833 | NP_001047914.1 | GI:115448269 |
| | SEQ ID NO: 63 | | SEQ ID NO: 64 | |
| Os06g0326900 | NM_001187833.1 | GI:297724796 | NP_001174762.1 | GI:297724797 |
| | SEQ ID NO: 65 | | SEQ ID NO: 66 | |
| Os02g0448000 | NM_001053289.2 | GI:297599146 | NP_001046754.1 | GI:115445949 |
| | SEQ ID NO:67 | | SEQ ID NO:68 | |

Sorghum bicolor (sorghum)

| | Coding Sequence | | Protein Sequence | |
|---|---|---|---|---|
| XM_002438296 | XM_002438296.1 | GI:242095701 | XP_002438341.1 | GI:242095702 |
| | SEQ ID NO: 69 | | SEQ ID NO: 70 | |
| XM_002452609 | XM_002452609.1 | GI:242062729 | XP_002452654.1 | GI:242062730 |
| | SEQ ID NO: 71 | | SEQ ID NO: 72 | |

Zea mays (maize)

| | Coding Sequence | | Protein Sequence | |
|---|---|---|---|---|
| NM_001157768 | NM_001157768.1 | GI:226502633 | NP_001151240.1 | GI:226502634 |
| | SEQ ID NO: 73 | | SEQ ID NO: 74 | |

TABLE 2
Arabidopsis thaliana AHL family member nucleic acid and protein
sequences.

| | | DNA | | Protein | |
|---|---|---|---|---|---|
| AHL | ATG | Accession | | Accession | |
| Number | Number | Number | GI Number | Number | GI Number |
| AHL1 | AT4G12080 | NM_117278.3 | GI:30682016 | NP_192945.2 | GI:22328578 |
| | | SEQ ID NO: 13 | | SEQ ID NO: 14 | |
| AHL2 | AT4G22770 | NM_118406.3 | GI:42567041 | NP_194008.1 | GI:15235790 |
| | | SEQ ID NO: 15 | | SEQ ID NO: 16 | |
| AHL3 | AT4G25320 | BT003408.1 | GI:28059576 | AAO30071.1 | GI:28059577 |
| | | SEQ ID NO: 17 | | SEQ ID NO: 18 | |
| AHL4 | AT5G51590 | NM_124538.3 | GI:42568466 | NP_1999721 | GI:15242131 |
| | | SEQ ID NO: 19 | | SEQ ID NO: 20 | |
| AHL5 | AT1G63470 | NM_105026.3 | GI:186492770 | NP_176536.2 | GI:30696854 |
| | | SEQ ID NO: 21 | | SEQ ID NO: 22 | |
| AHL6 | AT5G62260 | NM_125620.2 | GI:79544829 | NP_201032.2 | GI:79544830 |
| | | SEQ ID NO: 23 | | SEQ ID NO: 24 | |
| AHL7 | AT4G00200 | NM_116237.3 | GI:145339838 | NP_191931.2 | GI:145339839 |
| | | SEQ ID NO: 25 | | SEQ ID NO: 26 | |
| AHL8 | AT5G46640 | BT015755.1 | GI:52627130 | AAU84692.1 | GI:52627131 |
| | | SEQ ID NO: 27 | | SEQ ID NO: 28 | |
| AHL9 | AT2G45850 | AY114678.1 | GI:21387186 | AAM47997.1 | GI:21387187 |
| | | SEQ ID NO: 29 | | SEQ ID NO: 30 | |
| AHL10 | AT2G33620 | NM_001124965.1 | GI:186505051 | NP_001118437.1 | GI:186505052 |
| | | SEQ ID NO: 31 | | SEQ ID NO: 32 | |
| AHL11 | AT3G61310 | BT008837.1 | GI:31711839 | AAP68276.1 | GI:31711840 |
| | | SEQ ID NO: 33 | | SEQ ID NO: 34 | |
| AHL12 | AT1G63480 | AY096576.1 | GI:20466008 | AAM20226.1 | GI:20466009 |
| | | SEQ ID NO: 35 | | SEQ ID NO: 36 | |
| AHL13 | AT4G17950 | AY081495.1 | GI:20148332 | AAM10057.1 | GI:20148333 |
| | | SEQ ID NO: 37 | | SEQ ID NO: 38 | |
| AHL14 | AT3G04590 | NM_180181.3 | GI:79596509 | NP 850512.2 | GI:79596510 |
| | | SEQ ID NO: 39 | | SEQ ID NO: 40 | |
| AHL15 | AT3G55560 | BT024777.1 | GI:89001050 | ABD59115.1 | GI:89001051 |
| | | SEQ ID NO: 41 | | SEQ ID NO: 42 | |
| AHL16 | AT2G42940 | BT010995.1 | GI:38604059 | AAR24773.1 | GI:38604060 |
| | | SEQ ID NO: 42 | | SEQ ID NO: 42 | |
| AHL17 | AT5G49700 | NM_124348.1 | GI:18423057 | NP_199781.1 | GI:15240535 |
| | | SEQ ID NO: 43 | | SEQ ID NO: 44 | |
| AHL18 | AT3G60870 | NM_115951.1 | GI:18411756 | NP_191646.1 | GI:15232970 |
| | | SEQ ID NO: 45 | | SEQ ID NO: 46 | |
| AHL19 | AT3G04570 | BT005882.1 | GI:29028875 | AAO64817.1 | GI:29028876 |
| | | SEQ ID NO: 47 | | SEQ ID NO: 48 | |
| AHL20 | AT4G14465 | NM_111328.2 | GI:30679174 | NP_566232.1 | GI:18396925 |
| | | SEQ ID NO: 49 | | SEQ ID NO: 50 | |
| AHL21 | AT2G35270 | NM_129079.1 | GI:18403788 | NP_181070.1 | GI:15226945 |
| | | SEQ ID NO: 51 | | SEQ ID NO: 52 | |
| AHL22 | AT2G45430 | BT020250.1 | GI:56121925 | AAV74244.1 | GI:56121926 |
| | | SEQ ID NO: 53 | | SEQ ID NO: 54 | |
| AHL23 | AT4G17800 | NM_117890.4 | GI:42566907 | NP_193515.1 | GI:15236657 |
| | | SEQ ID NO: 55 | | SEQ ID NO: 56 | |
| AHL24 | AT4G22810 | NM_118410.2 | GI:30685940 | NP_194012.1 | GI:15235815 |
| | | SEQ ID NO: 57 | | SEQ ID NO: 58 | |
| AHL25 | AT4G35390 | BT014971.1 | GI:50198776 | AAT70422.1 | GI:50198777 |
| | | SEQ ID NO: 11 | | SEQ ID NO: 12 | |
| AHL26 | AT4G12050 | NM_117275.3 | GI:145340130 | NP_192942.1 | GI:15234404 |
| | | SEQ ID NO: 59 | | SEQ ID NO: 60 | |
| AHL27 | AT1G20900 | BT006460.1 | GI:30102699 | AAP21268.1 | GI:30102700 |
| | | SEQ ID NO: 7 | | SEQ ID NO: 8 | |
| AHL28 | AT1G14490 | BT029503.1 | GI:119360060 | ABL66759.1 | GI:119360061 |
| | | SEQ ID NO: 61 | | SEQ ID NO: 62 | |
| AHL29 | AT1G76500 | NM_106300.3 | GI:145337635 | NP_177776.1 | GI:15223074 |
| | | SEQ ID NO: 9 | | SEQ ID NO: 10 | |

### Example 6

### Expression of the Atsob3-6 cDNA (and other Similar AHL Mutations) were used to Alter Plant Cell Growth

Over-expression of Atsob3-6 cDNA in Arabidopsis demonstrates that this allele acts as a dominant-negative allele to enhance hypocotyl elongation in seedlings.

According to particular aspects, over-expression of this dominant-negative allele were also shown to affect adult growth and development suggesting that these types of alleles can be used for altering plant cell growth in general (FIG. 2).

According to particular aspects, it is likely that this phenotype is caused by interaction between SOB3 and other members of this protein family such as ESC and those that are co-expressed with these two genes (FIG. 6).

Co-expression analysis of AHL family members Twenty-five of the 29 Arabidopsis AHL genes are expressed in hypocotyls based on e-northern analysis using Affymetrix ATH1 microarray. However, no corresponding probe sets exist for the other four AHL genes. Coexpressed gene information of AHL members has been retrieved from ATTED-II (Arabidopsis thaliana trans-factor and cis-element prediction database)(Obayashi et al., 2007; Obayashi et al., 2009). Various AHL members have been identified as components in co-expression networks. With this information we generated a network of AHL members with a correlation of co-expression. (FIG. 6B). Gene clustering analysis via the BAR (The Bio-Array Resource for Arabidopsis functional genomics) database based on current publicly available microarray data also suggests that the expression of these AHL gene family members are tightly related with each other (FIG. 6C; Toufighi et al., 2005). Therefore, among the 29 AHL genes encoded in Arabidopsis thaliana genome, these members are the best candidates for functional redundancy with SOB3 and ESC in seedling and adult plant development; for example, the subset of AHL genes: AHL19, AHL21, AHL22, AHL23 and AHL6, that locate within the co-expressed network I (FIG. 6), which can be expanded to include AHL1, AHL18 and AHL25 (Fujimoto et al., 2004; Jiang, 2004; Lim et al., 2007; Xiao et al., 2009).

According to particular aspects, another interaction is with the next closest family member HRC (FIGS. 6 and 7).

According to additional aspects, Applicants have now shown that SOB3, ESC and HRC can physically interact (FIGS. 3 and 4).

### Example 8

### Transgenic Camelina Plants Expressing the Atsob3-6 cDNA were shown to have Longer Hypocotyls than the Wild Type Controls

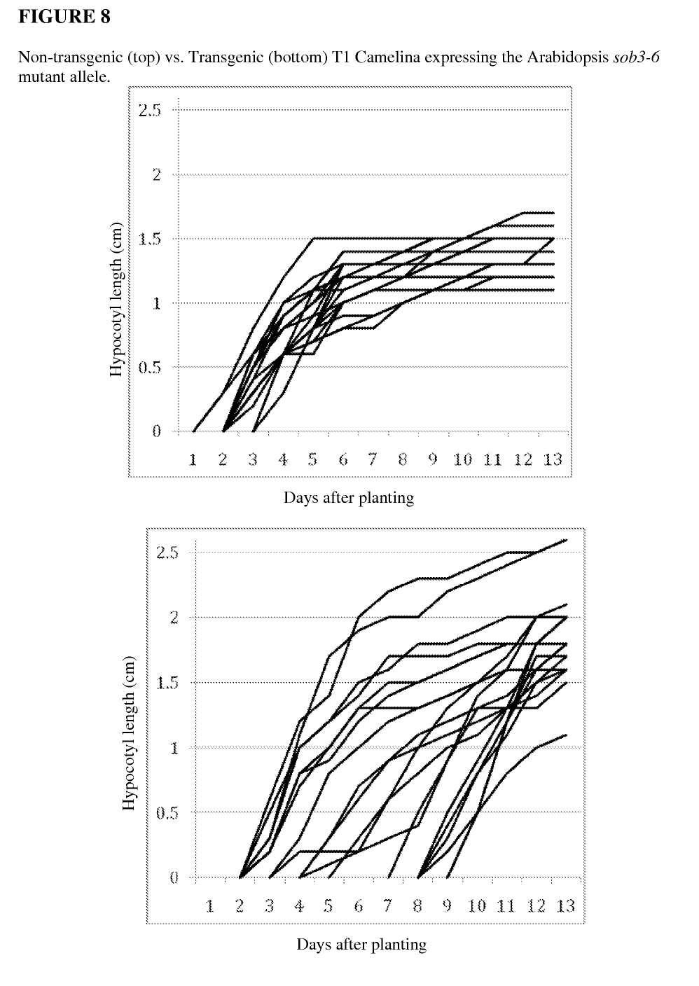

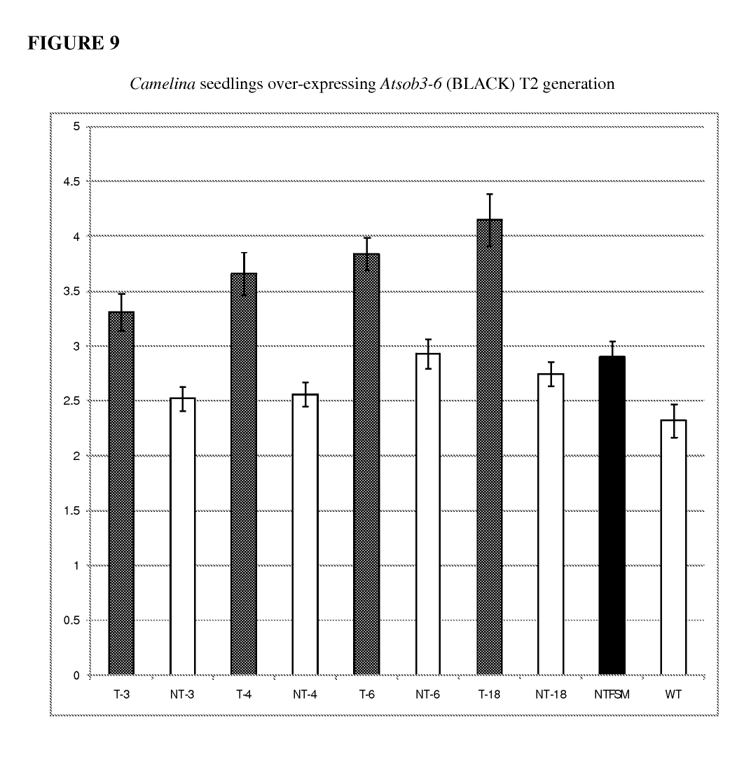

According to further aspects, Applicants have now shown that transgenic Camelina plants expressing the Atsob3-6 cDNA have longer hypocotyls than the wild type controls for both primary (T1) transformants (FIG. 8) and T2 plants in the next generation (FIG. 9).

### Example 9

### Camelina Sob3 and Esc Sequences/Proteins have Utility to Modulate Cell Growth in Plants

According to further aspects, the novel sequences (SEQ ID NOS:1-6) shown in Example 5 above, have substantial utility for modulation of cell growth in plants.

According to further aspects, manipulation of the AT-hook domain in Camelina AHL genes, including manipulation of the AT-hook domain in the novel sequences (SEQ ID NOS:1-6) shown in Example 5 above, have substantial utility for modulation of cell growth in plants. For example AT-hook domain mutants and modification (e.g., nonsense, missence, deletions, substitutions, muteins, fusions, etc.) of SEQ ID NOS:1-6 have substantial utility for modulation of cell growth in plants.

### Example 10

### Antibodies are Raised Against Camelina SOB3 and ESC Proteins

According to further aspects, antibodies are raised against the Camelina SOB3 and ESC proteins using the service from Open Biosystem, Inc®. Due to the high similarity of SOB3 and ESC at the protein level (e.g., over 89%), antibodies are developed specifically against peptides from divergent regions in their C-termini. For SOB3 and ESC, the synthetic peptides ‘RGNMSGYDQFAGDPHL’ and ‘CLGWGAGTPSRPPF’ (including Camelina counterparts) are used, respectively. Antibody specificities can be confirmed using E.coli synthesized recombinant proteins. These gene-specific antibodies are used to confirm, for example, that SOB3 and ESC associate with each other by in vitro pull-down assays.

### Example 11

### Genetic Manipulation of the AT-Hook Domain in Plant AHL Genes to Modulate Cell Growth

According to further aspects, non-GM breeding approaches, as widely recognized in the art, are used for manipulation of the AT-hook domain in plant AHL genes to modulate cell growth.

### Example 12

### Targeted Expression of PPC/DUF Domains in Plant AHL Genes to Modulate Cell Growth

According to further aspects, targeted expression of PPC/DUF domains in plant AHL genes has utility for modulating plant cell growth. In particular aspects, targeted expression of PPC/DUF domains and including the spacer region between the PPC domain and the AT-hook domain, has utility for modulating plant cell growth.

### Example 13

### Additional Exemplary AHL Sequences

Arabidopsis thaliana ESC (ESCAROLA) (ESC) mRNA, complete cds
(SEQ ID NO: 7):
GGACCAAAAATTTATTGCAGAGTCGCACATGAATCTCAAGCTTCTCTCTCCTTTTTTT
CCCATAGCACATCAGAATCGCTAAATACGACTCCTATGCAAAGAAGAAGCTACTTC
TTTCTCTTGCCCTAATTAATCTACCTAACTAGGGTTTCCTCTTACCTTTCATGAGAGA
GATCATTTAACATAAGTCACCTTTTTTATATCTTTTGCTTCGTCTTTAATTTAGTTCTG
TTCTTGGTCTGTTTCTATATTTTGTCGGCTTGCGTAACCGATCACACCTTAATGCTTT
AGCTATTGTTTCCTCAAAATCATGAGTTTTGACTTCTCGATCTGAGTTTTCTTTTTCT
CTCTTTACGCTCTTCTTCACCTAGCTACCAATATATGAACGAGCAGGATCAAGAATC
GAGAAATTGATTTGAGCTGGCGAATAAGCAGTGGTGGGATAGGGAATTAGTAGATG
CGGCGGCGATGGAAGGCGGTTACGAGCAAGGCGGTGGAGCTTCTAGATACTTCCAT
AACCTCTTTAGACCGGAGATTCACCACCAACAGCTTCAACCGCAGGGCGGGATCAA
TCTTATCGACCAGCATCATCATCAGCACCAGCAACATCAACAACAACAACAACCGT
CGGATGATTCAAGAGAATCTGACCATTCAAACAAAGATCATCATCAACAGGGTCGA
CCCGATTCAGACCCGAATACATCAAGCTCAGCACCGGGAAAACGTCCACGTGGACG
TCCACCAGGATCTAAGAACAAAGCCAAGCCACCGATCATAGTAACTCGTGATAGCC
CCAACGCGCTTAGATCTCACGTTCTTGAAGTATCTCCTGGAGCTGACATAGTTGAGA
GTGTTTCCACGTACGCTAGGAGGAGAGGGAGAGGCGTCTCCGTTTTAGGAGGAAAC
GGCACCGTATCTAACGTCACTCTCCGTCAGCCAGTCACTCCTGGAAATGGCGGTGGT
GTGTCCGGAGGAGGAGGAGTTGTGACTTTACATGGAAGGTTTGAGATTCTTTCGCTA
ACGGGGACTGTTTTGCCACCTCCTGCACCGCCTGGTGCCGGTGGTTTGTCTATATTTT
TAGCCGGAGGGCAAGGTCAGGTGGTCGGAGGAAGCGTTGTGGCTCCCCTTATTGCA
TCAGCTCCGGTTATACTAATGGCGGCTTCGTTCTCAAATGCGGTTTTCGAGAGACTA
CCGATTGAGGAGGAGGAAGAAGAAGGTGGTGGTGGCGGAGGAGGAGGAGGAGGA
GGGCCACCGCAGATGCAACAAGCTCCATCAGCATCTCCGCCGTCTGGAGTGACCGG
TCAGGGACAGTTAGGAGGTAATGTGGGTGGTTATGGGTTTTCTGGTGATCCTCATTT
GCTTGGATGGGGAGCTGGAACACCTTCAAGACCACCTTTTTAATTGAATTTTAATGT
CCGGAAATTTATGTGTTTTTATCATCTTGTGGAGTCGTCTTTCCTTTGGGATATTTGG
TGTTTAATGTTTAGTTGATATGCATATTTTGGTTTCTCGTG
Arabidopsis thaliana ESC putative protein (SEQ ID NO: 8):
MEGGYEQGGGASRYFHNLFRPEIHHQQLQPQGGINLIDQHHHQHQQHQQQQQPSDDSR
ESDHSNKDHHQQGRPDSDPNTSSSAPGKRPRGRPPGSKNKAKPPIIVTRDSPNALRSHVL
EVSPGADIVESVSTYARRRGRGVSVLGGNGTVSNVTLRQPVTPGNGGGVSGGGGVVTL
HGRFEILSLTGTVLPPPAPPGAGGLSIFLAGGQGQVVGGSVVAPLIASAPVILMAASFSN
AVFERLPIEEEEEEGGGGGGGGGGGPPQMQQAPSASPPSGVTGQGQLGGNVGGYGFSG
DPHLLGWGAGTPSRPPF
Arabidopsis thaliana DNA-binding family protein (AT1G76500) mRNA,
complete cds (SEQ ID NO: 9):
CTGCCATGGACGGTGGTTACGATCAATCCGGAGGAGCTTCTAGATACTTTCACAACC
TCTTCAGGCCTGAGCTTCATCACCAGCTTCAACCTCAGCCTCAACTTCACCCTTTGCC
TCAGCCTCAGCCTCAACCTCAGCCTCAGCAGCAGAATTCAGATGATGAATCTGACTC
CAACAAGGATCCGGGTTCCGACCCAGTTACCTCTGGTTCAACCGGGAAACGTCCAC
GTGGACGTCCTCCGGGATCCAAGAACAAGCCGAAGCCACCGGTGATAGTGACTAGA
GATAGCCCCAACGTGCTTAGATCTCATGTTCTTGAAGTCTCATCTGGAGCCGACATA
GTCGAGAGCGTTACCACTTACGCTCGCAGGAGAGGAAGAGGAGTCTCCATTCTCAG
TGGTAACGGCACGGTGGCTAACGTCAGTCTCCGGCAGCCGGCAACGACAGCGGCTC
ATGGGGCAAATGGTGGAACCGGAGGTGTTGTGGCTCTACATGGAAGGTTTGAGATA
CTTTCCCTCACAGGTACGGTGTTGCCGCCCCCTGCGCCGCCAGGATCCGGTGGTCTT
TCTATCTTTCTTTCCGGCGTTCAAGGTCAGGTGATTGGAGGAAACGTGGTGGCTCCG
CTTGTGGCTTCGGGTCCAGTGATACTAATGGCTGCATCGTTCTCTAATGCAACTTTC
GAAAGGCTTCCCCTTGAAGATGAAGGAGGAGAAGGTGGAGAGGGAGGAGAAGTTG
GAGAGGGAGGAGGAGGAGAAGGTGGTCCACCGCCGGCCACGTCATCATCACCACC
ATCTGGAGCCGGTCAAGGACAGTTAAGAGGTAACATGAGTGGTTATGATCAGTTTG
CCGGTGATCCTCATTTGCTTGGTTGGGGAGCCGCAGCCGCAGCCGCACCACCAAGA
CCAGCCTTTTAGAATTGAAAATTATGTCCGTAACATAGCTGTAACCAAATTTCATTT
CTCAAAATTAAAAGAAAAAAAAAATCATCTTCATTGTTTGGGGATCGTTTGGTTTTT
AATTTAGTTGATCATATATG
Arabidopsis thaliana Sob3 putative protein (SEQ ID NO: 10)
MDGGYDQSGGASRYFHNLFRPELHHQLQPQPQLHPLPQPQPQPQPQQQNSDDESDSNK
DPGSDPVTSGSTGKRPRGRPPGSKNKPKPPVIVTRDSPNVLRSHVLEVSSGADIVESVTT
YARRRGRGVSILSGNGTVANVSLRQPATTAAHGANGGTGGVVALHGRFEILSLTGTVL
PPPAPPGSGGLSIFLSGVQGQVIGGNVVAPLVASGPVILMAASFSNATFERLPLEDEGGE
GGEGGEVGEGGGGEGGPPPATSSSPPSGAGQGQLRGNMSGYDQFAGDPHLLGWGAAA
AAAPPRPAF
Camelina ESC (SEQ ID NO: 1)
AATTCNGGANTTTAANTTCGATTAAAATAGTGGTCTTGAAGGTGTTCCAGCTCCCCA
TCCAAGCAAATGAGGATCACCAGAAAACCCATAACCACCCACATTACCTCCTAACT
GTCCCTGACCGGTCACGCCTGACGGAGGCGATGCTGAGGGAGCTTGCTGCATCTGC
GGTGGTCCTCCTCCTCCCCCTCCGCCAGCACCAGCACCTTCTTCTTCTTCCTCTTCCA
TTGGTAGTCTCTCGAAAACCGCATTTGAGAACGAAGCAGCCATTAGTATAACTGGA
GCTGATGCAATAAGCGGAGCCACCACGCTTCCTCCAACAACCTGACCTTGCCCACC
GGCTAGAAATATTGACAAACCACCTGCACCAGGCGGCGCAGGAGGTGGCAAAACA
GTCCCCGTTAGTGAAAGAATCTCAAATCTTCCATGTAAAGTCACAACTCCTCCTCCT
CCTCCTCCGGACACACCACCACCGTTTCCGGGAGTGACTGGCTGACGGAGAGTGAC
GTTAGAAACGGTGCCGTTCCCTCCTAAAACGGAGACGCCTCTCCCTCTCCGCCTAGC
GTAAGTGGAAACACTCTCAACTATATCAGCTCCGGGAGATACTTCAAGGACGTGAG
ATCTAAGCGCGTTGGGGCTGTCACGGGTTACTATGATCGGTGGCTTTGCTTTGTTCT
TAGATCCTGGCGGACGTCCACGTGGACGTTTCCCGGGTGTTGAGCTTGATGTATTCG
GGTCTGAATCGGGGTCGACCCTGTTGATGATGATCCTTGTTTGAGTGTTCAGATTCT
CTTGAATCATCCGACGGTKGCTGTTGTTGTTGTTGTTGTTGCTGCTGATGCTGATGAT
GATGCTGGTCAATAAGATTGATTCCACCCTGCGGTTGAAGGTGCTGGTGGTGAATCT
CCGGTCTGAAGAGGTTTAGAAAGATCTAGAAGTCCACCGCTNTA
Camelina Esc ORF (SEQ ID NO: 2)
ATGATTCAAGAGAATCTGAACACTCAAACAAGGATCATCATCAACAGGGTCGACCC
CGATTCAGACCCGAATACATCAAGCTCAACACCCGGGAAACGTCCACGTGGACGTC
CGCCAGGATCTAAGAACAAAGCAAAGCCACCGATCATAGTAACCCGTGACAGCCCC
AACGCGCTTAGATCTCACGTCCTTGAAGTATCTCCCGGAGCTGATATAGTTGAGAGT
GTTTCCACTTACGCTAGGCGGAGAGGGAGAGGCGTCTCCGTTTTAGGAGGGAACGG
CACCGTTTCTAACGTCACTCTCCGTCAGCCAGTCACTCCCGGAAACGGTGGTGGTGT
GTCCGGAGGAGGAGGAGGAGGAGTTGTGACTTTACATGGAAGATTTGAGATTCTTT
CACTAACGGGGACTGTTTTGCCACCTCCTGCGCCGCCTGGTGCAGGTGGTTTGTCAA
TATTTCTAGCCGGTGGGCAAGGTCAGGTTGTTGGAGGAAGCGTGGTGGCTCCGCTT
ATTGCATCAGCTCCAGTTATACTAATGGCTGCTTCGTTCTCAAATGCGGTTTTCGAG
AGACTACCAATGGAAGAGGAAGAAGAAGAAGGTGCTGGTGCTGGCGGAGGGGGAG
GAGGAGGACCACCGCAGATGCAGCAAGCTCCCTCAGCATCGCCTCCGTCAGGCGTG
ACCGGTCAGGGACAGTTAGGAGGTAATGTGGGTGGTTATGGGTTTTCTGGTGATCCT
CATTTGCTTGGATGGGGAGCTGGAACACCTTCAAGACCACTATTTTAA
Camelina putative Esc amino acid sequence (SEQ ID NO: 3)
MIQENLNTQTRIIINRVDPDSDPNTSSSTPGKRPRGRPPGSKNKAKPPIIVTRDSPNALRSH
VLEVSPGADIVESVSTYARRRGRGVSVLGGNGTVSNVTLRQPVTPGNGGGVSGGGGGG
VVTLHGRFEILSLTGTVLPPPAPPGAGGLSIFLAGGQGQVVGGSVVAPLIASAPVILMAA
SFSNAVFERLPMEEEEEEGAGAGGGGGGGPPQMQQAPSASPPSGVTGQGQLGGNVGG
YGFSGDPHLLGWGAGTPSRPLF
Camelina sob3 (SEQ ID NO: 4)
GGCTGGAGGGCNGAGCTCCCACCAAGCACATGAGGATCACCGGCAAACTGATCATA
ACCACTCATGTTACCTCTTAACTGTCCTTGACCAGCTCCAGACGGTGGTGAAGATGC
CGACGTGGCCTGCGGTGGACCACCACCTCCGGCTCCACTACCTCCCTCTCCAACTTC
TCCTCCTCCGCCTTCTCCTCCTTCATCCTCAAGCGGAAGCCGTTCGAAAGTTGCATTA
GAGAACGATGAAGCCATAAGTACCACTGGACCCGAAGCCACAAGCGGAGACACCG
CGAGTCCTCCGACCACCTGACCTTGTGTGCCGGCAAGAAAGACAGAAAGACCACCG
GATCCCGGGGGCGCAGGTGGTGGCAACACCGTACCAGTGATGGAAAGCACCTCAA
ACCTTCCATGTAAAGAAACAACACCTCCGGCTCCACCATTAGTCGCATGAGCCGTTG
CCGGCTGACGGATGCTGACGTTAGCTACCGTGCCGTTACCACTGAGAATGGAGACA
CCTTTCCCTCTCCGGCGAGCGTAAGTGTTAACGCACTCAATTATGTCGGCTCCAGAT
GAGATTTCAAGAACATGAGATCTAAGCACGTTGGGGCTATCTCTTGTCACTATCACC
GGTGGCTTCGGCTTGTTCTTAGATCCCGGAGGACGCCCACGTGGACGCTTCCCAGGA
GTTTGAACTCGAGGTAACCGGGTCGGAACCCGGATCCTTGTTGGAGTCAGATTCATC
ATCTGATTGTTGTTGCTGAGGCTCAGGTTGAGGTTGAGGTTGAGGCAGAGGGTGGA
GATGAGGCTGARGTTGAAGTTGGCGATGAAGGTCAGGTCTGAAAGAGGTTGTGAAA
GTATCCACNTTCNTGNCCGCNNTNTG
Camelina Sob3 ORF (SEQ ID NO: 5)
GGCTGGAGGGCNGAGCTCCCACCAAGCACATGAGGATCACCGGCAAACTGATCATA
ACCACTCATGTTACCTCTTAACTGTCCTTGACCAGCTCCAGACGGTGGTGAAGATGC
CGACGTGGCCTGCGGTGGACCACCACCTCCGGCTCCACTACCTCCCTCTCCAACTTC
TCCTCCTCCGCCTTCTCCTCCTTCATCCTCAAGCGGAAGCCGTTCGAAAGTTGCATTA
GAGAACGATGAAGCCATAAGTACCACTGGACCCGAAGCCACAAGCGGAGACACCG
CGAGTCCTCCGACCACCOTGACCTTGTGTGCCGGCAAGA AAGACAGAAAGACCACC
GGATCCCGGGGGCGCAGGTGGTGGCAACACCGTACCAGTGATGGAAAGCACCTCA
AACCTTCCATGTAAAGAAACAACACCTCCGGCTCCACCATTAGTCGCATGAGCCGTT
GCCGGCTGACGGATGCTGACGTTAGCTACCGTGCCGTTACCACTGAGAATGGAGAC
ACCTTTCCCTCTCCGGCGAGCGTAAGTGTTAACGCACTCAATTATGTCGGCTCCAGA
TGAGATTTCAAGAACATGAGATCTAAGCACGTTGGGGCTATCTCTTGTCACTATCAC
CGGTGGCTTCGGCTTGTTCTTAGATCCCGGAGGACGCCCACGTGGACGCTTCCCAGG
AGTTTGAACTCGAGGTAACCGGGTCGGAACCCGGATCCTTGTTGGAGTCAGATTCA
TCAT
Camelina Sob3 putative amino acid sequence (SEQ ID NO: 6):
MMNLTPTRIRVPTRLPRVQTPGKRPRGRPPGSKNKPKPPVIVTRDSPNVLRSHVLEISSG
ADIIECVNTYARRRGKGVSILSGNGTVANVSIRQPATAHATNGGAGGVVSLHGRFEVLS
ITGTVLPPPAPPGSGGLSVFLAGTQGQVVGGLAVSPLVASGPVVLMASSFSNATFERLPL
EDEGGEGGGGEVGEGGSGAGGGGPPQATSASSPPSGAGQGQLRGNMSGYDQFAGDPH
VLGGSSALQ
Sob 3 homologue in Brassica rapa sub Pekinese: [mRNA] 1 exon (s)
87-950 864 bp, chain +
(SEQ ID NO: 76):
ATGGACGGTGGTTATGATCAATCCGGTCACTCTAGATACTTCCATAACCTCTTTAGG
CCTGAGCTTCAACACCAGCTTCAGCCACAGCCGCAGCCTCAACCCCAGCCTCAGCCT
CAGCCTCAGCCTCAGTCTGATGATGAATCTGACTCCAACAACAAGTATCCGGGTCA
ACCTGATTCCGACCAGGTTACCTCGGGCTCAACTTCCGGGAAGCGTCCACGTGGAC
GTCCTCCAGGGTCTAAGAACAAGCCGAAGCCACCGGTGATAGTGACAAGAGATAGC
CCCAACGTGCTTAGATCTCATGTTCTTGAAGTCTCATCTGGAGCCGACATAATTGAG
AGCGTCAACAATTATGCTCGCCGGAGAGGGAGAGGTGTCTCCATTCTCAGTGGTAA
CGGCACGGTGGCTAACCTCACTCTCCGGCAGCCGGTGACGACTCATGGGAACAATG
GTGGAACTGAAGCCGGAGCCGGAGGAGTTGTGACTTTACGTGGAAGGTTTGAGATT
CTTTCCATCACTGGTACGGTGCTTCCGCCGCCCGCGCCGCCGGGATGCGGTGGTTTA
TCTATCTTTGTTGCTGGTGAACAAGGTCGGGTGATCGGAGGAAGAGTGGTGGCTCC
CCTTGTGGCTTCTGGTCCAGTGATACTGATGGCTGCATCGTTCTCCAACGCAACTTT
CGAAAGGCTTCCACTTGAAGAGGAGGGAGGTGAAGGTGGGGGAGACGTCGGAGGA
GGAGTTCCACCGCCAGCCACTTCAGAAACAGCGCCGTCTGGAGTCGCTCAGGGAGA
GCTAAGAGTTAATATGAGTGGTTATGATCAGTTTTCCGGCTGGGGAGCCGGAGCCG
CTTCAAGACCATCATTTTAG
Sob 3 homologue in Brassica rapa sub Pekinese: putative amino acid
sequence 1 exon (s) 87-950 287 aa, chain + (SEQ ID NO: 77):
MDGGYDQSGHSRYFHNLFRPELQHQLQPQPQPQPQPQPQPQPQSDDESDSNNKYPGQP
DSDQVTSGSTSGKRPRGRPPGSKNKPKPPVIVTRDSPNVLRSHVLEVSSGADIIESVNNY
ARRRGRGVSILSGNGTVANLTLRQPVTTHGNNGGTEAGAGGVVTLRGRFEILSITGTVL
PPPAPPGCGGLSIFVAGEQGRVIGGRVVAPLVASGPVILMAASFSNATFERLPLEEEGGE
GGGDVGGGVPPPATSETAPSGVAQGELRVNMSGYDQFSGWGAGAASRPSF
Esc homologue in Brassica rapa sub. Pekinese: [mRNA] 1 exon (s)
67-1002 936 bp, chain + (SEQ ID NO: 78):
ATGGAAGGCGGCTACGAGCAAGGCGGTGGAGCTTCTAGGTACTTCCATAACCTCTT
CAGACCAGAGATTCACCACCAACAGCTTCAACAACAAGGCGGGATCAATCTTTTTG
ACCAGCATCATCAACAGCAACAACATCAGCAGCAACAACAACAACAACCGTCAGA
TGATTCAAGAGAATCCGATCACTCAAACAAGGATCATCATCAACCGGGTCTACCCG
ATTCAGACCCGGCTACATCAAGCTCAGCACCTGGGAAACGTCCACGTGGACGTCCA
CCGGGATCTAAGAACAAAGCTAAGCCACCGATCATAGTGACGCGGGACAGCCCCA
ATGCGCTTAGATCTCACGTCCTTGAAGTATCTCCTGGAGCTGACATAGTTGAGTGTG
TGTCCACTTACGCTAGGCGGAGAGGGAGAGGGGTCTCCGTTTTAGGAGGAAACGGC
ACCGTTTCCAACGTCACTCTTCGTCAGCCAGTCACTCCCGGAAATAGCGGTGGTGGA
GCCGGAGGAGGAGTTGTGACTTTACATGGAAGGTTTGAGATTCTTTCGCTAACGGG
AACCGTTTTGCCACCACCTGCACCGCCAGGTGCTGGTGGTTTGTCAATATTTTTATC
CGGAGGGCAAGGTCAGGTGGTTGGGGGAAGCGTTGTGGCTCCGCTTGTTGCATCAG
CTCCGGTTATACTAATGGCTGCTTCCTTCTCAAACGCGGTTTTCGAGAGATTGCCTA
TTGAAGAGGAGGAAGAAAGAGGTGGTGGCGGTGTAGGAGAAGGAGAAGGACCACC
GCAGATGCAGCAAGCTCCATCACCATCTCCGCGGTCGGGGGTGACCGGTCAAGGAC
AGCTAGGAGGTAATGTGGGTGGTTATGGGTTTTCCAGTGATCCTCATTTGCTAGGAT
GGGGAGCTGGTACGCCTTCAAGACCACCTTTTACTTAA
Esc homologue in Brassica rapa sub. Pekinese: putative amino acid
sequence 1 exon (s) 67-1002 311 aa, chain + (SEQ ID NO: 79):
MEGGYEQGGGASRYFHNLFRPEIHHQQLQQQGGINLFDQHHQQQQHQQQQQQQPSDD
SRESDHSNKDHHQPGLPDSDPATSSSAPGKRPRGRPPGSKNKAKPPIIVTRDSPNALRSH
VLEVSPGADIVECVSTYARRRGRGVSVLGGNGTVSNVTLRQPVTPGNSGGGAGGGVVT
LHGRFEILSLTGTVLPPPAPPGAGGLSIFLSGGQGQVVGGSVVAPLVASAPVILMAASFS
NAVFERLPIEEEEERGGGGVGEGEGPPQMQQAPSPSPRSGVTGQGQLGGNVGGYGFSS
DPHLLGWGAGTPSRPPFT
DNA-binding family protein [Arabidopsis thaliana] (sob3)
(SEQ ID NO: 10):
MDGGYDQSGGASRYFHNLFRPELHHQLQPQPQLHPLPQPQPQPQPQQQNSDDESDSNK
DPGSDPVTSGSTGKRPRGRPPGSKNKPKPPVIVTRDSPNVLRSHVLEVSSGADIVESVTT
YARRRGRGVSILSGNGTVANVSLRQPATTAAHGANGGTGGVVALHGRFEILSLTGTVL
PPPAPPGSGGLSIFLSGVQGQVIGGNVVAPLVASGPVILMAASFSNATFERLPLEDEGGE
GGEGGEVGEGGGGEGGPPPATSSSPPSGAGQGQLRGNMSGYDQFAGDPHLLGWGAAA
AAAPPRPAF
ESC (ESCAROLA) [Arabidopsis thaliana] (SEQ ID NO: 8):
MEGGYEQGGGASRYFHNLFRPEIHHQQLQPQGGINLIDQHHHQHQQHQQQQQPSDDSR
ESDHSNKDHHQQGRPDSDPNTSSSAPGKRPRGRPPGSKNKAKPPIIVTRDSPNALRSHVL
EVSPGADIVESVSTYARRRGRGVSVLGGNGTVSNVTLRQPVTPGNGGGVSGGGGVVTL
HGRFEILSLTGTVLPPPAPPGAGGLSIFLAGGQGQVVGGSVVAPLIASAPVILMAASFSN
AVFERLPIEEEEEEGGGGGGGGGGGPPQMQQAPSASPPSGVTGQGQLGGNVGGYGFSG
DPHLLGWGAGTPSRPPF
PREDICTED: hypothetical protein [Vitis vinifera] (SEQ ID NO: 80):
MAGMEQGAGSRYIHQLFRPELQLERTPQQPHQPPQLNDSGDSPENEDRTDPDGSPGAA
TTSSRRPRGRPPGSKNKAKPPIIITRDSPNALRSHVLEISAGADIVESVSNYARRRGRGVCI
LSGGGAVTDVTLRQPAAPSGSVVTLHGRFEILSLTGTALPPPAPPGAGGLTIYLGGGQGQ
VVGGRVVGPLVASGPVLLMAASFANAVYDRLPLEEEEESPVQVQPTASQSSGVTGGGG
QLGDGGNGSTTTAGGGAGAGVPFYNLGPNMGNYPFPGDVFGWNGGATRPPF
PREDICTED: hypothetical protein [Vitis vinifera] (SEQ ID NO: 81):
MEGYEPGSGSRYVHQLLGPELHLQRPSSLPQHQATQQPSDSRDESPDDQEQRADTEEA
AAASSGGATTSSNRRPRGRPPGSKNKPKPPIIVTRDSPNALRSHVLEVAAGADVMESVL
NYARRRGRGVCVLSGGGTVMNVTLRQPASPAGSIVTLHGRFEILSLSGTVLPPPAPPSAG
GLSIFLSGGQGQVVGGSVVGPLMASGPVVLMAASFANAVFERLPLEEEEGAVQVQPTA
SQSSGVTGGGAGGQLGDGGGSGGGAGVPIYNMGASMGNFPFPGDLLRWGGSAPRPPF
DNA binding protein, putative [Ricinus communis] (SEQ ID NO: 25):
MAGYNNEQSATGTGSRYVHQLLRPELHLQRPSFPSQPSSDSKDNNISPQSKDHNKFSDS
EAAAATSSGSNRRPRGRPAGSKNKPKPPIIVTRDSPNALRSHVLEVSTGSDIMESVSIYAR
KRGRGVCVLSGNGTVANVTLRQPASPAGSVVTLHGRFEILSLSGTVLPPPAPPGAGGLSI
FLSGGQGQVVGGSVVGPLMASGPVVLMAASFANAVFERLPLDEEDGTVPVQSTASQSS
GVTGGGGGAGQLGDGGGGGGAGLFNMGGNVANYPFSGDLFGW GVNAARPPF
Oryza sativa (japonica cultivar-group) (SEQ ID NO: 68):
MAGMDPGGGGAGAGSSRYFHHLLRPQQPSPLSPLSPTSHVKMEHSKMSPDKSPVGEGD
HAGGSGSGGVGGDHQPSSSAMVPVEGGSGSAGGSGSGGPTRRPRGRPPGSKNKPKPPII
VTRDSPNALHSHVLEVAGGADVVDCVAEYARRRGRGVCVLSGGGAVVNVALRQPGA
SPPGSMVATLRGRFEILSLTGTVLPPPAPPGASGLTVFLSGGQGQVIGGSVVGPLVAAGP
VVLMAASFANAVYERLPLEGEEEEVAAPAAGGEAQDQVAQSAGPPGQQPAASQSSGV
TGGDGTGGAGGMSLYNLAGNVGGYQLPGDNFGGWSGAGAGGVRPPF

### Example 14

### T3 Generation Camelina Seedlings Over-Expressing Atsob3-6 Emerged from this Deep Planting Whereas no Wild Type Plants Did

FIG. 10 shows T3 generation Camelina seedlings over-expressing Atsob3-6 (right) compared to wild type syblings (left) after being planted on 1 cm of moist Palouse silt-loam and then covered with 8 cm of dry Palouse silt loam. Ten seedlings were placed in each pot. 30 to 50% of the transgenic seedlings emerged from this deep planting whereas no wild type plants did. After this experiment was completed, it was determined that both pots experienced 100% germination. Experiment has been repeated three times.

### Example 15

### The Weight of 100 T4 Generation Camelina Seeds Over-Expressing Atsob3-6 (Right) was Determined to be Heavier Compared to Controls

FIG. 11 shows that the weight of 100 T4 generation Camelina seeds over-expressing Atsob3-6 (right) is heavier when compared to a transgenic line expressing the empty-vector (left). The transformant line (right) also yields seedlings with longer hypocotyls than empty-vector control line.

### Example 16

### The Weight of 100 Homozygous Arabidopsis sob3-6 Mutant Seeds (Left) was Determined to be Heavier when Compared to Wild-Type Control

FIG. 12 shows that the weight of 100 homozygous Arabidopsis sob3-6 mutant seeds (left) is heavier when compared to a wild-type control (right). Raw values are presented above the bars along with ±SEM.

### Example 17

### The Weight of 100 T3 Generation Transgenic Arabidopsis Seeds Over-Expressing Atsob3-6 was Determined to be Heavier when Compared to the Wild Type, and Confers a Longer Hypocotyl than the Wild-Type

FIG. 13 shows the weight of 100 T3 generation transgenic Arabidopsis seeds over-expressing Atsob3-6 compared to the wild type. Transformant-2 (far-right) is heavier when compared to the wild type (far-left) and Transformant-1 (center). Transformant-1 confers a hypocotyl phenotype that is the same as the wild type. Transformant-2 confers a longer hypocotyl than the wild-type. Raw values are presented above the bars along with ±SEM.

### Example 18

### The esc-11 Mutation also Conferred a Long Hypocotyl Phenotype in Arabidopsis T1 Transgenic Seedlings

FIG. 14 shows that the esc-11 mutation also confers a long hypocotyl phenotype in Arabidopsis T1 transgenic seedlings. The esc-11 allele was created with the same mutation as sob3-6 using site-directed-mutagenesis. Wild-type (Col-0) were transformed with an empty vector control (far-left), the wild-type copy of ESC (ESCox1 and ESCox2) or with the esc-11 allele (esc-11ox1 to esc-11ox7). The ESCox and esc-11ox alleles were driven by the CaMV35S promoter. Scale bar=5 mm.

### Example 19

### Overexpression of the SOB3 PPC Domain and the Linker Region between the PPC Domain and the AT-Hook was Sufficient to Confer a Long Hypocotyl Phenotype in T1 Transgenic Arabidopsis Seedlings

FIG. 15 shows that the overexpression of the SOB3 PPC domain and the linker region between the PPC domain and the AT-hook is sufficient to confer a long hypocotyl phenotype in T1 transgenic Arabidopsis seedlings. A wild-type (Col-0) seedling transformed with an empty vector control is shown on the left. A wild-type T1 seedling transformed with the linker region and the PPC domain driven by the CaMV35S promoter is shown on the right. Scale bar=2 mm.

1. An isolated nucleic acid encoding a polypeptide comprising SEQ ID NO:3, SEQ ID NO:6, a polypeptide having at least 93% or at least 95% sequence identity with SEQ ID NO:3, or a polypeptide having at least 75% or at least 80% sequence identity with SEQ ID NO:6.

2. The isolated nucleic acid of claim 1, wherein the nucleic acid comprises SEQ ID NO:1, SEQ ID NO:2, SEQ ID NO:4 or SEQ ID NO:5.

3. An isolated polypeptide comprising SEQ ID NO:3, SEQ ID NO:6, a polypeptide having at least 93% or at least 95% sequence identity with SEQ ID NO:3, or a polypeptide having at least 75% or at least 80% sequence identity with SEQ ID NO:6.

4. A Camelina AHL polypeptide having a mutation of the AT hook domain that confers a dominant negative phenotype as disclosed herein.

5. The Camelina AHL polypeptide of claim 4, wherein the polypeptide comprises a mutation in the AT hook domain of SEQ ID NO:3, SEQ ID NO:6, of a polypeptide having at least 93% or at least 95% sequence identity with SEQ ID NO:3, or of a polypeptide having at least 75% or at least 80% sequence identity with SEQ ID NO:6.

6. The Camelina AHL polypeptide of claim 4, wherein the polypeptide lacks the AT hook domain thereof.

7. The Camelina AHL polypeptide of claim 6, wherein the polypeptide comprises an intact or functional PPC domain, and preferably additionally comprising the linker region between the PPC domain and the AT-hook domain.

8. A method of generating modified plants, seedlings or seeds, comprising introducing into, or engineering in a plant cell, a nucleic acid encoding a mutant AHL protein having a mutation of the AT hook domain that confers a dominant negative phenotype as disclosed herein, provided that if the mutant AHL protein comprises an Arabadodpis thaliana (AT) Sob3 mutant, that the plant cell is not an AT plant cell.

9. The method of claim 8, wherein the mutant AHL protein comprises a mutant Sob3 or Esc polypeptide, or an ortholog, paralog or homolog thereof.

10. The method of claim 8, wherein introducing into, or engineering in comprises at least one of plant breeding and recombinant DNA and/or transformation methods.

11. The method of claim 8, wherein the mutant AHL protein is based on, or derived from a Camelina, or Arabadodpis thaliana (AT) AHL protein, or from a Oryza sativa (Rice); Sorghum bicolor (sorghum); and Zea mays (maize), Brassica rapa, or Vitis vinifera AHL protein.

12. The method of claim 8, wherein the plant cell is that of Brassica, Arabidopsis, soybean (Glycine max), canola (Brassica napus or B. rapa), sunflower (Helianthus annuus), Crambe (Crambe abysinnica); Black Mustard; Yellow Mustard (Sinapis alba); Oriental Mustard (Brassica juncea); Broccoli (Brassica oleracea italica); Rapeseed (Brassica napus); Meadowfoam (Limnanthes alba), Radish (Raphanus sativus); Wasabi (Wasabia japonica); Horseradish (Cochlearia Armoracia); Cauliflower; Garden cress (Lepidium sativum); Watercress (Nasturtium officinalis); and Papaya (Carica papaya), canola (rape), wheat (triticum), rice, corn, or a monocot.

13. The method of claim 8, wherein the phenotype comprises at least one of taller seedlings, and heavier seeds.

14. A recombinant or genetically modified plant or plant cell comprising a nucleic acid encoding a mutant AHL polypeptide having a mutation of the AT hook domain that confers a dominant negative phenotype as disclosed herein, provided that if the mutant AHL protein is an Arabadodpis thaliana (AT) Sob3 mutant, the plant or plant cell is not an AT plant or plant cell.

15. The recombinant or genetically modified plant or plant cell of claim 14, wherein the mutant AHL protein comprises a mutant Sob3 or Esc polypeptide, or an ortholog, paralog or homolog thereof.

16. The recombinant or genetically modified plant or plant cell of claim 14, wherein the mutant AHL protein is based on, or derived from a Camelina, or Arabadodpis thaliana (AT) AHL protein or from a Oryza sativa (Rice); Sorghum bicolor (sorghum); and Zea mays (maize), Brassica rapa, or Vitis vinifera AHL protein.

17. The recombinant or genetically modified plant or plant cell of claim 14, wherein the phenotype of the plant comprises at least one of taller seedlings, and heavier seeds.

18. The recombinant or genetically modified plant or plant cell of claim 14, wherein the plant is derived using a method according to any one of claims 8-13.
